(19) United States
(12) Patent Application Publication
(10) Pub. No.: US 2026/0055394 A1
(43) Pub. Date: Feb. 26, 2026

(54) BARCODED INFLUENZA VIRUSES AND MUTATIONAL SCANNING LIBRARIES INCLUDING THE SAME

(71) Applicant: Fred Hutchinson Cancer Center, Seattle, WA (US)

(72) Inventors: Andrea Loes, Seattle, WA (US); Frances Welsh, Seattle, WA (US); Jesse Bloom, Seattle, WA (US)

(73) Assignee: Fred Hutchinson Cancer Center, Seattle, WA (US)

(21) Appl. No.: 19/103,482

(22) PCT Filed: Aug. 11, 2023
(86) PCT No.: PCT/US2023/072122

§ 371 Date: Feb. 12, 2025

Related U.S. Application Data
(60) Provisional application No. 63/371,369, filed on Aug. 12, 2022.

(51) Int. Cl. C12N 15/10

(52) U.S. Cl. CPC C12N 15/1065

(57) ABSTRACT

Methods to create barcoded influenza viruses without disrupting the function of the viral proteins and the proper packaging of the viral genome segments are described. The barcoded influenza viruses can be used within deep mutational scanning libraries to map influenza resistance mutations to therapeutic treatments. The libraries can also be used to predict influenza strains that may become resistant to therapeutic treatments and/or more easily evolve to infect new species.

### CROSS REFERENCE TO RELATED APPLICATION

This application is a U.S. National Phase Patent Application based on International Patent Application No. PCT/US2023/072122, filed on Aug. 11, 2023, which claims the benefit of U.S. Provisional Patent Application No. 63/371,369, filed Aug. 12, 2022, the entire contents each of which are incorporated by reference herein in their entirety.

### STATEMENT REGARDING FEDERALLY SPONSORED RESEARCH OR DEVELOPMENT

This invention was made with government support under grant numbers Al165821 and 75N93021C00015, awarded by the National Institutes of Health. The government has certain rights in the invention.

### STATEMENT REGARDING SEQUENCE LISTING

The Sequence Listing associated with this application is provided in XML format in lieu of a paper copy and is hereby incorporated by reference into the specification. The name of the XML file containing the Sequence Listing is 3DP7600.XML. The XML file is 102,924 bytes, was created on Feb. 12, 2025, and is being submitted electronically via Patent Center.

### FIELD OF THE DISCLOSURE

Described are barcoded viruses and methods of producing the same. Specifically, barcoded influenza viruses with barcodes that do not disrupt the function of the viral proteins and the proper packaging of the viral genome segments are described.

### BACKGROUND OF THE DISCLOSURE

While vaccination has all but eliminated smallpox and polio, the on-going mutation of other viruses continues to pose significant health threats. For example, there are approximately sixty known influenza viruses and the predominance of any particular strain changes every year, requiring influenza vaccines to be continually updated to be effective. Other viruses such as human immunodeficiency virus (HIV), Ebola virus, and Middle Eastern respiratory syndrome coronavirus (MERS-COV) also continue to pose significant health threats. To combat the spread of viruses, tools are needed to evaluate when drugs, vaccines, or antibodies are effectively working against viral proteins, or conversely, when viral proteins have or are likely to develop resistance to these countermeasures and pose a greater risk.

Mutations in viral proteins allow viruses to continue to evolve and potentially increase virulence and develop resistance to treatments or vaccines. Proteins are made of strings of amino acids with different proteins having different numbers and orders of amino acids. Altering amino acids at different positions through mutagenesis can help identify those amino acids that are essential to the function of the protein and provide understanding of the impact of mutations on drug resistance, immune escape, vaccination efficacy, and pathogenesis. Another tool in assessing viral function is mutational scanning including deep mutational scanning which uses high-throughput screening to assess the function of a large number of protein variants.

Viruses may have segmented and non-segmented genomes. Segmentation of viral genomes allows the exchange of intact genes between related viruses when they coinfect the same cell. In viruses with segmented genomes like, for example, the influenza virus, replication occurs in the nucleus and the RNA-dependent RNA polymerase (RdRp) produces one monocistronic messenger RNA (mRNA) strand (encoding one polypeptide per RNA molecule) from each genome segment. Each genome segment includes a promoter sequence, segment-specific non-coding regions adjacent to the promoter region, and open reading frame coding sequences that encode particular viral proteins. Each segment also includes a packaging signal on each end of the viral RNA (vRNA) (referred to as the 5′ end and the 3′ end) that is specific to each genomic segment.

In natural virion form, all viruses contain nucleic acid (DNA or RNA) encased in a protein coat called a capsid. Once a virus enters a host's body, the first step in infecting cells is binding of the virion's viral entry protein to a host cell. This binding is followed by fusion of the virion with the host cell and transfer of the viral DNA or RNA into the host cells. Once the viral DNA or RNA enters the host's cells, viruses begin to multiply using the host's ribosomes to generate viral proteins. For many human pathogenic viruses, the binding and fusion steps are performed by a single viral entry protein. For example, the influenza virus uses a single-entry protein for binding and fusion with a host cell, hemagglutinin.

Viral entry proteins are a primary target of immune system responses against viral infections. Most vaccines elicit neutralizing antibodies to the viral entry protein. Therapeutic antibodies can also be used to impair the activity of viral entry proteins, with the potential to both protect against infection as well as to therapeutically treat active infection. However, viral entry proteins mutate and evolve over time, and mutations can allow these proteins to escape recognition by immune system responses and therapeutic antibodies.

A virus' viral entry protein is also a key determinant of the species that the particular virus can infect, and adaptive evolution of these entry proteins has been retrospectively characterized in most molecularly documented examples of non-human viruses jumping into humans. For example, the influenza pandemics of 1918, 1957, and 1968 all involved mutations that turned viral entry proteins from avian viral strains to strains that could better infect humans.

After entry, new viral capsids are assembled with capsomere proteins, a subunit of the capsid. The negative sense RNA strands combine with capsids and viral RdRp to form new negative sense RNA virions. After assembly and maturation of the nucleocapsid, the new virions exit the cell in a variety of ways. They may exit through budding in which part of the host cell membrane becomes part of the virus and breaks off from the cell, exocytosis in which substances are secreted through the host cell membrane, or lysis, in which the cell membrane is ruptured. Once the viruses have exited the cell, they continue to spread.

As viral entry proteins are a primary target of immune system responses, mapping functional and antigenic effects of mutations of the entry proteins plays a role in the design of therapeutic agents and vaccines. While deep mutational scanning has been used to completely map functional and antigenic effects of all mutations to the entry proteins of influenza virus and HIV, the sequencing methods that are currently used (e.g., Illumina sequencing) can have an error rate that is too high to produce informative and reliable results without complex and expensive error-correction strategies. Alternative methods (such as PacBio) lack the throughput and/or accuracy to efficiently (and affordably) characterize diverse libraries at multiple conditions. One solution is to associate each variant in a library with a unique nucleotide barcode (Hiatt, et al. Nat Methods 7:119-122 (2010)). The barcodes can then be sequenced using standard sequencing (e.g., Illumina) to read out the library composition.

US2021/0147832 describes methods to barcode influenza viruses without affecting viral fitness. Before the disclosure of US2021/0147832, barcoding viruses without disrupting viral fitness was thought to be difficult due to the highly constrained genome packaging mechanism of influenza viruses (Hutchinson et al., J. Gen. Virol. 91 (2) (2010), doi: 10.1099/vir.0.017608-0). US2021/0147832 particularly described (i) duplicating and inserting a copy of the 5′ vRNA packaging signal between the end of the corresponding viral genome segment's open reading frame (corresponding to the stop codon of the transcribed positive sense mRNA) and the naturally occurring non-coding portion of the 5′ vRNA packaging signal; and (ii) inserting the nucleic acid barcode between the end of the viral genome segment's open reading frame (corresponding to the stop codon of the transcribed positive sense mRNA) and the inserted copy of the 5′ vRNA packaging signal. This approach is efficient and cheap and provides a linkage between barcode and variant. Unfortunately, however, barcodes can occasionally be deleted from the influenza segment during viral replication, resulting in non-barcoded virion growth introducing experimental background noise into results making interpretation more difficult.

### SUMMARY OF THE DISCLOSURE

The current disclosure provides methods to reduce the deletion of the barcode region and also to reduce virion replication following barcode loss. These methods increase the experimental power of barcoded influenza viral libraries, allowing higher throughput and more efficient and accurate interpretation of results.

Particular embodiments include two key aspects: (i) duplicating and inserting a copy of the 5′ vRNA packaging signal between the end of the corresponding viral genome segment's open reading frame (corresponding to the stop codon of the transcribed positive sense mRNA) and the naturally occurring non-coding portion of the 5′ vRNA packaging signal, wherein the coding region of the packaging signal that is within the genomic segment's open reading frame is recoded to have less than 70% sequence identity with duplicated region of the 5′ vRNA packaging signal; and (ii) inserting the nucleic acid barcode between the end of the viral genome segment's open reading frame (corresponding to the stop codon of the transcribed positive sense mRNA) and the inserted copy of the 5′ vRNA packaging signal. The copy of the sequence that is within the protein coding region of the genomic segment such as the influenza genomic segment, is recoded to reduce sequence similarity with the terminal packaging signal. Guanine cytosine content is monitored to ensure that the final recoded region has a similar guanine cytosine content to the genomic segment such as the influenza genomic segment. Recoding the coding region of the packaging signal that is within the genomic segment's open reading frame to have less than 70% sequence identity with duplicated region of the 5′ vRNA packaging signal reduces the loss of barcodes during viral replication.

The methods disclosed herein provide another advance by inserting at least one stop codon into the vRNA so that if a barcode is not present, a functional virion will not form. In certain examples, the methods include inserting at least one stop codon in the copy of a 5′ viral RNA genome packaging signal that is present after the inserted barcode sequence.

The incorporation of the nucleotide barcode according to the methods disclosed herein allows for the generation of libraries of influenza virions each carrying a unique barcode linked to a different viral protein sequence. By sequencing the barcode, it is possible to identify the full sequence of the viral gene. These libraries can then be used with large-scale sequencing technology to make parallel measurements of how mutations to the viral proteins affect viral growth and immune recognition. For example, the barcoded influenza viruses can be used within deep mutational scanning libraries to map influenza resistance mutations to therapeutic treatments and can be used to make parallel measurements against a defined set of recently circulating or historically relevant influenza strains. The libraries can also be used to predict influenza strains that may become resistant to therapeutic treatments and/or more easily evolve to infect new species. The libraries include features that allow efficient collection and assessment of informative data. While the methods described herein may be used for a variety of viruses, exemplary embodiments are shown using influenza.

The current disclosure also provides barcoded genomic segments for distantly related, functional influenza hemagglutinins (HAs) from low pathogenicity avian influenza viruses that produce virions that grow well in tissue-culture can be used as internal standards for experiments with barcoded viruses such as influenza. Distance is a property that measures evolutionary-relatedness and sequence similarity to strains recently circulating in humans, e.g. H1 and H3. The closer this distance, the greater the probability of cross-reactive antibodies in humans that will be able to bind to both antigens of both strains. Most humans have limited neutralization activity against distant HAs. When performing selection experiments that manipulate the natural selection process, the relative growth of library variants in the presence and absence of selective pressure may be analyzed. Including a distant HA as an internal standard for non-neutralized virus growth allows for quantitative measurement of the impact of mutations on neutralization. A nucleic acid standard may also be used. In particular examples, a nucleic acid standard may be generated with in vitro transcription to resemble the vRNA of influenza. In addition, the relative frequencies of mutants or variants with respect to these controls at various concentrations may be used to calculate a measurement akin to an IC50 (half maximal inhibitory concentration) from a neutralization assay (which is currently the standard approach for assessing inhibition of infection by serum or antibodies) can be used. This system was developed to use large-scale sequencing technology (next generation sequencing (NGS)) and allows for massively parallel neutralization assays with the barcoded influenza variant libraries. The ability to use NGS as a readout for neutralization assays allows for the evaluation of neutralization potency against some or all variants included in a library simultaneously, within a single dilution series. Analysis of sequence data from these experiments allows for the generation of IC50-like measurements for multiple viruses at once, using the same volume of sample that is currently used to generate an IC50 against a single virus. This advancement allows generation of significantly more measurements so that more detailed information about immune specificity of a given sample against many viruses can be obtained. Such screening methods allow for more measurements even in samples with limited volume. Taken together, the disclosed barcoded viruses and resulting mutational scanning libraries provide an important advance in the ability to generate, store, and characterize a large number of variant viral proteins.

To the accomplishment of the foregoing and related ends, certain illustrative aspects of the methods and compositions are described herein in connection with the following description and the attached drawings. The summary is provided to introduce a selection of concepts in a simplified form that are further described below in the Detailed Description.

### BRIEF DESCRIPTION OF THE SEVERAL VIEWS OF THE DRAWINGS

FIGS. 1A, 1B. Barcoded influenza virus vRNA with packaging signals decoupled from the coding sequence. In FIG. 1A, a sufficient sequence of the 5′ end of the viral RNA (which is the 3′ end of the mRNA transcribed from the negative sense vRNA depicted in the FIG.) is duplicated (typically >90 nucleotides). In FIG. 1B, a sufficient sequence of the 3′ end of the viral RNA is duplicated (typically >90 nucleotides) and inserted at the 3′ end of the viral segment.

FIG. 2. Depiction of a plasmid barcoded according to methods of the current disclosure.

FIG. 3. Data demonstrating that the barcoding strategies described herein are selectively neutral and have minimal effects on viral fitness.

FIGS. 4A-4C. Depiction of measuring antibody neutralization curves using deep sequencing of viral libraries and visualizing the results. (FIG. 4A) Viral variants are either treated with an antibody or left untreated. At each antibody concentration, a specific fraction of each viral variant survives neutralization. Here all but the V1K variant are mostly neutralized. (FIG. 4B) By measuring the fraction surviving at several concentrations, a neutralization curve can be interpolated. The middle vertical dashed line is the concentration corresponding to the scenario in FIG. 4A. (FIG. 4C) When curves for many mutants have been measured, it is more informative to show the resulting measurements in logo plots (Adapted from Doud et al. (2018) bioRxiv DOI: 210468). The height of each letter is the fraction of variants with that mutation that survive at the antibody concentrations indicated by vertical lines in FIG. 4B.

FIG. 5. The functional effects of all mutations can be mapped in cells from relevant host species. For instance, a natural animal reservoir can be bats and the relevant test species can be humans. Species-specific maps of mutational effects can be used to inform sequence-based methods to identify viral host adaptation. For example, in the logo plots (I), at the 4th site, amino acid E is favored in bat cells but amino acid K is favored in human cells. New influenza viral sequences may be scored for their adaptation to each host (II).

FIGS. 6A, 6B. Scoring host adaptation. (FIG. 6A) Viruses are adapted to their long-standing animal reservoirs. When they jump to humans, they initially may be poorly adapted. (FIG. 6B) Host adaptation may be scored based on sequence, and adaptation after a jump may be charted.

FIG. 7. Schematic of barcoded construct design showing incorporation of barcodes into influenza genomic segments, such as hemagglutinin (HA).

FIG. 8. Neutralization curve showing that there is limited neutralization of an example distant HA (tissue culture (TC)-adapted, chimeric H6/A/Turkey/Massachusetts/1965) by pooled human serum. Fraction of reads corresponding to non-neutralized HA virus neutralization standard or RNA spike-in standard control.

FIGS. 9A, 9B. (FIG. 9A) non-neutralized HA virus neutralization standard, or (FIG. 9B) RNA spike-in standard control correlated with concentration of pooled serum used for selection. Normalizing to the neutralization standard at each concentration for each sequencing sample, an IC50-like measurement may be calculated for each barcoded variant included in the library.

FIGS. 10A, 10B. Graphs depicting (FIG. 10A) neutralization by an example monoclonal antibody is impacted by mutations in the library as shown with fluorescence-based neutralization assays and (FIG. 10B) similar measurements for these strains are obtained using the NGS-based neutralization assay method.

FIG. 11. Exemplary sequences supporting the disclosure: Packaging Signal at 5′ end for Influenza A virus Segment 4 (SEQ ID NO: 1); Packaging Signal at 5′ end for Influenza A virus Segment 6 (SEQ ID NO: 3); Influenza A virus (A/Puerto Rico/8/1934(H1N1)) segment 4 (NCBI Ref Seq: NC_002017.1; (SEQ ID NO:5). The coding sequence for the gene HA is in bold. Influenza A virus (A/Puerto Rico/8/1934(H1N1)) segment 6 (NCBI Ref Seq: NC_002018.1; SEQ ID NO: 6). The coding sequence for the gene NA is in bold. Influenza A virus (A/New York/392/2004(H3N2)) segment (NCBI Ref Seq: NC_007366.1; SEQ ID NO: 7). The coding sequence for the gene HA is in bold. Influenza A virus (A/New York/392/2004(H3N2)) segment 6 (NCBI Ref Seq: NC_007368.1; SEQ ID NO: 8). The coding sequence for the gene NA is in bold. Influenza A virus (A/goose/Guangdong/1/1996(H5N1)) hemagglutinin (HA) gene (NCBI Ref Seq: NC_007362.1; SEQ ID NO: 9). The coding sequence for the gene HA is in bold. Influenza A virus (A/Goose/Guangdong/1/96(H5N1)) neuraminidase (NA) gene (NCBI Ref Seq: NC_007361.1; SEQ ID NO: 10). The coding sequence for the gene NA is in bold. Influenza B virus (B/Lee/1940) segment 4 (NCBI Ref Seq: NC_002207.1; SEQ ID NO:11). The coding sequence for the gene HA is in bold. Influenza B virus (B/Lee/1940) segment 6 (NCBI Ref Seq: NC_002209.1; SEQ ID NO: 12). The coding sequence for the gene NB is in bold; the coding sequence for the gene NA is underlined. Influenza A virus (A/Puerto Rico/8/1934(H1N1)) segment 1 (NCBI Ref Seq: NC_002023.1; SEQ ID NO: 13). The coding sequence for the gene PB2 is in bold. Influenza A virus (A/Puerto Rico/8/1934(H1N1)) segment 2 (NCBI Ref Seq: NC_002021.1; SEQ ID NO: 14). The coding sequence for the gene PB1 is in bold; the coding sequence for the gene PB1-F2 is underlined. Influenza A virus (A/Puerto Rico/8/1934(H1N1)) segment 3 (NCBI Ref Seq: NC_002022.1; SEQ ID NO: 15). The coding sequence for the gene PA is in bold. Influenza A virus (A/Puerto Rico/8/1934(H1N1)) segment 5 (NCBI Ref Seq: NC_002019.1; SEQ ID NO: 16). The coding sequence for the gene NP is in bold. Influenza A virus (A/Puerto Rico/8/1934(H1N1)) segment 7 (NCBI Ref Seq: NC_002016.1; SEQ ID NO: 17). The coding sequence for the gene M2 is in bold; the coding sequence for the gene M1 is underlined. Influenza A virus (A/Puerto Rico/8/1934(H1N1)) segment 8 (NCBI Ref Seq: NC_002020.1; SEQ ID NO: 18). The coding sequence for the gene NS2 is in bold; the coding sequence for the gene NS1 is underlined. Influenza A virus (A/New York/392/2004(H3N2)) segment 1 (NCBI Ref Seq: NC_007373.1; SEQ ID NO: 19). The coding sequence for the gene PB2 is in bold. Influenza A virus (A/New York/392/2004(H3N2)) segment 2 (NCBI Ref Seq: NC_007372.1; SEQ ID NO: 20). The coding sequence for the gene PB1 is in bold; the coding sequence for the gene PB1-F2 is underlined. Influenza A virus (A/New York/392/2004(H3N2)) segment 3 (NCBI Ref Seq: NC_007371.1; SEQ ID NO: 21). The coding sequence for the gene PA is in bold; the coding sequence for the gene PA-X is underlined. Influenza A virus (A/New York/392/2004(H3N2)) segment 5 (NCBI Ref Seq: NC_007369.1; SEQ ID NO: 22). The coding sequence for the gene NP is in bold. Influenza A virus (A/New York/392/2004(H3N2)) segment 7 (NCBI Ref Seq: NC_007367.1; SEQ ID NO: 23). The coding sequence for the gene M2 is in bold; the coding sequence for the gene M1 is underlined. Influenza A virus (A/New York/392/2004(H3N2)) segment 8 (NCBI Ref Seq: NC_007370.1; SEQ ID NO: 24). The coding sequence for the gene NS2 is in bold; the coding sequence for the gene NS1 is underlined. Influenza A virus (A/Goose/Guangdong/1/96(H5N1)) polymerase (PB2) gene (NCBI Ref Seq: NC_007357.1; SEQ ID NO: 25). The coding sequence for the gene PB2 is in bold. Influenza A virus (A/goose/Guangdong/1/1996(H5N1)) polymerase (PB1) and PB1-F2 protein (PB1-F2) genes (NCBI Ref Seq: NC_007358.1; SEQ ID NO: 26). The coding sequence for the gene PB1 is in bold; the coding sequence for the gene PB1-F2 is underlined. Influenza A virus (A/goose/Guangdong/1/1996(H5N1)) polymerase (PA) and PA-X protein (PA-X) genes (NCBI Ref Seq: NC_007359.1; SEQ ID NO: 27). The coding sequence for the gene PA is in bold; the coding sequence for the gene PA-X is underlined. Influenza A virus (A/Goose/Guangdong/1/96(H5N1)) nucleocapsid protein (NP) gene (NCBI Ref Seq: NC_007360.1; SEQ ID NO: 28). The coding sequence for the gene NP is in bold. Influenza A virus (A/goose/Guangdong/1/1996(H5N1)) segment 7 (NCBI Ref Seq: NC_007363.1; SEQ ID NO: 29). The coding sequence for the gene M2 is in bold; the coding sequence for the gene M1 is underlined. Influenza A virus (A/goose/Guangdong/1/1996(H5N1)) segment 8 (NCBI Ref Seq: NC_007364.1; SEQ ID NO: 30). The coding sequence for the gene NS2 is in bold; the coding sequence for the gene NS1 is underlined. Influenza B virus RNA 1 (NCBI Ref Seq: NC_002204.1; SEQ ID NO:31). The coding sequence for the gene PB1 is in bold. Influenza B virus (B/Lee/1940) segment 2 (NCBI Ref Seq: NC_002205.1; SEQ ID NO:32). The entire sequence encodes PB2. Influenza B virus (B/Lee/1940) segment 3 (NCBI Ref Seq: NC_002206.1; SEQ ID NO:33). The coding sequence for the gene PA is in bold. Influenza B virus (B/Lee/1940) segment 5 (NCBI Ref Seq: NC_002208.1; SEQ ID NO:34). The coding sequence for the gene NP is in bold. Influenza B virus (B/Lee/1940) segment 7 (NCBI Ref Seq: NC_002210.1; SEQ ID NO: 35). The coding sequence for the gene M1 is in bold. Influenza B virus (B/Lee/1940) segment 8 (NCBI Ref Seq: NC_002211.1; SEQ ID NO:36). The coding sequence for the gene NS2 is in bold; the coding sequence for the gene NS1 is underlined. Sequence for internal controls and flanking regions include: Chimeric_H6/Turkey/Massachusetts/3740/1965_A151D_Protein (SEQ ID NO: 39); Chimeric_H6/Turkey/Massachusetts/3740/1965_A151D_V2_Protein (SEQ ID NO: 40); Chimeric_H6/Turkey/Massachusetts/3740/1965_R235M_Protein (SEQ ID NO: 41); H8/Mallard/Sweden/24/2002_E408G_Protein* (SEQ ID NO: 42); Constantregion_U12-signalpeptide_packagingsignal_nucleotide (SEQ ID NO: 43); Constantregion_packagingsignal-U13_v1_nucleotide (SEQ ID NO: 44); and Constantregion_packagingsignal-U13_v2_nucleotide (SEQ ID NO: 45).

### DETAILED DESCRIPTION

Viral evolution and cross-species transmission poses a major challenge for the design of long-lasting vaccines. For example, enough variants for influenza arise that the strains included in the influenza vaccine are assessed every year, and frequently updated as the virus evolves to escape the pre-existing immunity elicited by prior infections or vaccinations (Bedford et al., Nature 523 (7559), 217-20 (2015)). Understanding how mutations affect a virus's inherent fitness and its antigenicity is therefore important for forecasting viral evolution for vaccine-strain selection (Łuksza & Lässig, Nature 507, 57-61 (2014)) and guiding the development of vaccines (Krammer, Nat. Rev. Immunol. 19, 383-397 (2019)) and antivirals (Koszalka et al., Influenza Other Respi. Viruses 11 (3), 240-46 (2017)).

Mutational scanning is a powerful approach for measuring the effects of large numbers of mutations (Fowler & Fields, Nat. Methods 11 (8), 801-7 (2014)). As an example, deep mutational scanning has been applied to measure how mutations to influenza virus affect viral growth in cell culture (Doud & Bloom, Viruses, 8 (6), 1-17 (2016); Wu et al., Sci. Rep. 4, Article No. 4942 (2014); Lee et al., Proc. Natl. Acad. Sci. USA (2018), doi: 10.1073/pnas. 1806133115), viral neutralization by antibodies (Doud et al., PLOS Pathog. 13 (2017), doi: 10.1371/journal.ppat. 1006271), and viral neutralization by polyclonal human sera (Lee et al., Mapping person-to-person variation in viral mutations that escape polyclonal serum targeting influenza hemagglutinin, 1-28 (2019)). This work can advance the aforementioned goals of improving forecasting of viral evolution and guiding the development of vaccines and antivirals.

Recently, deep mutational scanning of non-viral genes has been greatly improved by new approaches that involve linking a short random-nucleotide barcode to the full gene variant (Hiatt et al., Nature Methods, 7 (2), 119-122 (2010); Starita et al., Genetics, 200 (2), 413-422 (2015); Kitzman, et al., Nature Methods, 12 (3), 203-206 (2015)). Barcoding viral genomic segments reduces costs and labor of deep mutational scanning by increasing the throughput of diagnostic samples, shortening processing time, diminishing the risk of technical batch effects, lowering library preparation costs and per-sample cost, and increasing the accuracy of results. Barcodes could be linked to individual variants with long-read sequencing in DNA plasmid samples, and Illumina sequencing of barcodes alone in downstream selection steps would allow for the measurement of the effects of mutations on viral fitness. Similar approaches in non-viral systems have been used (Kitzman et al., Nat. Methods 12 (3) 203-6 (2015); Starita, et al., American Journal of Human Genetics 103, 498-508 (2018)).

US2021/0147832 describes methods to barcode viruses without affecting viral fitness. Before the disclosure of US2021/0147832, barcoding influenza viruses without disrupting viral fitness was thought to be difficult due to the highly constrained genome packaging mechanism of viruses (Hutchinson et al., J. Gen. Virol. 91 (2) (2010), doi: 10.1099/vir.0.017608-0). US2021/0147832 particularly described (i) duplicating and inserting a copy of the coding region of the 5′ vRNA packaging signal between the end of the corresponding viral genome segment's open reading frame (corresponding to the stop codon of the transcribed positive sense mRNA) and the naturally occurring non-coding portion of the 5′ vRNA packaging signal; and (ii) inserting the nucleic acid barcode between the end of the viral genome segment's open reading frame (corresponding to the stop codon of the transcribed positive sense mRNA) and the inserted copy of the coding region 5′ vRNA packaging signal. This approach is efficient and cheap and provides a linkage between barcode and variant. Unfortunately, however, barcodes can be deleted during viral replication, resulting in non-barcoded virion growth introducing experimental background noise into results making interpretation more difficult.

The current disclosure provides methods to reduce the loss of barcodes during viral replication and also to reduce virion growth and survival when a barcode is lost. These methods increase the experimental power of barcoded viral libraries, allowing higher throughput and more efficient and accurate interpretation of results.

Particular embodiments include two key aspects: (i) duplicating and inserting a copy of the coding region 5′ vRNA packaging signal between the end of the corresponding viral genome segment's open reading frame (corresponding to the stop codon of the transcribed positive sense mRNA) and the naturally occurring non-coding portion of the 5′ vRNA packaging signal, wherein the copy of the packaging signal that is within the open reading frame of the viral genomic segment is recoded to have less than 70% sequence identity with the terminal 5′ vRNA packaging signal; and (ii) inserting the nucleic acid barcode between the end of the viral genome segment's open reading frame (corresponding to the stop codon of the transcribed positive sense mRNA) and the inserted copy of the coding region of the 5′ vRNA packaging signal, wherein the copy of the coding region of 5′ vRNA packaging signal which is within the open reading frame of the genomic segment is recoded to have less than 70% sequence identity with the region of 5′ vRNA packaging signal that is duplication after the barcode to reduce the loss of barcodes during virus replication. Further, in particular embodiments, the copy of the sequence that is within the protein coding region is evaluated to consider human codon usage to limit the impact on protein expression, In addition, guanine cytosine content is monitored to ensure that the final recoded region has a similar guanine cytosine content to the genomic segment of the virus of interest such as the influenza genomic segment. In additional embodiments, one or more additional the stop codon for the open reading frame, within the region of the copy of a 5′ viral RNA genome packaging signal that contains sequence which encodes for the c-terminal region of the protein sequence.

In particular embodiments, the copy of the 5′ vRNA packaging signal has less than 70% sequence identity, less than 65% sequence identity, less than 60% sequence identity, less than 55% sequence identity, less than 50% sequence identity, less than 45% sequence identity, less than 40% sequence identity, less than 35% sequence identity, less than 30% sequence identity, or less than 25% sequence identity with the 5′ vRNA packaging signal.

In particular embodiments, the copy of the 5′ vRNA packaging signal has 65%-75% sequence identity, 60%-65% sequence identity, 40% to 75% sequence identity, 45% to 65% sequence identity, 55%-60% sequence identity, 50%-55% sequence identity, 45%-50% sequence identity, 40%-45% sequence identity, 35%-40% sequence identity, 30%-35% sequence identity, 25%-30% sequence identity, or 20%-25% sequence identity with the 5′ vRNA packaging signal.

In particular embodiments, the copy of the 5′ vRNA packaging signal has 70% sequence identity, 69% sequence identity, 68% sequence identity, 67% sequence identity, 66% sequence identity, 65% sequence identity, 64% sequence identity, 63% sequence identity, 62% sequence identity, 61% sequence identity, 60% sequence identity, 59% sequence identity, 580% sequence identity, 57% sequence identity, 56% sequence identity, 55% sequence identity, 54% sequence identity, 53% sequence identity, 52% sequence identity, 51% sequence identity, 50% sequence identity, 49% sequence identity, 48% sequence identity, 47% sequence identity, 46% sequence identity, 45% sequence identity, 44% sequence identity, 43% sequence identity, 42% sequence identity, 41% sequence identity, 40% sequence identity, 39% sequence identity, 38% sequence identity, 37% sequence identity, 36% sequence identity, or 35% sequence identity, with the 5′ vRNA packaging signal.

In particular embodiments, the length of the copy of the coding region of the 5′ vRNA packaging signal is the same length as the native 5′ vRNA packaging signal. In other aspects, it may be shorter or longer than the length of the native 5′ vRNA packaging signal. For example the copy of the 5′ vRNA packaging signal may be 90% of the length of the native 5′ vRNA packaging signal, 80% of the length of the native 5′ vRNA packaging signal, 75% of the length of the native 5′ vRNA packaging signal; 60% of the length of the native 5′ vRNA packaging signal, 50% of the length of the native 5′ vRNA packaging signal.

In particular embodiments, the copy of the 5′ vRNA packaging signal has 70-200 nucleotides, 75-180 nucleotides, 75-150 nucleotides, 80-100 nucleotides, greater than 85, 86, 87, 88, 89, 90, 91, 92, 93, 94, 95, 96, 97, 98, 99, 100, 102, 103, 104, 105, 106, 107 nucleotides, specifically 85, 86, 87, 88, 89, 90, 91, 92, 93, 94, 95, 96, 97, 98, 99, 100, 102, 103, 104, 105, 106, 107, 108, 109, 110, 112, 113, 114, 115, 116, 117, 118, 119, 120 nucleotides.

The methods disclosed herein provide another advance by inserting at least one stop codon into the vRNA so that a functional virion will not form even if the barcode is removed. In certain examples, the methods include inserting at least one stop codon in the duplicated coding region of a 5′ viral RNA genome packaging signal after the stop codon for the open reading frame and the incorporated barcode.

In certain examples, the methods include inserting 1, 2, 3, 4, or 5 contiguous or noncontiguous stop codons in the coding region of the terminal packaging signal. In certain embodiments, the methods include inserting a plurality of stop codons including 1, 2, 3, 4, or 5 stop codons after a stop codon for the open reading frame in the copy of a 5′ viral RNA genome packaging signal. In some examples, stop codons are added within the region of the packaging signal that would typically be part of the open reading frame, but is now after the open reading frame stop codon and barcode, that is, stop codons are inserted in one or more locations in the non-recoded, duplicated copy of the packaging signal. The number of stop codons in each region may be the same or different and the stop codons in each region may be contiguous or non-contiguous.

In certain examples, the methods include inserting 1 stop codon in the non-recoded, duplicated copy of the packaging signal. In certain examples, the methods include inserting 2 stop codons in the non-recoded, duplicated copy of the packaging signal. In certain examples, the methods include inserting 3 stop codons in the non-recoded, duplicated copy of the packaging signal. The stop codons inserted in the non-recoded, duplicated copy of the packaging signal may be contiguous or non-contiguous.

For example, SEQ ID NO: 44 is an example of the constant recoded region used in the H1 libraries. It includes the recoded region of the coding region of the packaging signal, the barcode, and the terminal packaging signal with the added stop codon in that order. The terminal packaging signal is the viral packaging signal such as an influenza packaging signal. The terminal region retains high sequence identity with the packaging signal of the original unmodified segment for the gene of the viral strain such as influenza that is being made with an incorporated barcode. For example, it may have 80% sequence identity, 85% sequence, 90% sequence identity, 95% sequence identity, 96% sequence identity, 97% sequence identity, 98% sequence identity, or 99%, 100% sequence identity to the protein, nucleic acid, or gene sequences disclosed herein. The duplicated region, or internal copy of a portion of the packaging signal, retains amino acid identity to the subtype of the influenza gene that is being produced, however, the recoding of the nucleotide sequence within the region is done to reduce similarity with the terminal packaging signal. While not wishing to be bound, it is theorized that this reduces the likelihood of the barcodes being deleted from the segment during viral replication. In some aspects, the packaging signal is from a different subtype than the protein sequence of the gene within the barcoded segment. In such cases, neither the amino acid sequence nor the nucleotide sequence is maintained between the copy of the packaging signal and this region of the open reading frame. An example of the 3′ region of the vRNA that is kept constant is shown in SEQ ID NO: 43.

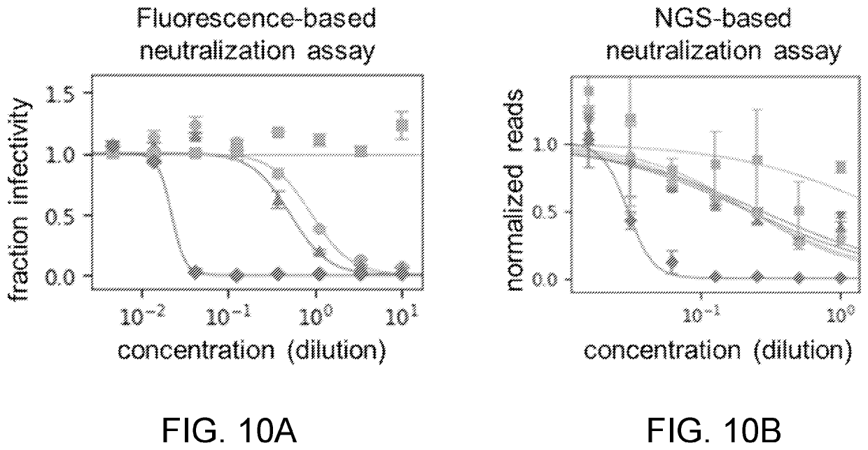

In addition, the current disclosure provides barcoded genomic segments for distantly related, TC-adapted, functional influenza hemagglutinins (HAs) that can be used in HA libraries as internal standards for experiments with barcoded viruses such as influenza. Most humans have limited neutralization activity against these distant HAs. In other aspects, distantly related neuraminidase could be used in a neuraminidase library as an internal standard for experiments with barcoded viruses such as influenza. When performing selection experiments, the relative growth of library variants in the presence and absence of a selective pressure can be analyzed. Including a distant HA as an internal standard for non-neutralized virus growth allows for quantitative measurement of the impact of mutations on neutralization. In addition, the relative frequencies of mutants or variants with respect to this control at various concentrations may be used to calculate a measurement akin to an IC50 (half maximal inhibitory concentration) from a neutralization assay. While the fluorescence-based measurements require an independent measurement to assess activity of a given serum against each virus, this system was developed to perform massively parallel neutralization assays with the barcoded influenza variant libraries using next generation sequencing (NGS). As shown in FIG. 10A-10B, similar measurements for strains are obtained using the NGS-based neutralization assay method in comparison with fluorescence-based measurements. However, with the NGS-based neutralization assay, neutralization potency against all variants included in the library can be obtained with a single dilution series. Analysis of these data allows calculation of IC50-like measurements for multiple viruses at once, using the same volume of sample that is currently used to generate an IC50 against a single virus. This advancement allows generation of significantly more measurements than prior systems, allowing for the extraction of more detailed information about the immune specificity of a given sample against many viruses, even for samples which have limited volume can be gained. Absolute measurements of viral neutralization can also be generated in high-throughput mode. Taken together, the disclosed barcoded influenza viruses and resulting mutational scanning libraries provide an important advance in the ability to generate, store, and characterize a large number of variant viral proteins including influenza.

For example, the methods described herein may allow for the assessment of multiples of strains simultaneously, for example, 50-100 strains. Assessing these strains against historical libraries may reveal mutations that are responsible for escaping existing immunity of an individual. Assessing a multitude of strains against recent strain libraries could be used as part of surveillance measure to determine which recent trains are most antigenically distinct and therefore inform which clades might be likely to grown in frequency of the next influenza season. Assessing a multitude of strains against combinatorial libraries allows for the assessment of the interaction between mutations for a given function. Assessing a multitude of strains against focused mutational libraries would allow one to assess the impact of many mutations within a specific region of a protein or within a single or combination of epitopes.

The influenza virus belongs to the Orthomyxoviridae family and is an enveloped virus with an eight-segmented single-stranded, negative-sense viral RNA (vRNA) genome. Influenza virions (the complete, infective form of a virus outside a host cell, with a core of RNA and a capsid) enter the host cell, where their negative sense RNA is released into the cytoplasm. The virus' own RNA replicase, known as RNA-dependent RNA polymerase (RdRp), is used to form positive sense RNA template strands through complementary base pairing. There are two distinct forms of this positive sense RNA: one that serves as messenger RNA (mRNA), which is translated into viral proteins by ribosomes of the host cell; and another that serves as template to make more negative sense RNA strands.

Using long read sequencing, barcodes may be linked to the gene sequence of any variant of interest. Once the linkage between the barcode and the gene sequence has been completed, variants that infect cells may be efficiently identified using short read sequencing of the viral RNA. The chimeric barcoded segment may be used to incorporate either viral genes of interest or non-neutralizable virus standards. In addition, the same construct design may be used to generate nucleic acid standards which resemble the vRNA of the virus of interest, for example, influenza, and can be added during or after nucleic acid extraction to allow for normalization of sequencing counts per variant between conditions.

The size of the duplicated packaging signal of a particular virus is variable. For example, depending on the particular influenza virus strain and genome segment being modified, the duplicated packaging signal sequences include 50-200 nucleotides (Gerber, et al., Trends Microbiol. 22:446-455 (2014); Hutchinson, et al., J. Gen. Virol. 91:313-328 (2010)). For example, the packaging signal for NP vRNA of influenza A includes 120 nucleotides at the 5′ end, in addition to the noncoding regions (Ozawa, et al., J. Virol 81:30-41 (2006)). Packaging signals for other influenza A virus segments have also been identified (Gao, et al., J. Virol. 86:7043-7051 (2012)). SEQ ID NOs. 1 and 3 provide exemplary packaging signals for the 5′ end for Influenza A virus Segment 4, and the 5′ end for Influenza A virus Segment 6 respectively. As will be understood by one of ordinary skill in the art, a packaging signal can refer to the shortest sequence required to allow packaging of vRNA. In particular embodiments, the packaging signal of a virus includes 50 nucleotides, 60 nucleotides, 70 nucleotides, 80 nucleotides, 90 nucleotides, 100 nucleotides, 110 nucleotides, 120 nucleotides, 130 nucleotides, 140 nucleotides, 150 nucleotides, 160 nucleotides, 170 nucleotides, 180 nucleotides, 190 nucleotides, or 200 nucleotides from the 5′ end of a vRNA genome segment. In particular embodiments, the packaging signal includes 50 nucleotides-60 nucleotides, 60 nucleotide 70 nucleotides, 70 nucleotides-80 nucleotides, 80 nucleotides-90 nucleotides, 90 nucleotides-100 nucleotides, 100 nucleotides-110 nucleotides, 110 nucleotides-120 nucleotides, 120 nucleotides-130 nucleotides, 130 nucleotides-140 nucleotides, 140 nucleotides-150 nucleotides, 150 nucleotides-160 nucleotides, 160 nucleotides-170 nucleotides, 170 nucleotides-180 nucleotides, 180 nucleotides-190 nucleotides, or 190 nucleotides-200 nucleotides from the 5′ end of the vRNA genome segment. In particular embodiments, a range of nucleotides for a packaging signal from the 5′ end of a vRNA genome segment includes a portion of coding region of a vRNA genome segment and a portion of non-coding region adjacent to the coding region.

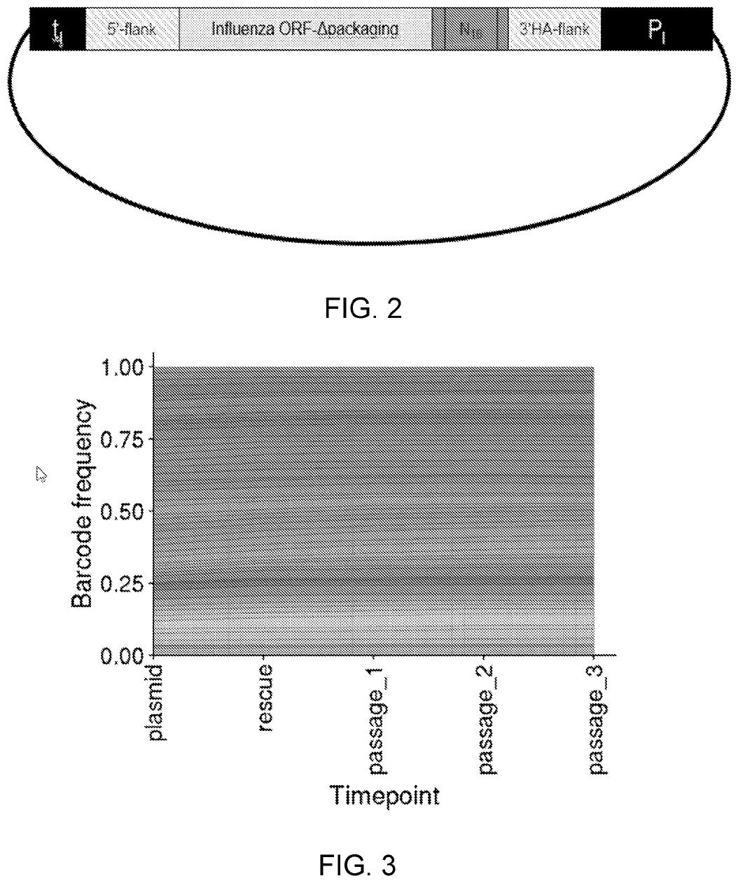

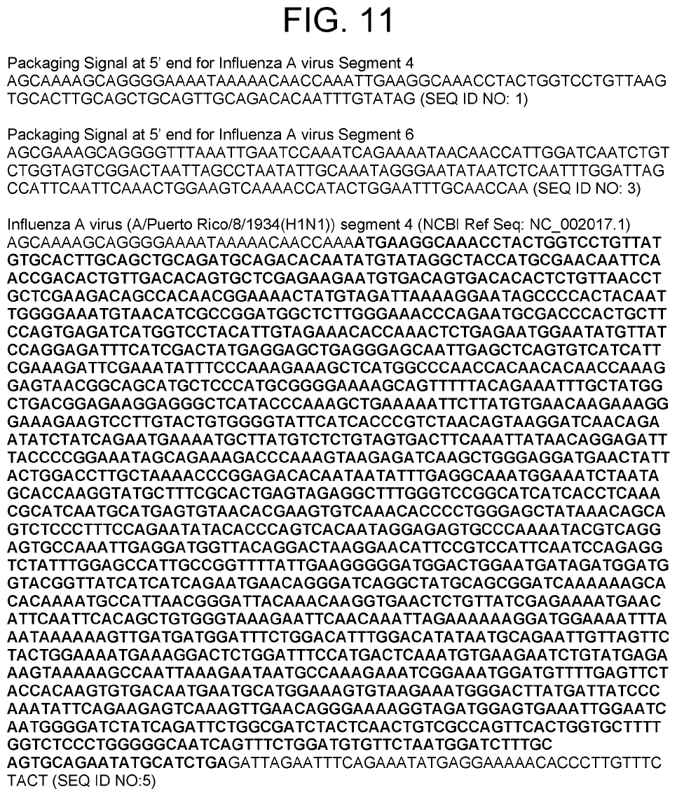

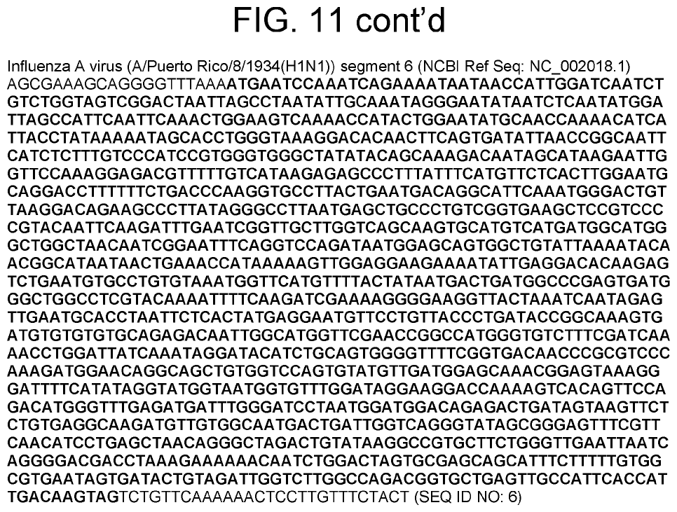

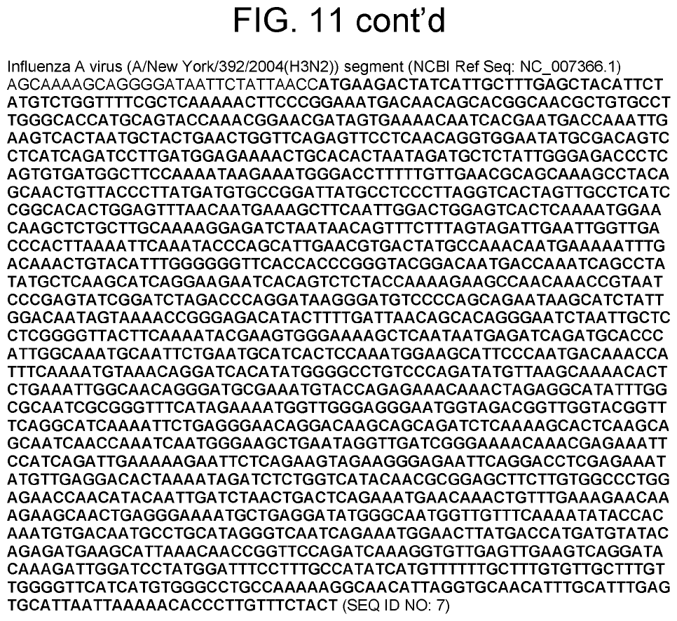

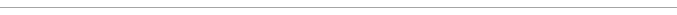

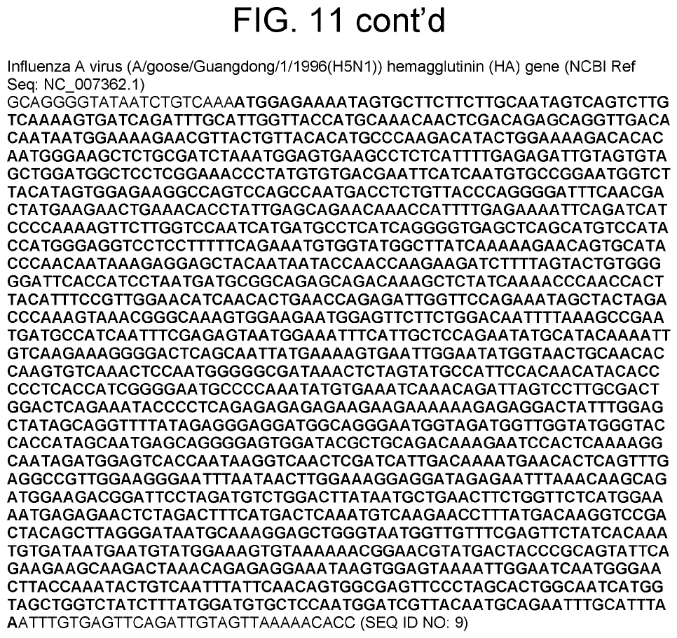

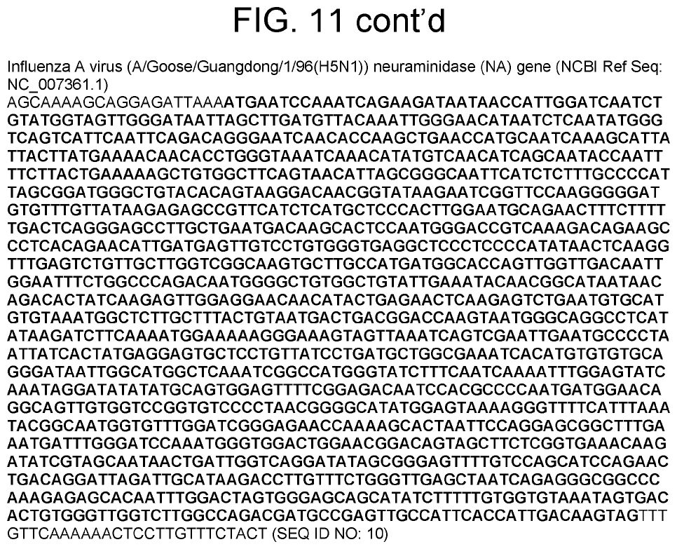

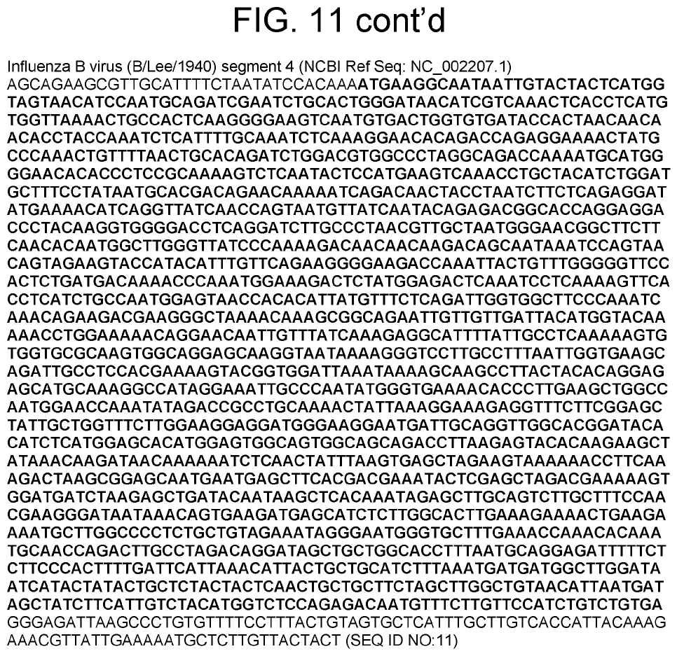

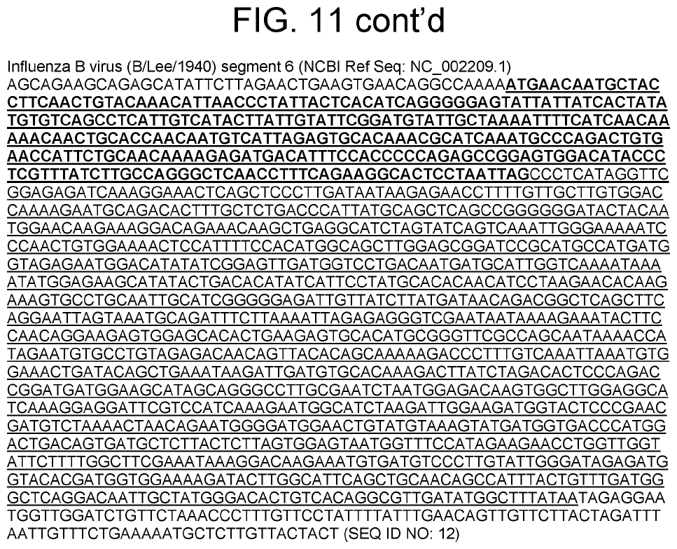

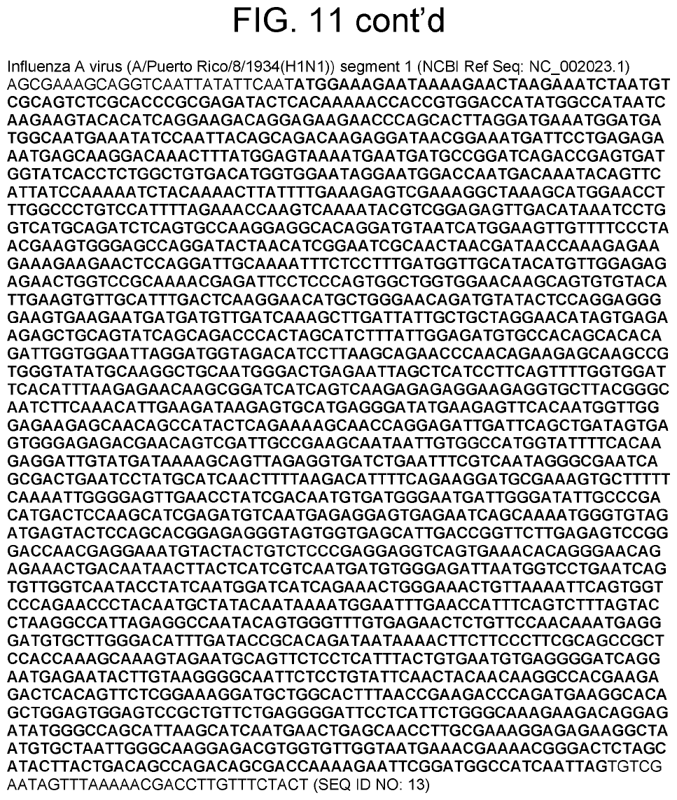

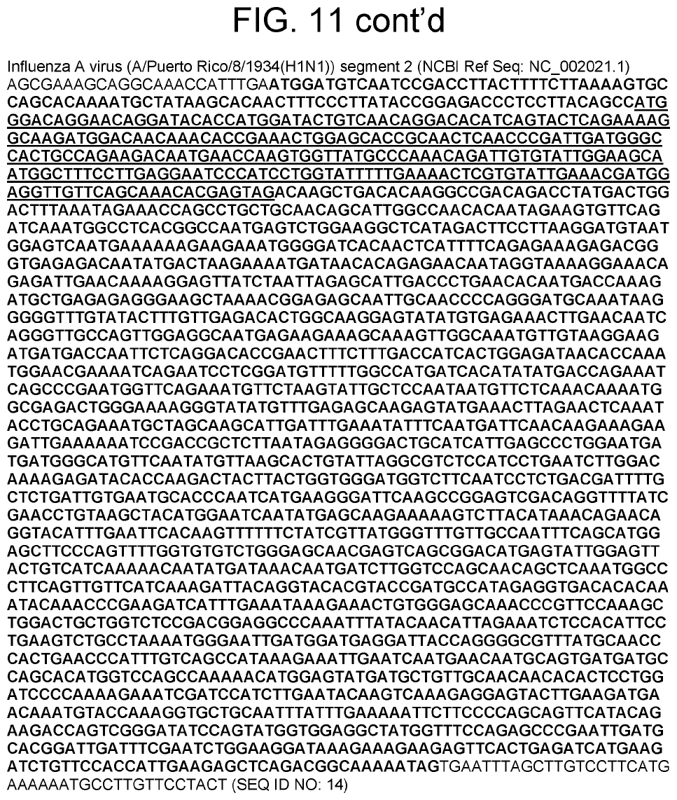

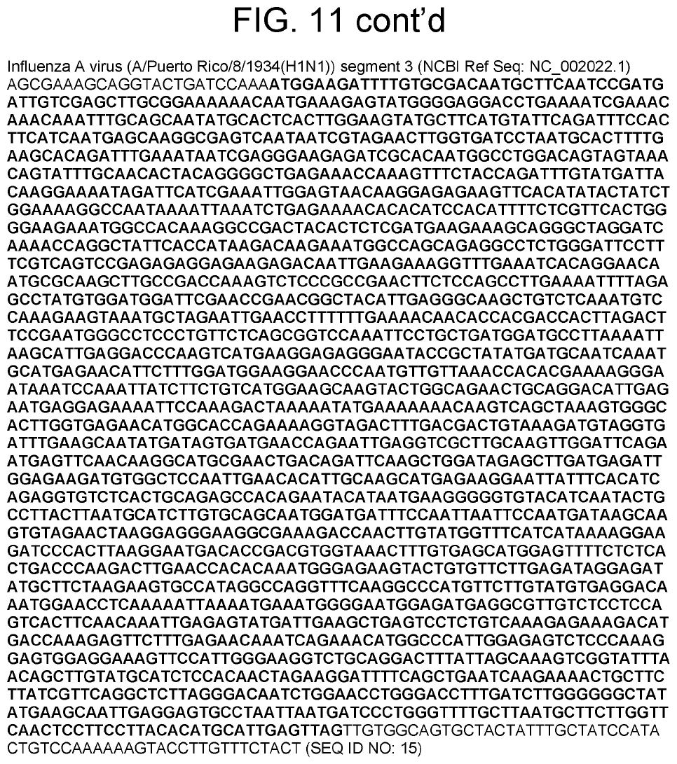

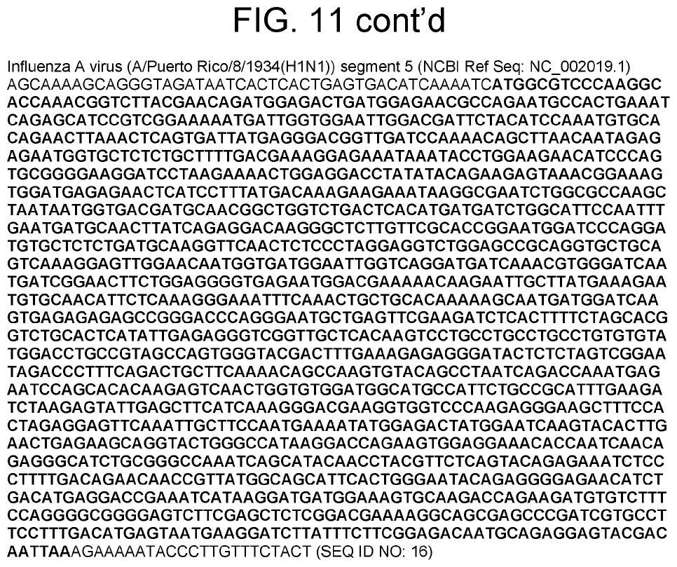

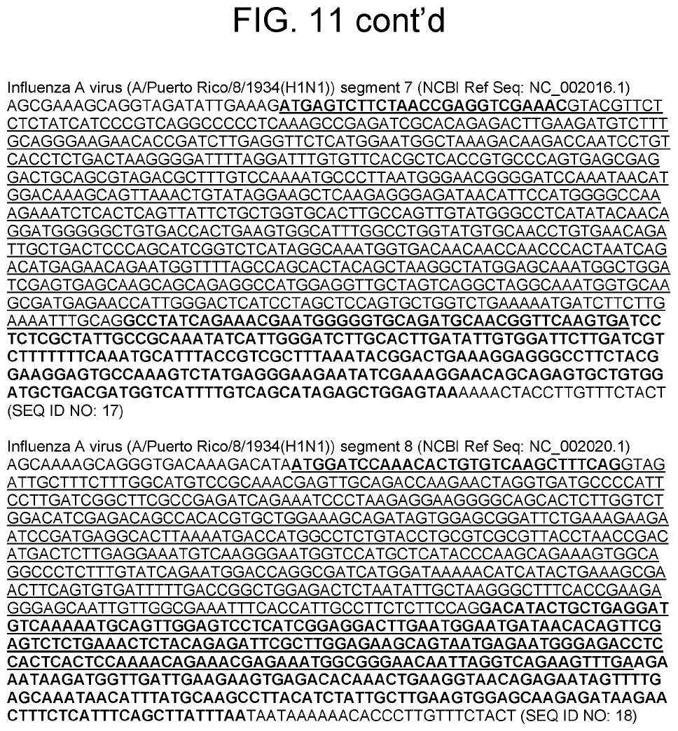

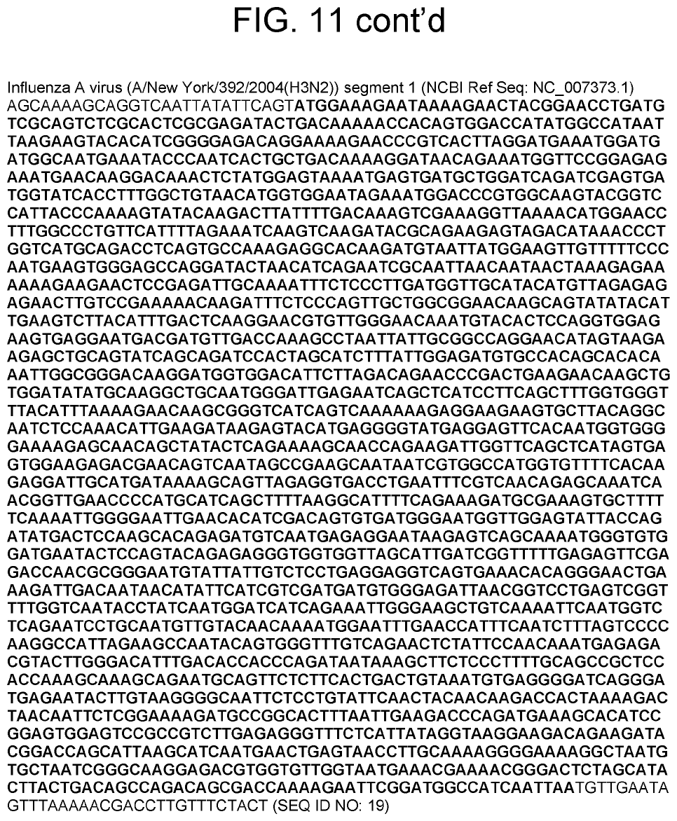

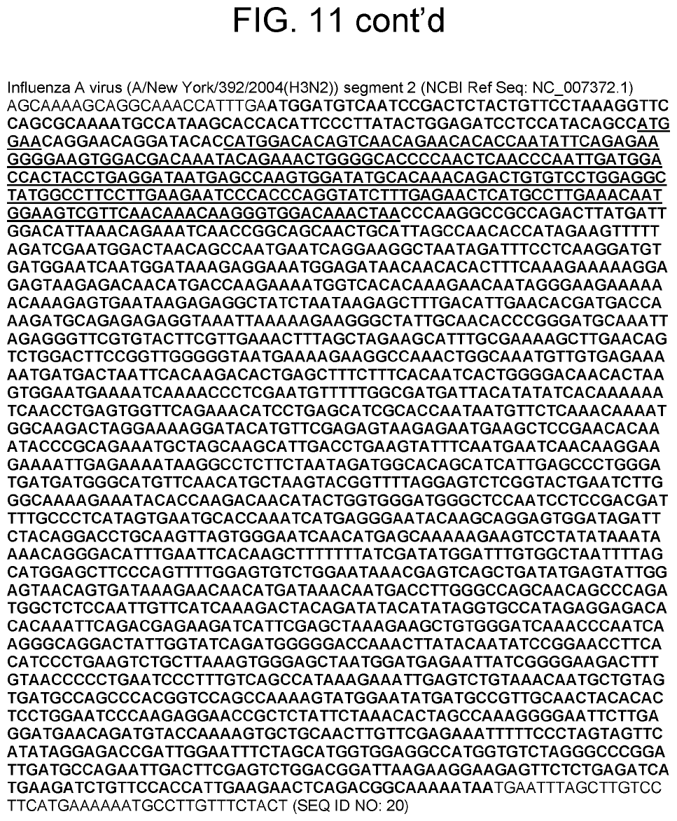

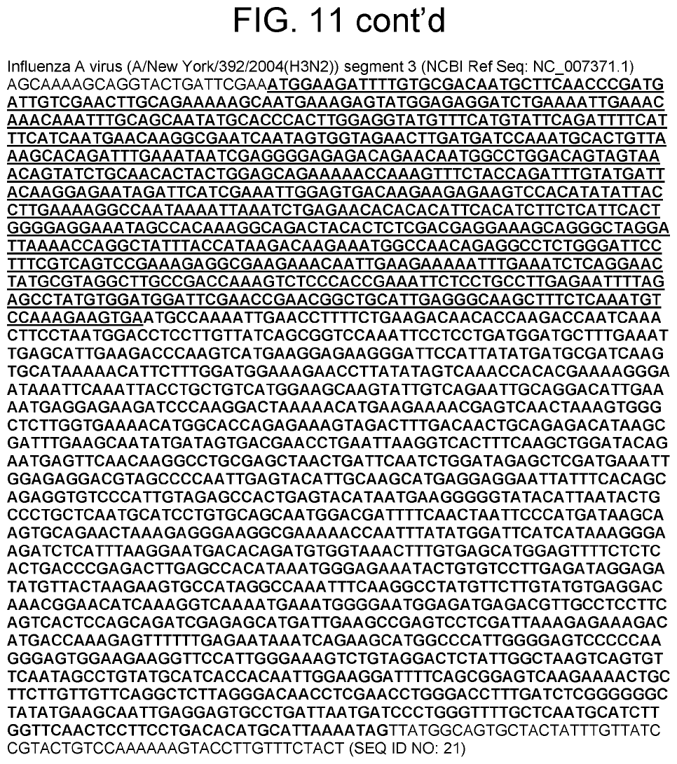

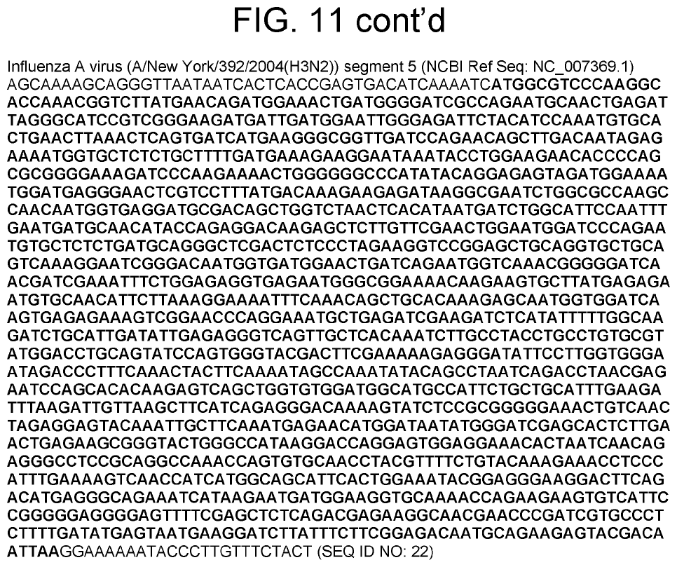

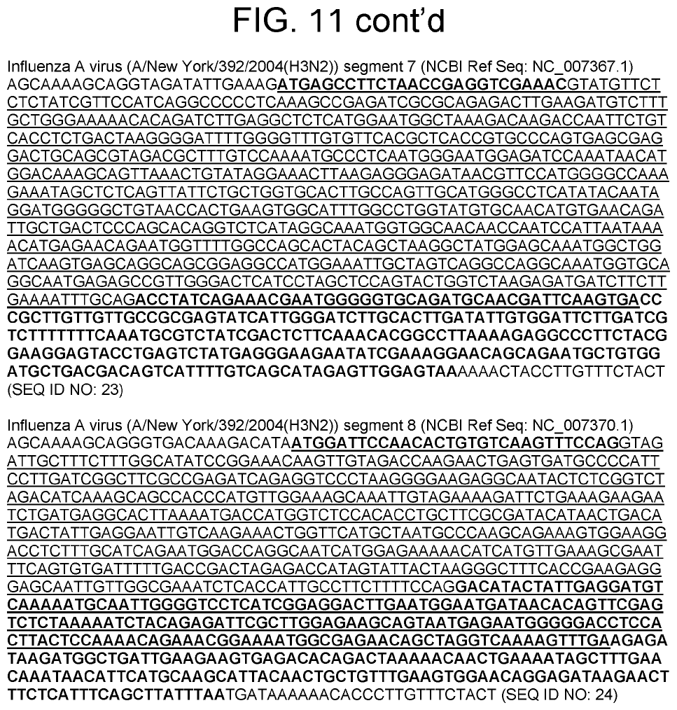

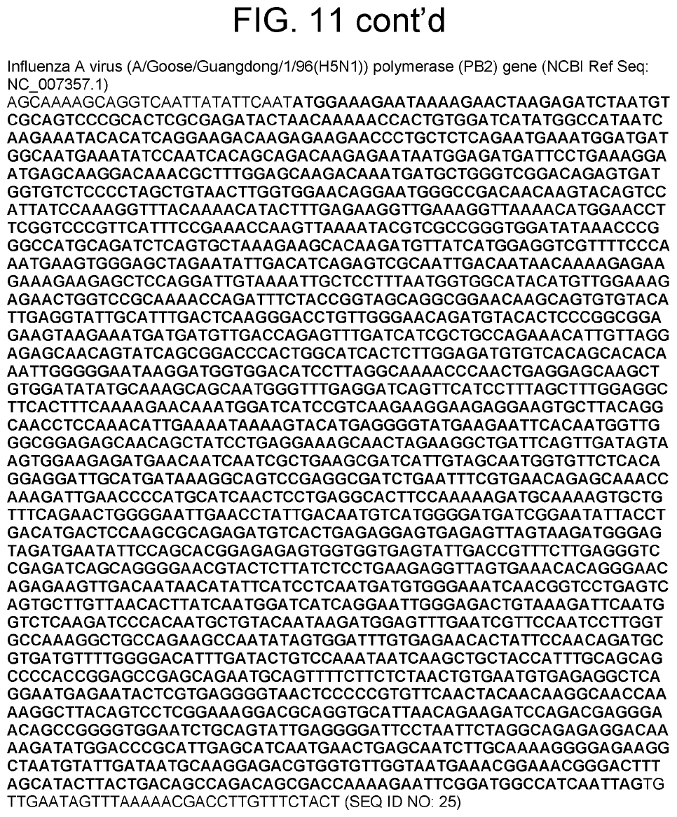

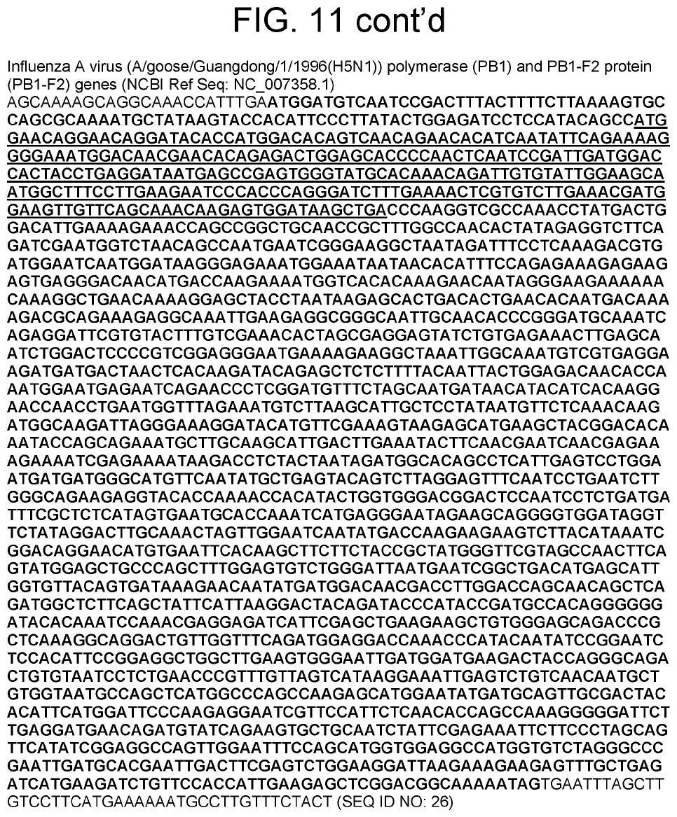

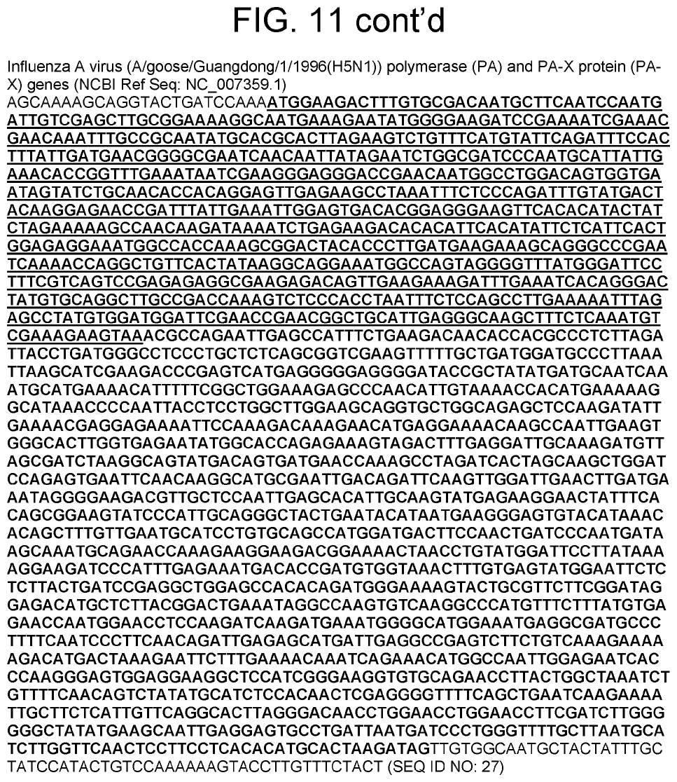

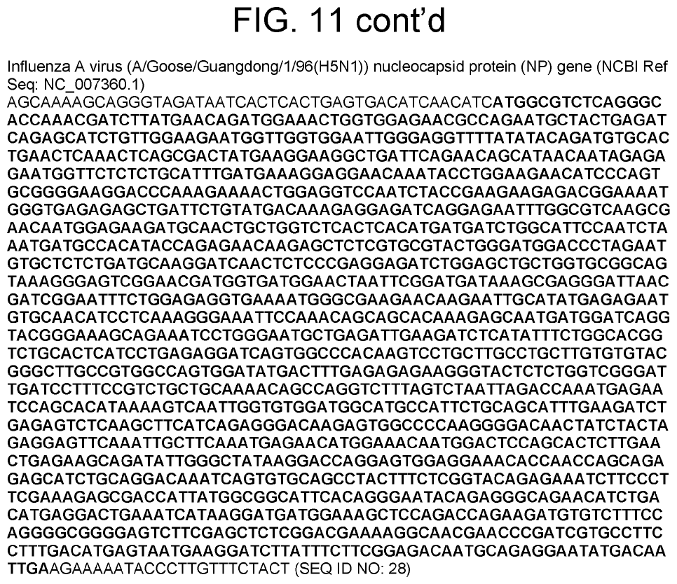

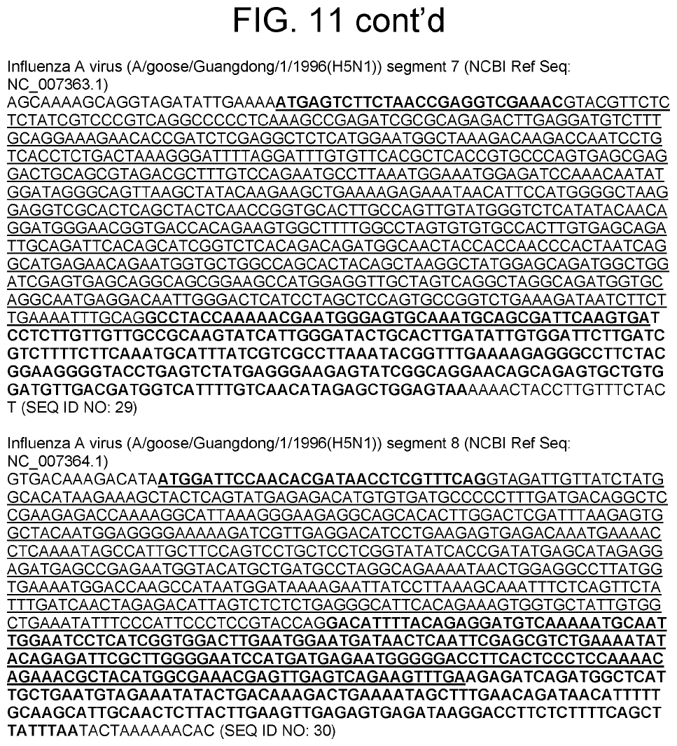

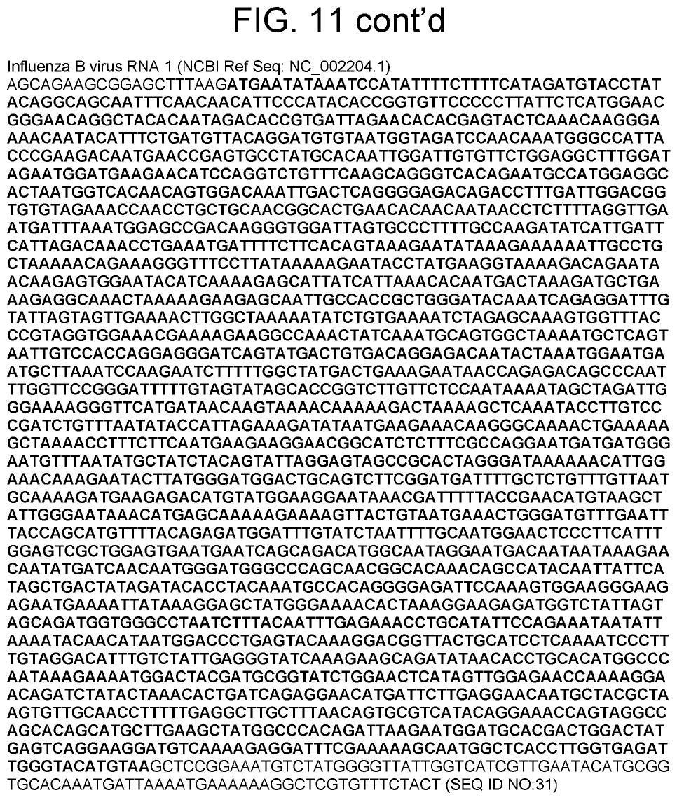

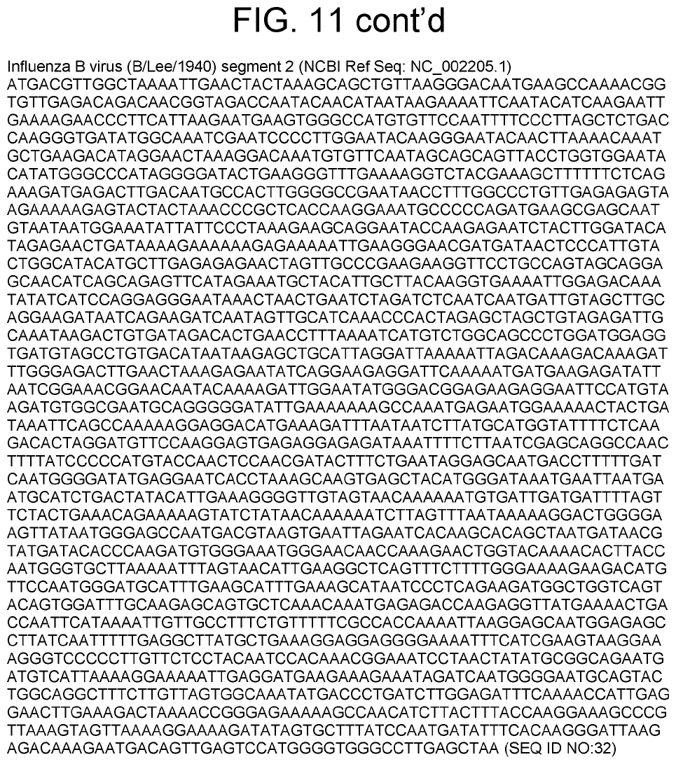

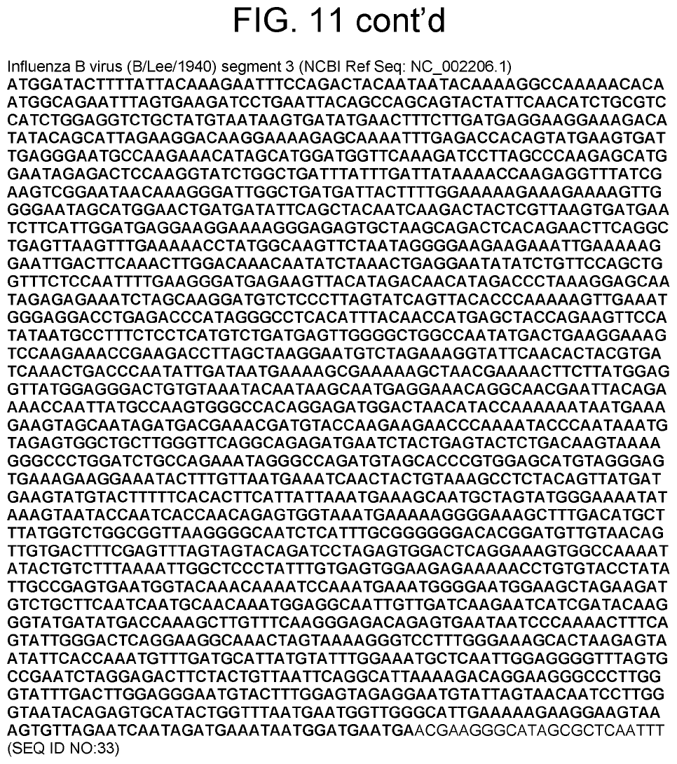

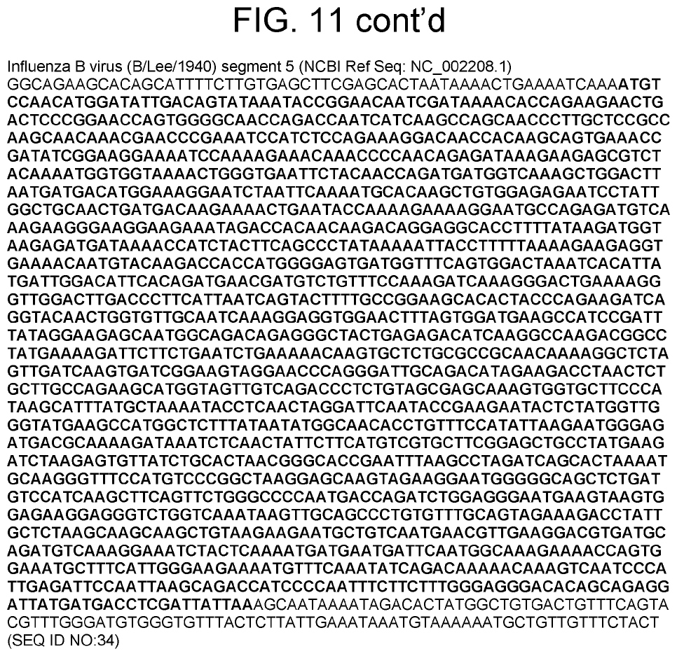

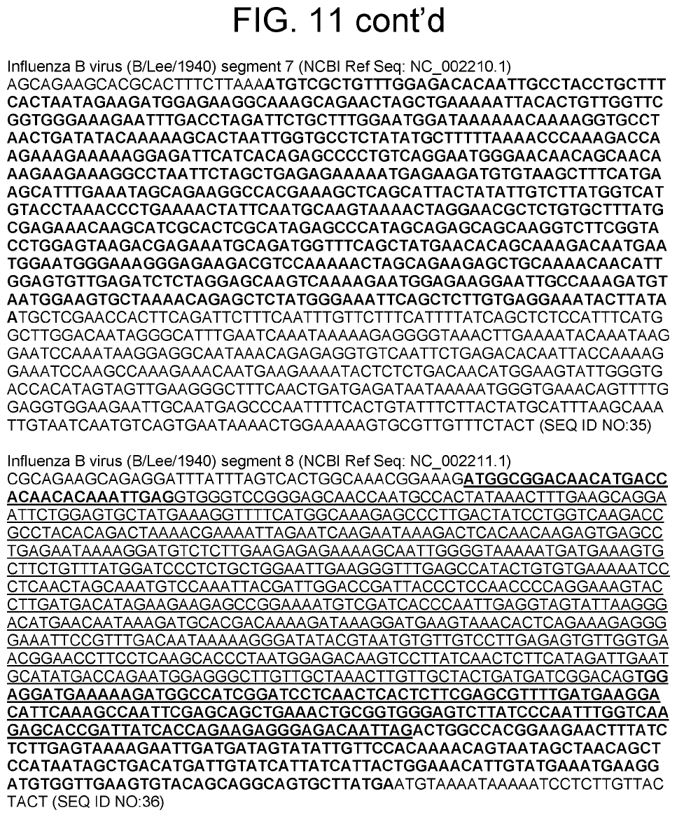

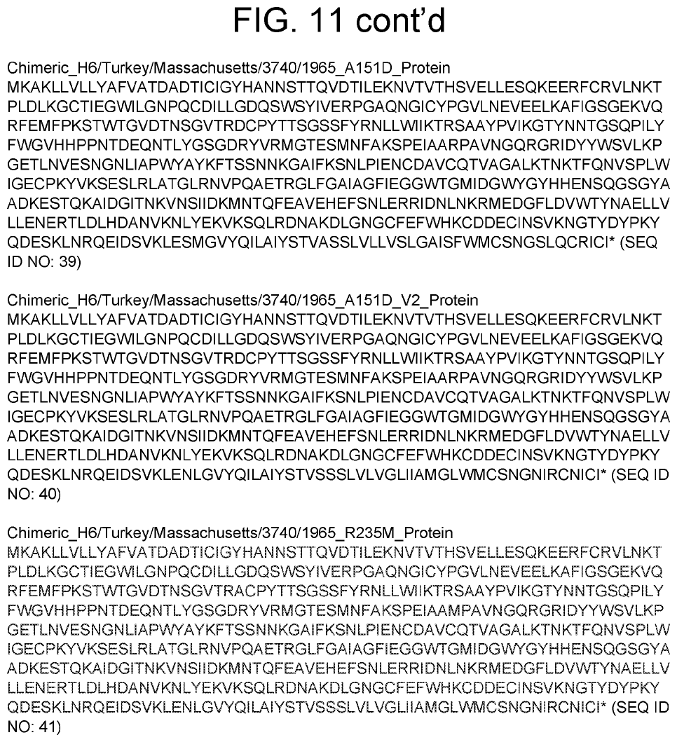

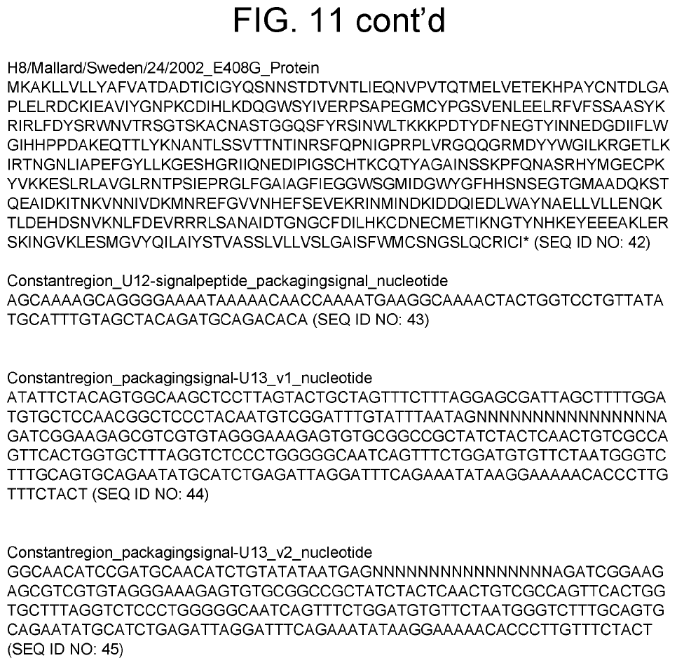

As indicated, the barcode of the systems and methods disclosed herein is inserted between the end of the viral genome segment's open reading frame (ORF) (corresponding to the stop codon of the transcribed positive sense mRNA) and the inserted copy of the 5′ vRNA packaging signal. Exemplary ORF coding sequences are depicted in FIG. 11, SEQ ID NOs. 5-36. These sequences provide guidance regarding ORFs, the start and stop codons of the coding sequences, non-coding regions 5′ and 3′ of an ORF, and exemplary packaging signals. FIG. 2 depicts an exemplary plasmid barcoded.

Exemplary plasmids can be derived from cloning plasmids such as pUC18 or pUC19 plasmids (Norrander et al. Gene. 1983 December; 26 (1): 101-106). Exemplary plasmids include plasmids that allow transcription of negative sense vRNA from each of the eight genomic segments of influenza virus (FIG. 2). In particular embodiments, the plasmids can include a promoter, a barcoded vRNA genome segment, and a terminator sequence. In particular embodiments, the promoter in the plasmid can include a truncated human RNA polymerase I promoter, for example, the truncated human RNA polymerase I promoter of GenBank SEQ ID: M13001. A truncated human RNA polymerase I promoter includes nucleotides −250 to −1 of the human polymerase I promoter. In particular embodiments, the barcoded vRNA genome segment in a plasmid is oriented such that transcription from the promoter results in production of negative sense vRNA genome segments. A barcoded vRNA genome segment in a plasmid includes barcoded, double stranded complementary DNA (cDNA) that has been reverse transcribed and amplified from the negative sense vRNA genome segment. In particular embodiments, a barcoded vRNA genome segment in a plasmid includes non-coding regions 5′ and 3′ to the coding region of the vRNA genome segment. Transcription plasmids include a terminator sequence to ensure that the transcribed positive sense mRNA has a proper 3′ end. In particular embodiments, the terminator sequence can be derived from a hepatitis delta virus ribozyme sequence or a mouse RNA polymerase I terminator.

In particular embodiments, exemplary plasmids can also include plasmids that allow expression of a set of viral proteins required for encapsidation, transcription, and replication of the viral genome. The set of viral proteins required for encapsidation, transcription, and replication of the viral genome includes the three subunits of the viral RNA-dependent RNA polymerase complex (PB1, PB2, and PA) and the nucleoprotein (NP). Expression plasmids can include a promoter to drive expression of PB1, PB2, PA, and NP proteins encoded by corresponding cloned cDNA. PB1, PB2, PA, and NP proteins can amplify and transcribe (into mRNA) the negative sense vRNA produced from the plasmids described above. Promoters that can drive expression of PB1, PB2, PA, and NP proteins include mouse hydroxymethylglutaryl-coenzyme A reductase (HMG) promoter, adenovirus type 2 major late promoter, the cytomegalovirus (CMV) promoter, and chicken β-actin promoter.

In particular embodiments, exemplary plasmids of the present disclosure can be ambisense expression plasmids. Ambisense expression plasmids are bidirectional plasmids that allow both transcription of a negative sense vRNA and expression of the recombinant viral protein encoded by the ORF from that vRNA. In particular embodiments, an ambisense plasmid can include cDNA that has been reverse transcribed and amplified from a negative sense vRNA genome segment. In particular embodiments, an ambisense plasmid can include non-coding regions 5′ and 3′ to the coding region of the vRNA genome segment. In one direction of the plasmid, a polymerase I transcription cassette (e.g., viral cDNA between human RNA polymerase I promoter and a mouse terminator sequence) allows production of negative sense vRNA. In the opposite direction, a polymerase II transcription cassette (viral cDNA between chicken β-actin promoter and polyA) encodes the viral protein encoded by the same vRNA genome segment. An example of an ambisense plasmid is described in Martinez-Sobrido and Garcia-Sastre J Vis Exp. 2010; 42:2057. Transfection of appropriate plasmids into a cell line allows intracellular reconstitution of ribonucleoprotein complexes that include barcoded genome segments for production of barcoded influenza viruses.

An exemplary protocol for transfection of plasmids containing barcoded vRNA genome segments of the present disclosure is briefly described. A plasmid transfection mixture including appropriate media (e.g., Opti-MEM™ media, Thermo Fisher Scientific, Waltham, MA), plasmids containing barcoded vRNA genome segments, and a transfection agent (e.g., Lipofectamine) can be prepared. The plasmid transfection mixture can then be incubated with cell lines to be transfected (e.g., 293T and/or MDCK cells) for a period of time (e.g., overnight) under appropriate conditions (e.g., 37° C. and 5% CO₂). The media can be changed during the transfection period. Supernatant from transfected cells can be used to infect fresh cell lines (or chicken embryonated eggs) for a period of time (e.g., 37° C. for 2 to 3 days). For cell lines, a cytopathic effect can be seen at a period of time (e.g., 48-72 hours) after passage of the cells and can suggest successful rescue of barcoded virions. Assays such as hemagglutination (HA) assays and/or immunofluorescence assays can be performed to detect the presence of rescued virus in cell culture supernatant or in the allantoic fluid of harvested eggs. In an HA assay, the presence of virus induces hemagglutination of red blood cells, while the absence of virus allows the formation of a red pellet in the bottom of the well. Immunofluorescence assays can make use of sera that recognize a viral antigen and fluorescently labeled secondary antibodies. Once an assay identifies the presence of rescued virus, the virus can be plaque purified, and the genetic composition of the virus can be confirmed by RT-PCR and sequencing.

The barcoded influenza viruses described herein can be used to create deep mutational scanning libraries for the study of influenza virus proteins. Within these libraries, in particular embodiments, each variant carries a unique barcode. The selectively neutral barcodes can be linked to the viral mutations by long-read sequencing. Thereafter, the functional and antigenic effects of viral mutations (both singly and in combination) can be easily read out by sequencing the barcodes. This approach greatly improves the power and accuracy of deep mutational scanning of influenza virus genes.

Within the current disclosure, “selectively neutral” and “with minimal to no effects on viral fitness” can be used interchangeably. As used herein, selectively neutral means the mutation inferred no advantage or disadvantage on the virus. In some aspects, selectively neutral may mean a low selection coefficient. The selective neutrality of barcoding can be validated by creating a pool of viruses with different barcodes and passaging them at least two times in cell culture to demonstrate that no barcode increases or decreases in frequency by more than 2-fold after correcting for statistical sampling error (see, e.g., FIG. 3). While the influenza virus will be used as an exemplary virus, the methods described herein may also be applied to other viruses of interest.

Variant libraries generated using methods disclosed herein have numerous applications. In particular embodiments, the systems and methods disclosed herein can be used to map the epitopes of influenza-virus binding antibodies; to inform antibody drug development by characterizing mutations in target viral proteins that allow development of influenza resistance to antibodies; de novo structure prediction; homology modeling; structure determination; and/or to assess the ability of different influenza virus entry proteins to evade antibody neutralization, overcome drug inhibition, and/or infect new species. In some aspects, the potential contagiousness and transmissibility (R₀) of viruses may be evaluated. For example, the R₀ of influenza is generally between 1 to 2, however, the R₀ of the 1918 version of influenza was 2.8. Measles has an R₀ of 12-18.

If numerous mutations to the viral entry protein allow antibody evasion, drug resistance, and/or infection of a new host species, the viral strain may have a higher probability of becoming a health threat. If, however, only few or very specific mutations allow antibody evasion, drug resistance, and/or infection of a new host species, the viral strain may pose less of a threat.

In particular embodiments, deep mutational scanning combines functional selection with high throughput sequencing to measure the effects of mutations on protein function. In particular embodiments, a library of 10⁴ to 10⁵ variants of a given protein is constructed and selection for function is imposed. Under modest selection pressure, variant frequencies are perturbed according to the function of each variant. Variants harboring beneficial mutations increase in frequency, whereas variants harboring deleterious mutations decrease in frequency. In particular embodiments, high throughput sequencing can be used to measure the frequency of each variant during the selection experiment, and a functional score can be calculated from the change in frequency over the course of the experiment. In particular embodiments, the result is a largescale mutagenesis data set containing a functional score for each variant in the library. Fowler et al. Nature Protocols 9:2267-2284 (2014). As one example, in particular embodiments, sera samples can be obtained from vaccine studies to map mutations that affect resistance to these sera. This work can functionally map the epitopes targeted by the vaccines and enable correlation of animal-to-animal variation in protection with variation in epitope targeting, both of which could help inform further immunogen design.

The deep mutational scanning libraries disclosed herein can also include absolute standards. These absolute standards can be based on viruses with glycoproteins that are not recognized by a species of interest. For example, in particular embodiments, the absolute standards can be based on viruses with glycoproteins from influenza strains other than human influenza strains that are not recognized by human sera or antibodies. That is, they do not react with human sera. With the inclusion of such absolute standards, selection on mutations can be quantified in high-throughput mode.

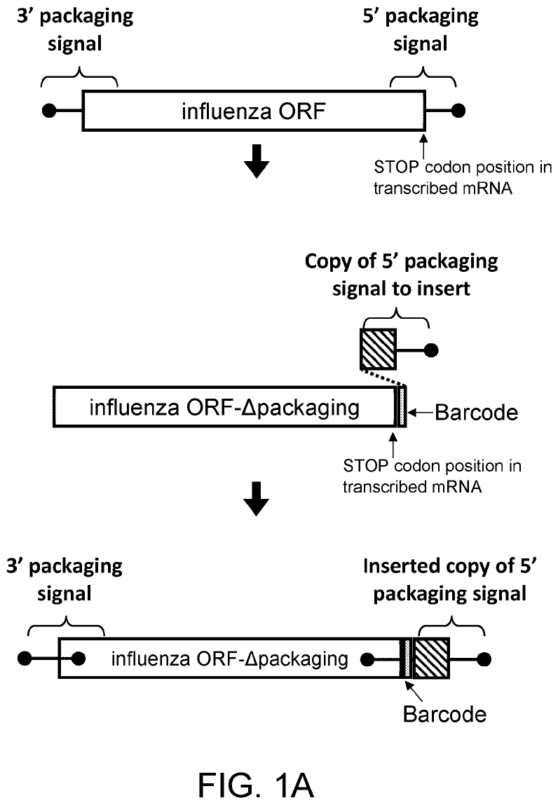

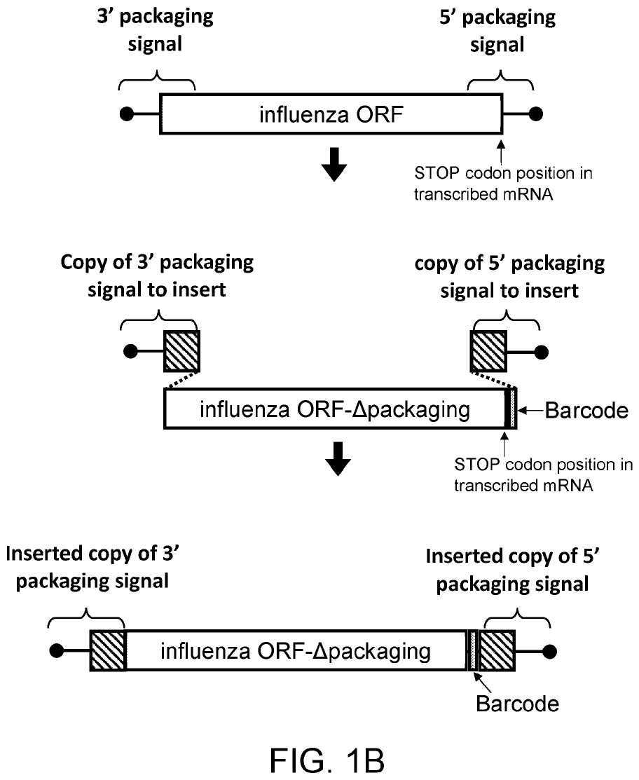

Systems and methods disclosed can be used to successfully create a barcoded deep mutational scanning library of the hemagglutinin segment with >200,000 unique barcodes per library. Similar methods may be used to generate a neuraminidase library. Libraries of barcoded wild type hemagglutinin (HA) gene segments have also been generated with 50 to >1 million barcodes. For example, FIG. 1 depicts barcoded influenza virus vRNA with packaging signals decoupled from the coding sequence. In particular embodiments, the barcode is inserted along with a copy of the coding region of a 5′ viral RNA genome packaging signal between a terminus of a corresponding genome segment open reading frame and a naturally occurring noon-coding portion of the 5′ viral RNA genome packaging signal. In FIG. 1A, a sufficient sequence of the 5′ end of the viral RNA (which is the 3′ end of the mRNA transcribed from the negative sense vRNA depicted in the FIG.) is duplicated (typically >90 nucleotides). This duplicated sequence is inserted before the non-coding portion of the 5′ endogenous packaging signal with a barcode inserted between the terminus of the viral protein-coding region and the duplicated/inserted packaging signal. The duplicated sequence typically includes noncoding and coding sequences to capture the packaging signal. FIG. 1B depicts the approach shown in FIG. 1A and additionally performing a similar duplication and insertion at the 3′ end of the gene segment. In particular embodiments, duplication and insertion at the 3′ vRNA end is not used and is expressly excluded. The barcode is inserted along with a copy of the coding region of a 5′ viral RNA genome packaging signal between a terminus of a corresponding genome segment open reading frame and a naturally occurring noon-coding portion of the 5′ viral RNA genome packaging signal. FIG. 2 depicts an exemplary plasmid barcoded according to methods of the current disclosure. As shown in FIG. 3, the barcodes did not affect viral fitness.

Aspects of the current disclosure are now described with additional detail and options as follows: (i) Influenza Virus; (ii) Barcoded Deep Mutational Scanning Libraries; (iii) Exposure to Selection Pressures; (iv) Engineering More Effective Antibodies; (v) Vaccine Selection; (vi) Selection of Effective Anti-Viral Conditions and/or Effective Therapeutic Compounds; (vii) Host Adaptation Studies; (viii) Kits; (ix) Exemplary Embodiments; (x) Experimental Example; and (xi) Closing Paragraphs. As will be understood by one of ordinary skill in the art, information within each of the disclosure sub-headings can apply to information within other sub-headings, and the sub-headings are provided only for organizational convenience.

(i) Influenza Virus. The influenza virus belongs to the Orthomyxoviridae family, which are enveloped viruses with single-stranded, negative-sense RNA genomes. The types of influenza viruses include: influenza A virus, influenza B virus, influenza C virus, and influenza D virus.

Influenza A viruses can infect humans and a variety of animals, such as pigs, horses, marine mammals, cats, dogs, and birds and therefore pose a significant risk of zoonotic infection, host switch, and the generation of pandemic viruses. Some well-known flu pandemics include: the 1918 H1N1 Spanish flu, the 1957 H2N2 Asian flu, the 1968 H3N2 Hong Kong flu, and the 2009 H1N1 swine flu (Shao, et al., Int. J. Mol. Sci. 18 (8): 1650 (2017)). Influenza C is associated with mild respiratory illness and is not thought to cause epidemics or pandemics. Thus far, influenza D viruses have only been found to affect swine and cattle and therefore are not known to cause illness in humans.

The influenza A virus and influenza B virus have an eight-segmented viral RNA (vRNA) genome, whereas influenza C virus has a seven-segmented vRNA genome. Although recently isolated, influenza D virus is also believed to have a seven-segmented vRNA genome (Nakatsu, et al., J. Virol 92(6): e02084-17 (2018)). Despite this, Nakatsu, et al. found that influenza viruses, including influenza C virus and influenza D virus, package eight ribonucleoprotein complexes (RNPs) regardless of RNA segments in their genome. These vRNA segments encode viral proteins.

The influenza A virus genome is 13 kb and encodes 13 proteins (Jagger et al., Science. 337:199-204 (2012)) including: hemagglutinin (HA), neuraminidase (NA), M1 matrix protein (M1), M2 ion channel protein (M2), nuclear protein (NP), nonstructural protein (NS1, NS2 (NEP)), and RNA polymerase complex (PB1, PB2, PA) (Cox et al., 2000 Annu. Rev. Med. 51:407-421). Additional viral proteins expressed by splicing, alternative initiation, or ribosomal frameshifts from the eight segments include PB1-F2, PB1-N40, and PA-X (Muramoto et al. Journal of Virology 2013; 87(5): 2455-2462. The influenza B virus differs in that instead of an M2 protein, it has a BM2 protein and has a viral segment with both NA and NB sequences.

Influenza A viruses can be divided into subtypes on the basis of their surface glycoproteins, HA and NA. There are 18 HA subtypes and 11 NA subtypes. Influenza A viruses can be further classified by strains, such as the influenza A (H1N1) and influenza A (H3N2) viruses. Influenza B and C viruses can be classified by lineage or by strains (Hay et al., Philos. Trans. R. Soc. Lond. B. Biol. Sci. 356:1861-1870 (2001); Aoyama, et al., Virology. 1991; 182:475-485 (1991)).

The influenza A genes encoding the viral surface proteins, HA and NA, that form the main targets of neutralizing antibodies, are critical for the evolution of the virus. All known influenza A viruses have been found in birds, except subtypes H17N10 and H18N11 which have only been found in bats. Human influenza A viruses have only been detected with the subtypes of HA, including H1, H2, H3, H5, H6, H7, H9, and H10 and subtypes of NA, including N1, N2, N6, N7, N8, and N9. In swine, the detected HA subtypes include: H1, H2, H3, H4, H5, and H9 with the detected NA subtypes including: N1 and N2. Other animals have been found with the HA subtypes: H3, H4, and H7 and NA subtypes N7 and N8.

The life cycle of influenza virus can be briefly described as follows. Influenza virions (the complete, infective form of a virus outside a host cell, with a core of RNA and a capsid) enter the host cell, where their negative sense RNA is released into the cytoplasm. The virus' own RNA replicase, known as RNA-dependent RNA polymerase (RdRp), is used to form positive sense RNA template strands through complementary base pairing. There are two forms of positive sense RNA: one serves as messenger RNA (mRNA), which is translated into viral proteins by ribosomes of the host cell; the other serves as template to make more negative sense RNA strands. In viruses with segmented genomes like influenza virus, replication occurs in the nucleus and the RdRp produces one monocistronic mRNA strand (encoding one polypeptide per RNA molecule) from each genome segment. New viral capsids are assembled with the capsomere proteins. The negative sense RNA strands combine with capsids and viral RdRp to form new negative sense RNA virions. After assembly and maturation of nucleocapsid, the new virions exit the cell through the cell membrane by budding or lysis to further infect other cells.

The influenza genome is packaged into progeny virions by cis-acting, segment-specific packaging signals found on each vRNA. These packaging signals include bipartite sequences at the 5′ and 3′ ends of the vRNA, which house not only conserved promoter sequences but also coding and segment-specific non-coding regions adjacent to the promoter region. Each packaging signal is unique to each vRNA, and it has been shown that the 5′ sequence is more important than the 3′ sequence for genome packaging, and that a longer 5′ sequence is better for genome packaging. In addition, studies have shown that nucleotide length is important, but the actual sequence is less so (random sequences are sufficient to generate viruses).

As indicated previously, representative packaging signal sequences and genome segments are provided in FIG. 11 as SEQ ID NOs. 1, 3, and 5-36.

(ii) Barcoded Deep Mutational Scanning Libraries. Barcoded deep mutational scanning libraries described herein include barcoded influenza virus. In particular embodiments, a deep mutational scanning library includes influenza protein variants with 19 possible amino acid substitutions at each amino acid position and all possible codons of the associated 63 codons at each amino acid position of an influenza viral protein under analysis. In particular embodiments, a deep mutational scanning library includes influenza protein variants with every possible codon substitution at every amino acid position in a gene of interest with one codon substitution per library member. A deep mutational scanning library can also include variants with one, two, or three nucleotide changes for each codon at every amino acid position in a gene of interest with one codon substitution per library member. A deep mutational scanning library can also include variants with one, two, or three nucleotide changes for each codon at two amino acid positions, at three amino acid positions, at four amino acid positions, at five amino acid positions, at six amino acid positions, at seven amino acid positions, at eight amino acid positions, at nine amino acid positions, at ten amino acid positions, etc., up to at all amino acid positions, in a gene of interest with one codon substitution per library member. In particular embodiments, the start codon is not mutagenized. In particular embodiments, the start codon is methionine (Met).

In particular embodiments, a deep mutational scanning library includes variants with one, two, or three nucleotide changes for each codon at every amino acid position in a gene of interest with more than one codon substitution, more than two codon substitutions, more than three codon substitutions, more than four codon substitutions, or more than five codon substitutions, per library member. In particular embodiments, a deep mutational scanning library includes variants with one, two, or three nucleotide changes for each codon at every amino acid position in a gene of interest with up to all codon substitutions per library member.

In particular embodiments, 20% of library members can be wildtype, 35% can be single mutants, and 45% can be multiple mutants. Multiple mutants can be advantageous, and the sequencing required by the systems and methods disclosed herein is so efficient that using 20% of reads on wildtype is not a problem. Additionally, there are alternative (more complex) mutagenesis methods that give a larger proportion of single amino acid mutants (see, e.g., Kitzman, et al. (2015) Nature Methods 12:203-206; Firnberg & Ostermeier (2012) PLOS One 7: e52031; Jain & Varadarajan (2014) Analytical Biochemistry 449:90-98; and Wrenbeck, et al. (2016) Nature Methods 13:928).

In particular embodiments, a deep mutational scanning library includes or encodes all possible amino acids at all positions of a protein, and each variant protein is encoded by more than one variant nucleotide sequence. In particular embodiments, a deep mutational scanning library includes or encodes all possible amino acids at all positions of a protein, and each variant protein is encoded by one nucleotide sequence.

In particular embodiments, a deep mutational scanning library includes or encodes all possible amino acids at less than all positions of a protein, for example at 10%, 20%, 30%, 40%, 50%, 60%, 70%, 80%, 90%, 95%, 96%, 97%, 98% or 99% of positions. In particular embodiments, a deep mutational scanning library includes or encodes less than all possible amino acids (for example 10%, 20%, 30%, 40%, 50%, 60%, 70%, 80%, 90%, 95%, 96%, 97%, 98% or 99% of potential amino acids) at all positions of a protein. In particular embodiments, a deep mutational scanning library includes or encodes less than all possible amino acids (for example 10%, 20%, 30%, 40%, 50%, 60%, 70%, 80%, 90%, 95%, 96%, 97%, 98% or 99% of potential amino acids) at less than all positions of a protein, for example at 10%, 20%, 30%, 40%, 50%, 60%, 70%, 80%, 90%, 95%, 96%, 97%, 98% or 99% of positions. A deep mutational scanning library can also include a set of variant nucleotide sequences that can collectively encode protein variants including at least a particular number of amino acid substitutions at at least a particular percentage of amino acid positions. “Collectively encode” takes into account all amino acid substitutions at all amino acid positions encoded by all the variant nucleotide sequences in total in a deep mutational scanning library. Libraries created using the methods described herein can also encode mutations at a pre-determined subset of sites within a protein of interest. References to mutational scanning libraries throughout the disclosure can include reference to deep mutational scanning libraries, historical libraries or libraries of currently circulating viruses can also be used.

In particular embodiments, a codon-mutant library can be generated by PCR, primer-based mutagenesis, as described in Example 1 and in US2016/0145603. Codon-mutant libraries can also be synthetically constructed by and obtained from a synthetic DNA company such as Twist Bioscience (San Francisco, CA). Methods to generate a codon-mutant library also include: nicking mutagenesis as described in Wrenbeck et al. Nature Methods 13:928-930 (2016) and Wrenbeck et al. Protocol Exchange doi: 10.1038/protex.2016.061 (2016); PFunkel (Firnberg & Ostermeier PLOS ONE 7 (12): e52031 (2012)); massively parallel single-amino-acid mutagenesis using microarray-programmed oligonucleotides (Kitzman et al. Nature Methods 12:203-206 (2015)); and saturation editing of genomic regions with CRISPR-Cas9 (Findlay et al. Nature 513 (7516): 120-123 (2014)).

Supporting the description of creating codon-mutant libraries, the following information is provided for viral entry proteins for influenza. Hemagglutinin (HA) is 566 codons long, so there are 566×63=35,658 codon mutations corresponding to 566×19=10,754 amino acid mutations. The number of mutations per clone from the mutagenesis method follows a Poisson distribution, and an average of 1.5 mutations can be introduced per clone and libraries of 5×10⁵ clones can be created. Therefore, 1.7×10⁵ of the clones will be single mutants, and 2.2×10⁵ will be multiple mutants. The typical single-codon mutant will thus be represented by 5 clones, and with Poisson statistics 99% of single-codon mutants should be captured in at least one clone. The typical single amino acid mutant will be represented by 15 clones, although this will vary among amino acids with different codon degeneracies. In particular embodiments, HA from A/Perth/16/2009 (H3N2), a recent component of the influenza vaccine can be used to generate a codon-mutant library with barcodes for HA. While hemagglutinin is provided as an example, similar calculations may be made for other viral entry proteins.

Each variant sequence can be associated with a barcode. In particular embodiments, the barcode is 18-nucleotides in length. Because there are 4¹⁸-7¹⁰ different 18-nucleotide sequences, virtually every variant can have a unique barcode. The barcode can be any appropriate length and composition that does not negatively affect fitness of the encoded variant protein. The barcode is a nucleotide sequence that allows identification of a variant within a library and distinction from other variants. It may be linked to a variant of interest by long read sequencing or Sanger sequencing and may be flanked on either end by constant sequences which may be used for priming and amplifying the barcode region for short read next generation sequencing analysis. In particular embodiments, the length of the barcode is based upon the size of the deep mutation scanning library. If more distinct barcodes are needed, then barcodes of greater length can be used. If less distinct barcodes are needed, then barcodes of lesser length can be used. In particular embodiments, the barcode can be 4-100 nucleotides in length, 5-100 nucleotides in length, 10-80 nucleotides in length, 10-50 nucleotides in length, 10-30 nucleotides in length, 8-30 nucleotides in length, 8 to 25 nucleotides in length, 12-24 nucleotides in length, or 16-20 nucleotides in length. In particular embodiments, the barcode can be 3 nucleotides in length, 4 nucleotides in length, 5 nucleotides in length, 6 nucleotides in length, 7 nucleotides in length, 8 nucleotides in length, 9 nucleotides in length, 10 nucleotides in length, 11 nucleotides in length, 12 nucleotides in length, 13 nucleotides in length, 14 nucleotides in length, 15 nucleotides in length, 16 nucleotides in length, 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, 29 nucleotides in length, 30 nucleotides in length, 31 nucleotides in length, 32 nucleotides in length, 33 nucleotides in length, 34 nucleotides in length, 35 nucleotides in length, 36 nucleotides in length, 37 nucleotides in length, 38 nucleotides in length, 39 nucleotides in length, 40 nucleotides in length, or more.

After creating barcoded influenza viruses or other barcoded viruses, each variant viral protein can be associated with its barcode. In particular embodiments, a high throughput sequencing method that can sequence long reads with high accuracy can be used to associate each viral protein variant with its barcode. For example, this can be conducted using circular consensus PacBio sequencing as described in Travers, et al. Nucleic Acids Research 38: e159-e159 (2010) and Laird Smith, et al. Virus Evolution 2: vew018 (2016). In particular embodiments, long reads can include greater than 100 bp, greater than 200 bp, greater than 300 bp, greater than 400 bp, greater than 500 bp, greater than 600 bp, greater than 700 bp, greater than 800 bp, greater than 900 bp, greater than 1000 bp, greater than 2000 bp, greater than 3000 bp, greater than 4000 bp, greater than 5000 bp, greater than 6000 bp, greater than 7000 bp, greater than 8000 bp, greater than 9000 bp, greater than 10,000 bp, or more. In particular embodiments, accuracy of a sequencing method is related to the sequencing method's error rate. A sequencing error rate can be expressed as a sequencing quality score of a given base, Q, defined by the following equation: Q=−10 log₁₀(e), where e is the estimated probability of the base call being wrong. Higher Q scores indicate a smaller probability of error. In particular embodiments, a Q score of 10 represents an error rate of 1 in 10 bases, and the inferred base call accuracy is 90%. A Q score of 20 can represent an error rate of 1 in 100 bases, and the inferred base call accuracy is 99%. A Q score of 30 can represent an error rate of 1 in 1000 bases, and the inferred base call accuracy is 99.9%. In particular embodiments, high accuracy includes having fewer systematic errors such as errors in base calling or read mapping/alignment and/or errors that are independent of the sequencing context. For example, a high throughput sequencing method that has errors independent of sequencing context would have the same error rate regardless if the sequence was AAAAAAAA (SEQ ID NO: 37) versus AAAAACAG (SEQ ID NO: 38). (DePristo et al. Nat Genet 43 (5): 491-498 (2011); Roberts et al. Genome Biology 14:405 (2013). In particular embodiments, high accuracy includes 99.99% accuracy.

In particular embodiments, each influenza virus variant can be associated with its barcode by subassembly as described in U.S. Pat. No. 8,383,345. It can also be associated with its barcode by long-read PacBio or Oxford Nanopore sequencing. In particular embodiments, if the gene encoding a variant influenza protein is small, each gene encoding the protein variant can be associated with its barcode by a barcoded subamplicon approach as described above and in Doud & Bloom Viruses 8, 155 (2016).

(iii) Exposure to Selection Pressures. Following creation of a barcoded influenza virus mutational scanning library, members of the library can be exposed to a selection pressure to assess the variant virus' resistance or susceptibility to the selection pressure. In some aspects, a plurality of section pressures may be applied. Further, the cumulative impact of mutations can be assessed by comparing data from sampled variants from the pdmH1N1 lineage to a combinatorial library, and the cumulative impact of mutations can be assessed, including identifying if recent mutations take advantage of pre-existing holes in immunity even if they naturally occurred in variants that were already antigenically distinct.

The naturally occurring mutation rate of the influenza A virus RdRP is in the range of 2.0×10⁻⁶ to 2.0×10⁻⁴ mutations per site per round of genome replication (Parvin, J. D., Moscona, A., Pan, W. T., Leider, J. M. & Palese, P. Measurement of the mutation rates of animal viruses: influenza A virus and poliovirus type 1. J. Virol. 59, 377-383 (1986)). Thus, the probability of generating a specific antigenic drift variant through a single nucleotide mutation is 2/10⁵ (Pauly, M. D., Procario, M. C. & Lauring, A. S. A novel twelve class fluctuation test reveals higher than expected mutation rates for influenza A viruses. eLife 6, e26437 (2017)). The rate of mutation can be increased through the use of selection pressure.

In particular embodiment, the selection pressure impacts the ability of the virus to enter (i) a host cell of a target host species or (ii) a cell expressing a receptor protein of a species that is different from the species from which the cell was derived, wherein the ability is not dependent on presence of a functional unrelated viral entry protein. The target host cell may be any mammalian species, for example, human, pig, bat, or camel, as well as avian species such as poultry or waterfowl. In some aspects, the bat cell lines are derived from fruit bat lung, fruit bat kidney, Egyptian fruit bat, or pipestrelle bat. In particular embodiments, the target host species are from human cell lines such as human liver, human lung, or human lung epithelia. In particular embodiments, the human cell line derived from human liver includes HuH7, the human cell line derived from human lung includes Calu-3 or MRC-5, and/or the human cell line derived from human lung epithelia is A549 or BEAS-2B.

A selection pressure can include one or more environmental conditions that may affect a virus's function or survival. For example, the environmental condition may include exposure to a therapeutic compound or to heat. Selection pressure may also be caused by an immune response in a host organism. Numerous selection pressures are described in additional detail in this section.

In particular embodiments, the selection pressure is exposure a putative neutralizing agent such as a compound that may have therapeutic efficacy against influenza infection or other virus of concern. In particular embodiments, the compound is one that is described in, for example, U.S. Pat. Nos. 5,994,515, 9,259,433, US2009/0214510, US2017/0157190, WO2008/147427, WO2009/027057, WO2009/151313, WO2012/006596, WO2013/006795, WO2013/072917, and WO2014/062892; Laursen and Wilson (2013) Antiviral Res 98(3): 476-483; and Pelegrin et al. (2015) Trends in Microbiology 23 (10): 653-665.

In particular embodiments, compounds for assessment can include putative neutralizing agents such as anti-virals such as anti-influenza virus antibodies including TNX-355 (ibalizumab); PGT121 (Julien et al. (2013) PLOS Pathog 9(5): e1003342; broadly neutralizing antibody); and 3BNC117 (Scheid et al. (2016) Nature. 535:556-560). Other anti-viral antibodies may be used for other viruses.

In particular embodiments, compounds can include viral entry and/or fusion inhibitors. Entry and fusion inhibitors can include, for example, highly sulfated polysaccharides from fucoidan or algae; calcium spirulan, nostoflan, or extract of Scoparia dulcis, or antiviral diterpene components contained therein, such as scoparic acid A, scoparic acid B, scoparic acid C, scopodiol, scopadulin, scopadulcic acid A (SDA), scopadulcic acid B (SDB), and/or scopadulcic acid C (SDC).

In particular embodiments, compounds can include influenza virus polymerase inhibitors, drugs that increase the viral mutation rate, drugs that interfere with function of the hemagglutinin or neuraminidase protein, and inhibitors that inhibit binding of an influenza virus genome to one or more nucleoproteins. In particular embodiments, compounds are directly or indirectly effective in specifically interfering with at least one virus action including penetration of eukaryotic cells, replication in eukaryotic cells, virus assembly, release from infected eukaryotic cells, or that is effective in nonspecifically inhibiting a virus titer increase or in nonspecifically reducing a virus titer level in a eukaryotic or mammalian host system.

In particular embodiments, the selection pressure is a toxic agent. Toxic agents can include polar organic solvents (e.g., dimethylformamide), herbicides (e.g., glyphosate), pesticides (e.g., malathion, dichlorodiphenyltrichloroethane), salinity, ionizing radiation, and hormonally active phytochemicals (e.g., flavonoids, lignins and lignans, coumestans, or saponins).

In particular embodiments, mutational scanning libraries described herein can be used to perform virus resistance analysis to putative neutralizing agents such as therapeutic compounds including therapeutic compounds undergoing clinical and pre-clinical trials. In some aspects, the putative neutralizing agent including a therapeutic compound may include a small molecule, a protein, a peptide, a polynucleotide, a polysaccharide, an oil a solution, or a plant extract. In these embodiments, virus resistance to therapeutic compounds caused by mutations of given protein residues represented within the mutational scanning can be assessed.

In particular embodiments, in vitro resistance analysis studies can assess the potential ability of a virus to develop resistance to a therapeutic compound and to help in designing clinical studies. Virus resistance to a given therapeutic compound can be selected in cell culture, and the selection can provide a genetic threshold for resistance development. For example, a therapeutic compound with a low genetic threshold may become susceptible to viral resistance with only one or two mutations. In contrast, a therapeutic compound with a high genetic threshold may require multiple mutations to become susceptible to viral resistance. Therapeutic compounds with higher genetic thresholds can be selected for further clinical development.

In particular embodiments, the development of viral resistance in vitro can be assessed over a concentration range of a therapeutic compound spanning the anticipated concentration of the therapeutic compound that will be used in vivo. Selection of variants resistant to a therapeutic compound can be repeated more than once (e.g., with different strains of wild-type, with resistant strains, under high and low selective pressures) to determine if the same or different patterns of resistance mutations develop, and to assess the relationship of therapeutic compound concentration to the resistance.

As discussed above, determining the mutations that might contribute to reduced susceptibility to a therapeutic compound using the systems and methods of the present disclosure can include sequencing barcodes after linking a barcode to a particular viral protein variant in a mutational scanning library. Identifying resistance mutations by this genotypic analysis can be useful in predicting clinical outcomes and supporting the proposed mechanism of action of a therapeutic compound. In particular embodiments, the pattern of mutations leading to resistance of a therapeutic compound can be compared with the pattern of mutations of other therapeutic compounds in the same class. In particular embodiments, resistance pathways can be characterized in several genetic backgrounds (i.e., strains, subtypes, genotypes) and protein variants can be obtained throughout the selection process to identify the order in which multiple mutations appear.

Phenotypic analysis determines if mutant viruses have reduced susceptibility to a therapeutic compound. In particular embodiments, using the systems and methods of the present disclosure, phenotypic analysis is performed when influenza virions including protein variants are selected for resistance to a therapeutic compound. In particular embodiments, phenotypic resistance can be scored, for example, by an EC₅₀ value. An EC₅₀ value can refer to an effective concentration of a therapeutic compound which induces a response halfway between the baseline and maximum after a specified exposure time. In particular embodiments, an EC₅₀ value can be used as a measure of a therapeutic compound's potency. EC₅₀ can be expressed in molar units (M), where 1 M is equivalent to 1 mol/L. The fold resistant change can be calculated as the EC₅₀ value of the variant protein/EC₅₀ value of a reference protein. Phenotypic results can be determined with any standard virus assay (e.g., protein assay, viral RNA assay, polymerase assay, MTT cytotoxic assay, reporter or selectable marker expression). In particular embodiments, influenza virus titer can be calculated as a function of the concentration of the therapeutic compound to obtain an EC₅₀ value. In particular embodiments, influenza virus titer can be calculated by a plaque assay or focus forming assay. A plaque assay takes advantage of plaques that can arise through influenza virus-mediated cell death within a monolayer of a cell culture when cells are infected with an influenza virus and typically requires plaques to grow until visible to the naked eye. The focus-forming assay can be used to titer non-cytopathic influenza viruses. This assay usually relies on the detection of infected cells by immunostaining for influenza virus antigen or via a genetically encoded fluorescent reporter. The shift in susceptibility (or fold resistant change) for a protein variant can be measured by determining the EC₅₀ value for the variant protein and comparing it to the EC₅₀ value of a reference protein. In particular embodiments, a reference protein can be a counterpart influenza viral protein (equivalent viral protein having the same function from the same viral strain) from a wild-type virus, from a well-characterized wild-type laboratory strain, from a parental virus, or from a baseline clinical isolate done under the same conditions and at the same time. In particular embodiments, a wild-type virus can be naturally occurring. In particular embodiments, a wild-type virus has no mutations that confer drug resistance. In particular embodiments, a parental virus can be an influenza virus having a viral protein that did not undergo mutagenesis as described herein to create a barcoded mutational scanning library of variants of the influenza viral protein. In particular embodiments, a parental virus can be a wild type virus. A baseline clinical isolate includes an isolate from a subject being screened for inclusion in a clinical trial or an isolate from a subject in a clinical trial before treatment in the trial has begun. The use of the EC₅₀ value for determining shifts in susceptibility can offer greater precision than an EC₉₀ or EC₉₅ value. The utility of a phenotypic assay depends on its sensitivity (i.e., its ability to measure shifts in susceptibility (fold resistance change) in comparison to a reference). Calculating the fold resistant change (EC₅₀ value of variant protein/EC₅₀ value of reference protein) allows for comparisons among phenotypic assays.

A viral protein may develop mutations that lead to reduced susceptibility (i.e., resistance) to one antiviral therapeutic compound and can result in decreased or loss of susceptibility to other antiviral therapeutic compounds in the same therapeutic compound class. This observation is referred to as cross-resistance. Cross-resistance is not necessarily reciprocal, so it is important to evaluate both possibilities. For example, if influenza virus X is resistant to drug A and drug B, and influenza virus Y is also resistant to drug A, influenza virus Y may still be sensitive to drug B. In particular embodiments, the effectiveness of a therapeutic compound against viruses resistant to other approved therapeutic compounds in the same class and the effectiveness of approved therapeutic compounds belonging to a given class against influenza viruses resistant to a therapeutic compound belonging to that same class can be evaluated by phenotypic analyses. In particular embodiments, cross-resistance can be analyzed between therapeutic classes in instances where more than one therapeutic compound class targets a single influenza virus protein or protein complex (e.g., neuraminidase inhibitor and polymerase inhibitor, such as oseltamivir and baloxivr). Variant influenza virus proteins representative of the breadth of diverse mutations and combinations of mutations known to confer reduced susceptibility to therapeutic compounds in the same class can be tested for phenotypic susceptibility to a new therapeutic compound belonging to that same class.

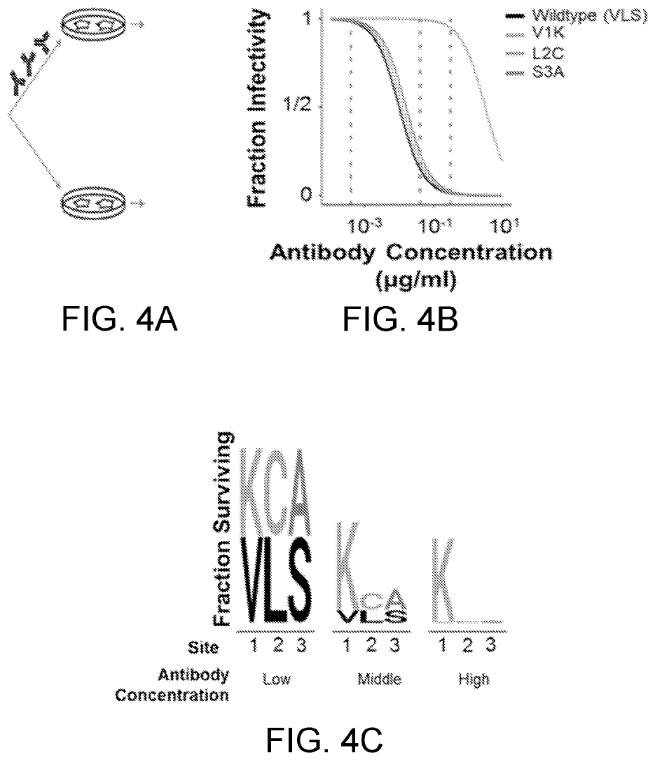

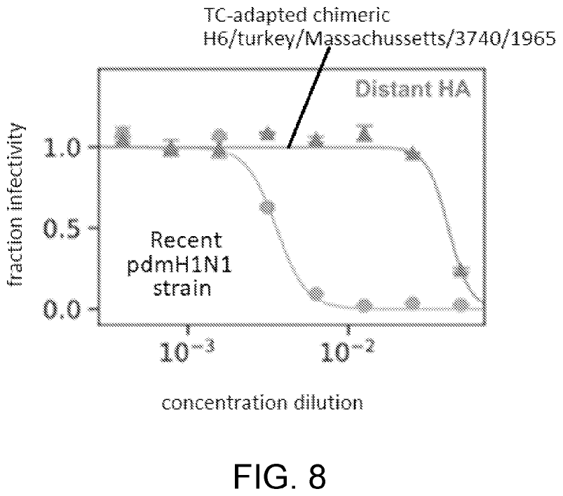

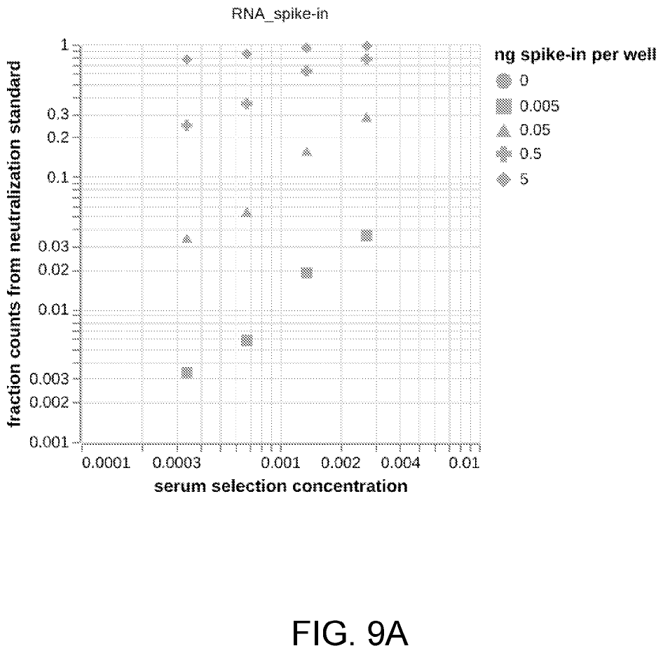

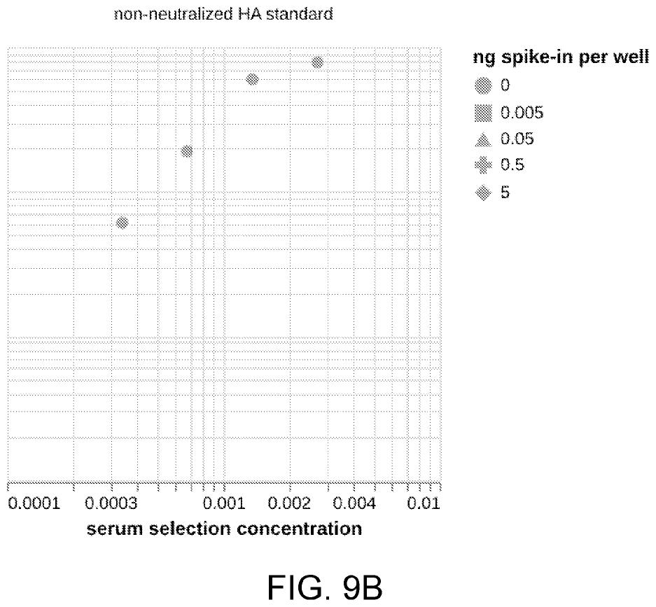

The sensitivity of a virus to an antibody or serum sample can be quantified by a neutralization curve (FIG. 4B). Such curves are conventionally measured on individual viral variants, but they can in principle be measured for many variants at once using deep sequencing. In some aspects, a control virus may be used as a comparison. For example, to obtain neutralization curves in particular embodiments, the absolute fraction of each influenza virus variant that survives exposure to an antibody or sera or other putative neutralizing agent can be measured by combining non-neutralized HA virus with a virus library and then incubated with serially diluted human serum, Following incubation, the virus-serum mix may be added to cells. After infection, viral RNA may be extracted from the cells and samples mixed with a constant amount of RNA spike-in standard allowing for the identification of barcode counts that correspond to either variants within the library or the non-neutralized viral standard as shown in FIGS. 9A and 9B. Data may be transformed into percent reads corresponding to non-neutralized standard as shown in FIG. 8. For an absolute standard, virions with surface proteins from a non-human influenza virus subtype can be used, such as subtypes H4, H6, or H14. Such, distantly related, functional influenza hemagglutinins (HAs) may be used as internal standards for experiments with barcoded influenza. For example, the proteins of subtypes H6 and H8 as shown in SEQ ID NO:39-42 may be used as internal standards. Most humans have limited neutralization activity against these distant HAs. Including a distant HA as an internal standard for non-neutralized virus growth allows for quantitative measurement of the impact of mutations on neutralization.

In particular embodiments, any viral surface protein not affected by the antibody or sera can be used as an absolute standard. With these standards, neutralization curves can be generated by incubating the virus libraries at several antibody concentrations, infecting cells with the treated viruses, and sequencing the barcodes. The fraction of each mutant surviving relative to the standards can be computed. In particular embodiments, the use of two standards will allow detection of whether one is unexpectedly affected by the antibody. Neutralization curves can be fit and the data can be represented as in FIG. 4B.

In prior work, deep sequencing of viral libraries was used to measure antibody selection on viral mutations (Doud, et al. PLOS Pathog. 13 (3): e1006271 (2017); Dingens et al., Cell Host & Microbe 21:777-787 (2017); Doud et al. bioRxiv DOI: 210468 (2018)). Because these libraries were not barcoded, it was only feasible to use one or a few antibody concentrations. With the barcoded libraries disclosed herein, multiple concentrations to interpolate full curves can be tested. In particular embodiments, curves for >10⁴ mutants can be generated. In these embodiments, it can be more informative to represent the results in logo plots rather than overlaying vast numbers of curves (FIG. 4C). In particular embodiments, a sequence logo plot can be a graphical representation of sequence conservation of nucleotides or amino acids. A sequence logo can be created from a collection of aligned sequences and depicts the consensus sequence and diversity of the sequences. In particular embodiments, sequence logos can be used to depict sequence characteristics such as protein-binding sites in DNA or functional units in proteins. In particular embodiments, sequence logos can be used to depict the preference for a nucleotide base or an amino acid residue at a given position in a nucleotide sequence or in an amino acid sequence, respectively. In particular embodiments, sequence logos can be used to depict the effect of each amino acid or nucleotide on a selective pressure, such as antibody neutralization or drug inhibition as described above.

In particular embodiments, the selection pressure is heat. Heat can include temperatures above 25° C., above 26° C., above 27° C., above 28° C., above 29° C., above 30° C., above 31° C., above 32° C., above 33° C., above 34° C., above 35° C., above 36° C., above 37° C., above 38° C., above 39° C., above 40° C., above 41° C., above 42° C., above 43° C., above 44° C., above 45° C., above 46° C., above 48° C., above 49° C., above 49° C., above 50° C., or more. In particular embodiments, heat can include temperatures from 28° C. to 70° C. In particular embodiments, heat can include temperatures from 30° C. to 65° C. In particular embodiments, heat can include temperatures above 30° C. In particular embodiments, the selection pressure is cold. Cold can include temperatures below 25° C., below 24° C., below 23° C., below 22° C., below 21° C., below 20° C., below 19° C., below 18° C., below 17° C., below 16° C., below 15° C., below 14° C., below 13° C., below 12° C., below 11° C., below 10° C., below 9° C., below 8° C., below 7° C., below 6° C., below 5° C., below 4° C., below 3° C., below 2° C., below 1° C., below 0° C., or lower. In particular embodiments, cold can include temperatures from 22° C. to 0° C. In particular embodiments, cold can include temperatures from 20° C. to 4° C. In particular embodiments, cold can include temperatures below 20° C. In particular embodiments, the selection pressure is low pH. Low pH can include pH of 6.9, 6.5, 6.0, 5.5, 5.0, 4.5, 4.0, 3.5, 3.0, 2.5, 2.0, or lower. In particular embodiments, low pH can be from pH of 6.8 to 2.0. In particular embodiments, low pH can be from pH of 6.5 to 3.0. In particular embodiments, low pH can include a pH below 6.5. In particular embodiments, the selection pressure is high pH. High pH can include pH of 7.5, 7.6, 7.7, 7.8, 7.9, 8.0, 8.5, 9.0, 9.5, 10.0, 10.5, 11.0, 11.5, 12.0, or higher. In particular embodiments, high pH can include pH of 8.0 to 14.0. In particular embodiments, high pH can include pH of 8.5 to 12.0. In particular embodiments, high pH can include a pH above 8.0.

(iv) Engineering More Effective Antibodies. The systems and methods of the present disclosure can be used to engineer antibodies that are more effective in neutralizing a viral protein. In particular embodiments, a method of engineering a second, more effective therapeutic antibody from a first antibody against a virus using a barcoded influenza virus mutational scanning library can include: obtaining the barcoded influenza virus library wherein the barcoded influenza virus variants collectively provide viral protein variants including at least 15 amino acid substitutions at at least 95% of amino acid positions of the viral protein under analysis; exposing target cells to (i) the virions and (ii) the first antibody; sequencing barcodes following exposure to the first antibody, wherein the barcodes associated with variant nucleotide sequences conferring an ability to evade the first antibody increase in frequency and the barcodes associated with variant nucleotide sequences conferring an inability to evade the first antibody decrease in frequency; comparing variant nucleotide sequences conferring an ability to evade the first antibody with the nucleotide sequence of a reference viral protein that the first antibody binds; modifying amino acid residues in the first antibody based on the comparing and on a known crystal structure of the reference viral protein/first antibody complex, thereby engineering a second, more effective therapeutic antibody from a first antibody against the virus. In particular embodiments, engineering a more effective antibody can include the method described in Diskin et al. (2013) J. Exp. Med. 210(6): 1235-1249.

Naturally occurring antibody structural units include a tetramer. Each tetramer includes two pairs of polypeptide chains, each pair having one light chain and one heavy chain. The amino-terminal portion of each chain includes a variable region that is responsible for antigen recognition and epitope binding. The variable regions exhibit the same general structure of relatively conserved framework regions (FR) joined by three hyper variable regions, also called complementarity determining regions (CDRs). The CDRs from the two chains of each pair are aligned by the framework regions, which enables binding to a specific epitope. From N-terminal to C-terminal, both light and heavy chain variable regions include the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md. (1987 and 1991)), or Chothia & Lesk, J. Mol. Biol., 196:901-917 (1987); Chothia et al., Nature, 342:878-883 (1989).

The carboxy-terminal portion of each chain defines a constant region that can be responsible for effector function. Examples of effector functions include: C1q binding and complement dependent cytotoxicity (CDC); antibody-dependent cell-mediated cytotoxicity (ADCC); antibody-dependent phagocytosis (ADCP); down regulation of cell surface receptors (e.g., B cell receptors); and B cell activation.

Within full-length light and heavy chains, the variable and constant regions are joined by a “J” region of amino acids, with the heavy chain also including a “D” region of amino acids. See, e.g., Fundamental Immunology, Ch. 7 (Paul, W., ed., 2nd ed. Raven Press, N.Y. (1989).

Unless otherwise indicated, the term “antibody” includes, in addition to antibodies including two full-length heavy chains and two full-length light chains as described above, variants, derivatives, and fragments thereof, examples of which are described below. Furthermore, unless explicitly excluded, antibodies can include monoclonal antibodies, human antibodies, bispecific antibodies, polyclonal antibodies, linear antibodies, minibodies, domain antibodies, synthetic antibodies, chimeric antibodies, antibody fusions, and fragments thereof, respectively. In particular embodiments, antibodies (e.g., full length antibodies) can be produced in human suspension cells.

In particular embodiments, monoclonal antibodies refer to antibodies produced by a clone of B cells or hybridoma cells. In particular embodiments, monoclonal antibodies are identical to each other and/or bind the same epitope, except for possible antibodies containing naturally occurring mutations or mutations arising during production of a monoclonal antibody. In particular embodiments, in contrast to polyclonal antibody preparations, which include different antibodies directed against different epitopes, each monoclonal antibody of a monoclonal antibody preparation is directed against a single epitope on an antigen.

A “human antibody” is one which includes an amino acid sequence which corresponds to that of an antibody produced by a human or a human cell or derived from a non-human source that utilizes human antibody repertoires or other human antibody-encoding sequences.

A “human consensus framework” is a framework which represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. The subgroup of sequences can be a subgroup as in Kabat et al., Sequences of Proteins of Immunological Interest, Fifth Edition, NIH Publication 91-3242, Bethesda Md. (1991), vols. 1-3. In particular embodiments, for the V_{L}, the subgroup is subgroup kappa I as in Kabat et al., supra. In particular embodiments, for the V_{H}, the subgroup is subgroup III as in Kabat et al., supra.

In particular embodiments, an antibody fragment is used. An “antibody fragment” denotes a portion of a complete or full-length antibody that retains the ability to bind to an epitope. Examples of antibody fragments include Fv, single chain Fv fragments (scFvs), Fab, Fab′, Fab′-SH, F(ab′)₂, diabodies, linear antibodies, and/or any biologically effective fragments of an immunoglobulin that bind specifically to an epitope described herein. Antibodies or antibody fragments include all or a portion of polyclonal antibodies, monoclonal antibodies, human antibodies, humanized antibodies, synthetic antibodies, chimeric antibodies, bispecific antibodies, mini bodies, and linear antibodies.

A single chain variable fragment (scFv) is a fusion protein of the variable regions of the heavy and light chains of immunoglobulins connected with a short linker peptide. Fv fragments include the VL and VH domains of a single arm of an antibody. Although the two domains of the Fv fragment, VL and VH, are coded by separate genes, they can be joined, using, for example, recombinant methods, by a synthetic linker that enables them to be made as a single protein chain in which the VL and VH regions pair to form monovalent molecules (single chain Fv (scFv)). For additional information regarding Fv and scFv, see e.g., Bird, et al., Science 242 (1988) 423-426; Huston, et al., Proc. Natl. Acad. Sci. USA 85 (1988) 5879-5883; Plueckthun, in The Pharmacology of Monoclonal Antibodies, vol. 113, Rosenburg and Moore (eds.), Springer-Verlag, New York), (1994) 269-315; WO1993/16185; U.S. Pat. Nos. 5,571,894 and 5,587,458.

A Fab fragment is a monovalent antibody fragment including V_{L}, V_{H}, CL and C_{H1} domains. A F(ab′)₂ fragment is a bivalent fragment including two Fab fragments linked by a disulfide bridge at the hinge region. For discussion of Fab and F(ab′)₂ fragments having increased in vivo half-life, see U.S. Pat. No. 5,869,046. Diabodies include two epitope-binding sites that may be bivalent. See, for example, EP 0404097; WO1993/01161; and Holliger, et al., Proc. Natl. Acad. Sci. USA 90 (1993) 6444-6448. Dual affinity retargeting antibodies (DART™; based on the diabody format but featuring a C-terminal disulfide bridge for additional stabilization (Moore et al., Blood 117, 4542-51 (2011)) can also be used. Antibody fragments can also include isolated CDRs. For a review of antibody fragments, see Hudson, et al., Nat. Med. 9 (2003) 129-134.

Antibody fragments can be made by various techniques, including proteolytic digestion of an intact antibody as well as production by recombinant host-cells (e.g., human suspension cell lines, E. coli or phage), as described herein. Antibody fragments can be screened for their binding properties in the same manner as intact antibodies.

A neutralizing antibody can refer to an antibody that, upon epitope binding, can reduce biological function of its target antigen. In particular embodiments neutralizing antibodies can reduce (i.e., neutralize) viral infection of cells. In particular embodiments percent neutralization can refer to a percent decrease in viral infectivity in the presence of the antibody, as compared to viral infectivity in the absence of the antibody. For example, if half as many cells in a sample become infected in the presence of an antibody, as compared to in the absence of the antibody, this can be calculated as 50% neutralization. In particular embodiments “neutralize viral infection” can refer to at least 40% neutralization, at least 50% neutralization, at least 60% neutralization, at least 70% neutralization, at least 80% neutralization, or at least 90% neutralization of viral infection. In particular embodiments, the antibodies can block viral infection (i.e., 100% neutralization). In particular embodiments, the anti-viral antibodies can inhibit envelope fusion with target cells, which can result in neutralization of viral infection. Inhibition of viral envelope fusion to target cells can be at least 40% inhibition, at least 50% inhibition, at least 60% inhibition, at least 70% inhibition, at least 80% inhibition, or at least 90% inhibition, as compared to viral envelope fusion in the absence of the anti-viral antibody.

In particular embodiments, an antibody that neutralizes a viral infection is effective against the virus.

(v) Vaccine Development. As one example, in particular embodiments, sera samples can be obtained from vaccine studies to map mutations that affect resistance to these sera. This work can functionally map the epitopes targeted by the vaccines and enable correlation of animal-to-animal variation in protection with variation in epitope targeting, both of which could help inform further immunogen design. Immunogen may also be influenced by deep mutational scanning of libraries to identify mutations that would reduce viral virulence but still trigger an immune response. Deep mutational scanning may also be used to identify proteins that may be used to create virus-like particles. With the NGS-based neutralization assay, neutralization potency against all variants included in the library can be obtained with a single dilution series, allowing for rapid testing of candidates. This work can improve forecasting of viral evolution and guide the development of vaccines and antivirals.

(vi) Selection of Effective Anti-Viral Conditions and/or Effective Therapeutic Compounds. Assessments described herein can be used to select effective anti-viral conditions and/or effective therapeutic compounds.

An effective therapeutic compound refers to a compound that can reduce, prevent, or treat influenza virus infection when the compound is administered to a subject. In particular embodiments, an effective therapeutic compound can prevent, reduce, or treat the likelihood of an influenza virus infection.

An amount of the therapeutic compound that is effective will vary depending on the compound, the severity or risk of infection, and the age, weight, physical condition and responsiveness of the subject to be treated. The exact dose and formulation will depend on the purpose of the treatment and can be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)).

In certain cases, a “therapeutically effective amount” is used to mean an amount or dose sufficient to modulate, e.g., increase or decrease a desired activity e.g., by 10%, by 50%, or by 90%. Generally, a therapeutically effective amount is sufficient to cause a clinically significant improvement in a subject following a therapeutic regimen involving one or more therapeutic compounds. The concentration or amount of the compound depends on the desired dosage and administration regimen. The effective amounts of compounds containing active agents include doses that partially or completely achieve the desired therapeutic, prophylactic, and/or biological effect.

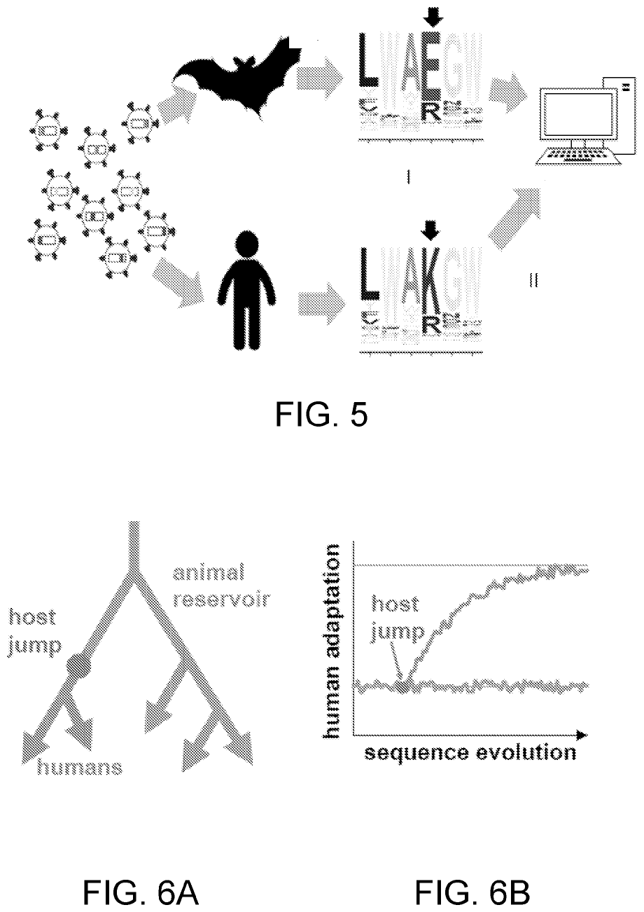

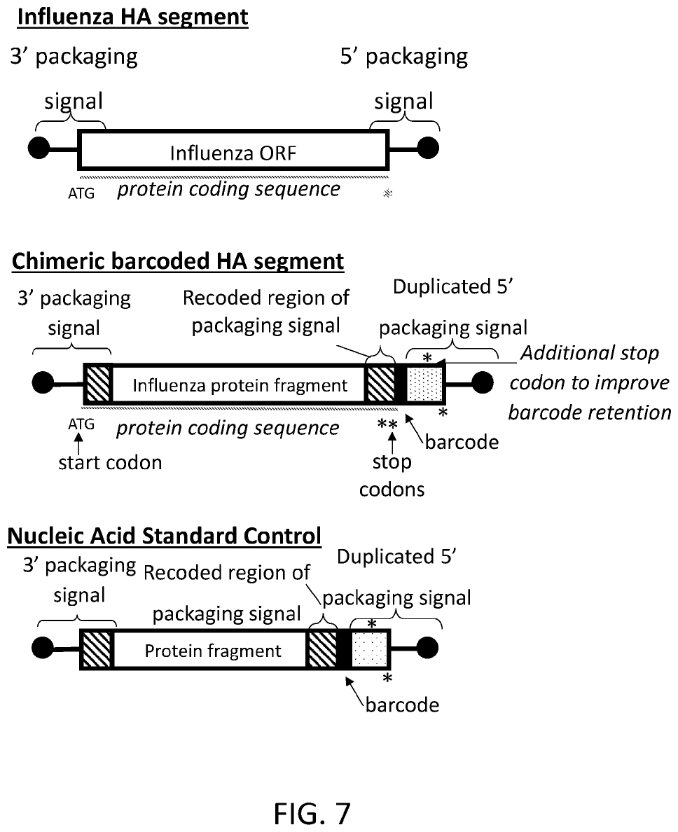

(vi) Host Adaptation Studies. To enable identification of host adaptation, how mutations affect each viral entry protein's ability to mediate infection of cells from relevant host species can be measured (FIGS. 5, 6A, 6B). In particular embodiments, methods described herein measure the preference for each amino acid at each site in a viral entry protein under selection to infect different cell lines.

Using an HA library as an example, the libraries can be used to measure the functional effects of all mutations to HA. Viral infectivity will depend on HA. The virions can be used to infect cells (e.g., MDCK-SIAT1 cells). Then, viral RNA can be isolated and the barcodes can be sequenced to quantify the variant frequencies in each case. Since the typical single amino acid mutant will have 15 barcodes, this gives >100 counts for the typical mutation in the unselected condition. Counts in the selected condition will vary depending on the functionality of that particular HA mutant. Algorithms to extract functional information from mutational scanning counts have been described and implemented. These algorithms can be used to estimate the “preference” of each site in HA for each amino acid (see FIG. 5). Such preferences are a useful way to represent the data since they can be related to viral evolution in nature using phylogenetic methods (Hilton, et al. PeerJ 5: e3657 (2017)). In particular embodiments, the preferences can be estimated using barcode counts for single amino acid mutants. Preferences for multiple mutations can also be estimated. Other alternative strategies for estimating the effects of mutations from the sequencing data can also be used.

As exemplary uses, the libraries can be used to map how all mutations to entry proteins of influenza virus strains affect capacity to infect cells from relevant species. Certain influenza virus strains circulate in animal reservoirs but occasionally transmit to humans. These viruses could therefore cause epidemics or pandemics if they adapt to better infect and transmit among humans.

Differences that are host-specific rather than cell-line specific can often be more interesting. Accordingly, in particular embodiments, multiple cell lines for all hosts can be used to identify mutations that are robustly favored in numerous or all cell lines of that host.

In particular embodiments, (i) duplicate libraries, (ii) the existence of a few barcodes to hundreds of barcodes for each amino acid mutant, and (iii) algorithms similar to those in Haddox et al. eLife 7: e34420 (2018)) can be used to quantify noise and identify cell-line-specific differences that exceed this noise.

Results across more than one strain of a virus can be used to determine the extent that mutations are generally host adaptive versus strain-specific effects because viral strains can be genetically diverse (see Haddox et al. eLife 7: e34420 (2018)). Using, for example, two or more strains of a virus allows assessment of how well the measurements can be generalized across strains. In particular embodiments, assessing strain-specificity can be important in order to use the methods to better score host adaptation. Another way to examine this question is via the multiple mutants in the libraries. Particularly, whether effects of multiple mutations are the sum of the effects of the individual mutations can be assessed under an optimal scale as determined in Sailer et al. Genetics 205:1079-1088 (2017).

As indicated, in particular embodiments, measurements can be used to develop algorithms that score a virus's host adaptation from its sequence. This will advance assessment of the risk of viral host jumps (Russell et al. eLife 3: e03883 (2014)), and improve the ability to identify viral adaptation during human outbreaks.

In particular embodiments, host scoring can be performed using an additive model. For example, if

$${π}_{r , a}^{h}$$

is the preference for amino acid a at site r measured in cells from host h (e.g., the logo plots in FIG. 5), then the adaptation to host h of sequence s is scored as

$${S}_{h} ( s ) = {\sum }_{r} log ( {π}_{r , {s}_{r}}^{h} )$$

where sᵣ is the amino acid at site r of sequence s.

Historical data can be used to evaluate the scoring models. While additive models might seem simplistic, similar models informed by mutational scanning discriminated the evolutionary success of human influenza virus lineages (Lee, et al. Proceedings of the National Academy of Sciences, 115(35), E8276-E8285 (2018)), which is probably a harder problem since fitness differences between human influenza variants are likely smaller than those between variants of emerging viruses that have and have not adapted to humans.

As measurements for multiple mutations and different strain backgrounds are accumulated, epistatic models that incorporate non-additivity in forms can be explored (see, e.g., Louie et al. Proceedings of the National Academy of Sciences: 201717765 (2018); Hopf et al. Nature Biotechnology 35:128 (2017); Poelwijk et al. Learning the pattern of epistasis linking genotype and phenotype in a protein. bioRxiv: 213835 (2017); Sailer & Harms PLOS Computational Biology 13: e1005541 (2017)).

In particular embodiments, the systems and methods disclosed herein can be used to assess whether antigenic selection drives viral evolution. For example, it is unclear if immune selection drives the evolution of emerging virus strains. Uses of the libraries disclosed herein can identify sites where mutations affect immune recognition. Whether these immune-targeted sites evolve faster than other sites can be assessed. For example, one can fit codon-substitution models where the relative rate of amino acid substitution (dN/dS) is uniform across the gene or takes on a different value at sites experiments map as being under immune selection. HyPhy (Pond & Muse (2005) HyPhy: hypothesis testing using phylogenies. In: Statistical Methods in Molecular Evolution, Springer. pp. 125-181) can be used to fit these models, and a likelihood-ratio test to evaluate the support for the partitioned model versus the nested non-partitioned alternative can be used. Issues associated with strain specificity can also apply in these uses. That is, it may be that the antigenic effects of mutations vary among the strains of a virus. However, this issue can be assessed. These uses are based on the idea that epitopes are similar among different sera, but different sera could target very different epitopes due to host-to-host variation. In that case the generality of the mapping is reduced, but the throughput of disclosed methods then provides a way to characterize this variation, which is interesting in its own right.

(viii) Kits. Combinations of elements of the mutational scanning libraries disclosed herein can be provided as kits. Kits of the present disclosure can include: expression plasmids expressing barcoded influenza virus; one or more cell lines; transfection reagents; and a reference viral protein. In particular embodiments, the plasmids can be ambisense to allow both transcription of negative sense vRNA and expression of the viral protein encoded by the coding region of the vRNA. In particular embodiments, the reference viral protein is not recognized by sera that recognizes a viral protein in the barcoded influenza virus. In particular embodiments kits can include a mutational scanning library of barcoded influenza virus as disclosed herein. In particular embodiments, kits can include reagents for creating a deep mutation scanning library of barcoded influenza virus in expression plasmids such as reverse transcriptase, polymerase, amplification reagents (e.g., dNTPs, buffers, salts, etc.), packaging signal sequences, primers without barcodes, primers with barcodes, ligase, and restriction enzymes for generating expression plasmids including barcoded influenza genome segments with one or more inserted copy of a packaging signal.

Kits can include further instructions for using the kit, for example, instructions for transfection of cell lines expression plasmids expressing barcoded with transcription of negative sense vRNA and/or for expression of viral proteins from plasmids. The instructions can be in the form of printed instructions provided within the kit or the instructions can be printed on a portion of the kit itself. Instructions may be in the form of a sheet, pamphlet, brochure, CD-Rom, or computer-readable device, or can provide directions to instructions at a remote location, such as a website. In particular embodiments, kits can also include laboratory supplies needed to use the kit effectively, such as culture media, buffers, enzymes, sterile plates, sterile flasks, pipettes, gloves, and the like. Variations in contents of any of the kits described herein can be made.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (IX) EXEMPLARY EMBODIMENTS

1. A method for barcoding an influenza virus genome segment including: inserting a nucleic acid barcode and a copy of a coding region of a 5′ viral RNA genome packaging signal between a terminus of a corresponding genome segment open reading frame and a naturally occurring non-coding portion of the 5′ viral RNA genome packaging signal; and inserting at least one stop codon in the influenza virus genome segment;
   wherein the copy of the coding region of the 5′ viral RNA genome packaging signal has 40% to 75% sequence identity with a naturally occurring 5′ viral RNA genome packaging signal.
2. The method of embodiment 1, wherein the copy of the coding region of the 5′ viral RNA genome packaging signal has about 45% to 65% sequence identity with the naturally occurring 5′ viral RNA genome packaging signal.
3. The method of embodiment 1 or 2, wherein the copy of the coding region of the 5′ viral RNA genome packaging signal has about 40% to 50% sequence identity with the naturally occurring 5′ viral RNA genome packaging signal.
4. The method of any of embodiments 1-3, wherein the copy of the coding region of the 5′ viral RNA genome packaging signal has about 60% to 70% sequence identity with the naturally occurring 5′ viral RNA genome packaging signal.
5. The method of any of embodiments 1-4, wherein the copy of the coding region of the 5′ viral RNA genome packaging signal has 48% sequence identity with the naturally occurring 5′ viral RNA genome packaging signal.
6. The method of any of embodiments 1-5, wherein the coding region of the 5′ viral RNA genome packaging signal has 62% sequence identity with the naturally occurring 5′ viral RNA genome packaging signal.
7. The method of any of embodiments 1-6, wherein the at least one stop codon is inserted after a stop codon for the open reading frame within the 5′ viral RNA genome packaging signal that occurs after the barcode.
8. The method of any of embodiments 1-7, including inserting a plurality of stop codons after a stop codon for the open reading frame within the 5′ viral RNA genome packaging signal that occurs after the barcode.
9. The method of embodiment 8, wherein the plurality of stop codons after a stop codon for the open reading frame within the 5′ viral RNA genome packaging signal that occurs after the barcode are noncontiguous.
10. The method of method of any of embodiments 1-9, wherein the nucleic acid barcode includes 4-100 nucleotides in length.
11. The method of any of embodiments 1-10, wherein the nucleic acid barcode includes 10-30 nucleotides in length.
12. The method of any of embodiments 1-11, wherein the nucleic acid barcode is 18 nucleotides in length.
13. The method of any of embodiments 1-12, wherein the open reading frame encodes hemagglutinin (HA), neuraminidase (NA), M1 matrix protein (M1), M2 ion channel protein (M2), nuclear protein (NP), nonstructural protein 1 (NS1), nonstructural protein 1 (NS2), or a subunit of an RNA-dependent RNA polymerase complex selected from PB1, PB2, and PA.
14. A barcoded influenza virus genome segment including: a nucleic acid barcode and a copy of a 5′ viral RNA genome packaging signal between an end of a corresponding genome segment open reading frame and a naturally occurring non-coding portion of the 5′ viral RNA genome packaging signal wherein the copy of the 5′ viral RNA genome packaging signal has 40% to 75% sequence identity with a naturally occurring 5′ viral RNA genome packaging signal.
15. The barcoded influenza virus genome segment of embodiment 14, wherein the copy of the 5′ viral RNA genome packaging signal has about 45% to 65% sequence identity with the naturally occurring 5′ viral RNA genome packaging signal.
16. The barcoded influenza virus genome segment of embodiment 14 or 15, wherein the copy of the 5′ viral RNA genome packaging signal has about 40% to 50% sequence identity with the naturally occurring 5′ viral RNA genome packaging signal.
17. The barcoded influenza virus genome segment of any of embodiments 14-16, wherein the copy of the 5′ viral RNA genome packaging signal has about 60% to 70% sequence identity with the naturally occurring 5′ viral RNA genome packaging signal.
18. The barcoded influenza virus genome segment of any of embodiments 14-17, wherein the copy of the 5′ viral RNA genome packaging signal has 48% sequence identity with the naturally occurring 5′ viral RNA genome packaging signal.
19. The barcoded influenza virus genome segment of any of embodiments 14-18, wherein the 5′ viral RNA genome packaging signal has 62% sequence identity with the naturally occurring 5′ viral RNA genome packaging signal.
20. The barcoded influenza virus genome segment of any of embodiments 14-19, further including at least one stop codon inserted into the barcoded influenza virus genome segment.
21. The barcoded influenza virus genome segment of embodiment 20, wherein the at least one stop codon is inserted after a stop codon for the open reading frame in the copy of the 5′ viral RNA genome packaging signal.
22. The barcoded influenza virus genome segment of embodiments 20 or 21, wherein the at least one stop codon is inserted after a stop codon for the open reading frame within the 5′ viral RNA genome packaging signal that occurs after the barcode.
23. The barcoded influenza virus genome segment any of embodiments 20-22, including inserting a plurality of stop codons after a stop codon for the open reading frame within the 5′ viral RNA genome packaging signal that occurs after the barcode.
24. The barcoded influenza virus genome segment of any of embodiments 20-23, wherein the plurality of stop codons after a stop codon for the open reading frame within the 5′ viral RNA genome packaging signal that occurs after the barcode are noncontiguous.
25. The barcoded influenza virus genome segment of any of embodiments 14-24, wherein the nucleic acid barcode includes 4-100 nucleotides in length.
26. The barcoded influenza virus genome segment of any of embodiments 14-25, wherein the nucleic acid barcode includes 10-30 nucleotides in length.
27. The barcoded influenza virus genome segment of any of embodiments 14-26, wherein the nucleic acid barcode is 18 nucleotides in length.
28. The barcoded influenza virus genome segment of any of embodiments 14-27, wherein the open reading frame encodes hemagglutinin (HA), neuraminidase (NA), M1 matrix protein (M1), M2 ion channel protein (M2), nuclear protein (NP), nonstructural protein 1 (NS1), nonstructural protein 1 (NS2), or a subunit of an RNA-dependent RNA polymerase complex selected from PB1, PB2, and PA.
29. The barcoded influenza virus genome segment of any of embodiments 14-28, wherein the barcoded influenza virus genome segment is within a virion.
30. The barcoded influenza virus genome segment of the embodiment of 29, wherein the virion is an influenza virion.
31. The barcoded influenza virus genome segment of embodiments 29 or 30, wherein the influenza virion is an influenza A virion, an influenza B virion, or an influenza C virion.
32. A library of barcoded virions wherein the virions include the barcoded influenza genome segment of embodiment 14, wherein each virion's barcode is unique within the library.
33. The library of embodiment 32, wherein the library is a mutational scanning library of a viral protein.
34. The library of the embodiments of 32 or 33, wherein the library is a deep mutational scanning library of a viral protein.
35. The library of embodiment 34, wherein the viral protein is a viral entry protein.
36. The library of embodiments 33-35, wherein the viral protein is a viral fusion protein.
37. The library of embodiments 31-35, wherein the viral protein includes hemagglutinin (HA), neuraminidase (NA), M1 matrix protein (M1), M2 ion channel protein (M2), nuclear protein (NP), nonstructural protein 1 (NS1), nonstructural protein 1 (NS2), or a subunit of an RNA-dependent RNA polymerase complex selected from PB1, PB2, and PA.
38. A system including the library of barcoded virions of embodiment 32 and a control.
39. The system of embodiment 38, wherein the control is a distant antigen.
40. The system of embodiments 38 or 39, wherein the control does not react with human sera.
41. The system of any of embodiments 38-40, wherein the control includes distantly related, functional influenza hemagglutinins.
42. The system of any of embodiments 38-41, wherein the control includes a neuraminidase segment.
43. The system of any of embodiments 38-42, wherein the control does not react with human antibodies.
44. A method including: culturing virons of a library of embodiment 32; applying a selection pressure to the virions of the library; comparing growth of the virons of the library to growth of a functional standard; sequencing barcodes of variant nucleotide sequences from surviving virions of the library; and calculating a survival rate of each mutated virion of the library.
45. The method of embodiment 44, further including quantitatively measuring an impact of mutations on viral fitness in response to the selection pressure.
46. The method of embodiment 44 or 45, wherein the functional standard is a functional influenza hemagglutinin.
47. The method of any of embodiments 44-46, wherein the survival rate is used to identify a strain for vaccine development.
48. The method of any of embodiments 44-47, wherein a plurality of selection pressures are applied.
49. The method of any of embodiments 44-48, wherein the selection pressure is a putative viral neutralizing agent.
50. The method of embodiment 49, wherein the putative viral neutralizing agent includes a viral entry inhibitor and/or fusion inhibitor.
51. The method of embodiment 49 and 50, wherein the putative viral neutralizing agent includes a therapeutic compound.
52. The method of embodiment 51, wherein the therapeutic compound is undergoing pre-clinical development.
53. The method of embodiment 51, wherein the therapeutic compound is undergoing clinical development.
54. The method of any of embodiments of 51-53, wherein the therapeutic compound includes an antibody, or sera from humans or animals following infection or vaccination.
55. The method of embodiment 54, wherein the antibody is TNX-355 (ibalizumab), PGT121, or 3BNC117.
56. The method of any of embodiments 51-55, wherein the therapeutic compound includes a small molecule, a protein, a peptide, a polynucleotide, a polysaccharide, an oil, a solution, or a plant extract.
57. The method of any of embodiments 49-56, wherein dilutions of the putative neutralizing agent are applied serially.
58. The method of any of embodiments 49-57, wherein barcode counts for a given variant nucleotide sequence greater than barcode counts for the functional standard at each putative neutralizing agent concentration indicates that a virus including a viral protein encoded by the variant nucleotide sequence is resistant to the putative neutralizing agent.
59. The method of any of embodiments 49-58, wherein the selection pressure is selected from heat, cold, low pH, high pH, and a toxic agent.
60. The method of any of embodiments 49-59, wherein the selection pressure affects an ability of the virus to enter (i) a host cell of a target host species or (ii) a cell expressing a receptor protein of a species that is different from the species from which the cell was derived, wherein the ability is not dependent on presence of a functional unrelated viral entry protein.
61. The method of embodiment 60, wherein the target host species is selected from human, bat, camel, rat, and bird.
62. The method of embodiments 60 or 61, wherein the cells of the target host species are from human cell lines.
63. The method of embodiment 62, wherein the human cell lines are derived from human liver, human lung, or human lung epithelia.
64. The method of embodiments 62 or 63, wherein the human cell line derived from human liver includes HuH7, the human cell line derived from human lung includes Calu-3 or MRC-5, and/or the human cell line derived from human lung epithelia is A549 or BEAS-2B.
65. The method of embodiment 61, wherein the cells of the target host species are from bat cell lines.
66. The method of embodiment 65, wherein the bat cell lines are derived from fruit bat lung, fruit bat kidney, Egyptian fruit bat, or pipestrelle bat.
67. The method of embodiment 61, wherein the target host species is human.

(x) Experimental Examples. Example 1. Introduction. Influenza viruses evolve by rapid antigenic drift. Understanding the impact of mutations on antibody binding and escape from human neutralizing antibody-based immunity is critical to understanding fitness effects and predicting future viral evolution. Microneutralization assays are an important technique for assessing the ability of serum or antibodies to inhibit the ability of influenza viruses to infect cells. With neutralization assays on specific variants or single mutants it is possible to identify the individual mutations between two variants that have the large effects on antigenicity. However, exhaustive measurement of all combinations of mutations is highly labor intensive and often not feasible. This makes detection of mutations that have small antigenic effects or only contribute to antigenicity when observed in the background of additional mutations difficult. High-throughput methods for measuring neutralization titer against multiple strains or mutants are needed to fill this gap and thereby allow for characterization of evolutionary intermediates between antigenically distinct clusters and assessment of the impact of pairs of mutations on escape from neutralizing antibodies. A next-generation sequencing-based method which will allow for the measurement of neutralization of many virus sequences at the same time is described herein.

Discussion Using barcoded influenza, measurements of neutralization potency can be parallelized for both antibodies and serum. This method could expand the number of viruses that can be tested in serology studies with small volumes of serum. With a high-throughput method for measuring neutralization titer against multiple strains at once, one could characterize evolutionary intermediates between antigenically distinct variants or assess how combinations of mutations impact escape from human serum neutralizing antibodies.

Example 2. Influenza A viruses evolve by rapid antigenic drift. Understanding the impact of mutations on antibody binding and escape from human neutralizing immunity is critical to understanding fitness effects and predicting future viral evolution.

With neutralization assays on specific variants or single mutants it is possible to identify the individual mutations between two variants that have the large effects on antigenicity. However, exhaustive measurement of all combinations of mutations is highly labor intensive and often not feasible. This limits detection of mutations that have small antigenic effects or only contribute to antigenicity when observed in the background of additional mutations. High-throughput methods for measuring neutralization titer against multiple strains or mutants are needed to fill this gap and thereby allow for characterization of evolutionary intermediates between antigenically distinct clusters and assessment of the impact of pairs of mutations on escape from putative neutralizing agents such as neutralizing antibodies or other therapeutic compounds.

Here, a next-generation sequencing (NGS)-based method is described which will allow for the measurement of neutralization of many virus sequences at the same time. This method relies on the incorporation of barcode sequences into the hemagglutinin (HA) segment of the influenza genome.

The technology includes a novel design for incorporating a nucleotide barcode into influenza gene segments such that libraries of influenza virions can be generated which each carry a barcode that is linked to a different viral protein sequence. By sequencing the barcode, it is possible to identify the full sequence of the viral gene. These libraries can then be used with large-scale sequencing technologies (NGS) to make massively parallel measurements of how mutations to the viral proteins affect viral growth and immune recognition.

Methods to create barcoded influenza viruses without disrupting the function of the viral proteins and the proper packaging of the viral genome segments are described. The barcoded influenza viruses can be used within deep mutational scanning libraries to map influenza resistance mutations to therapeutic treatments and can be used to make parallel measurements against a defined set of recently circulating or historically relevant influenza strains. The libraries can also be used to predict influenza strains that may become resistant to therapeutic treatments and/or more easily evolve to infect new species. The libraries include features that allow efficient collection and assessment of informative data.

This design for incorporating barcodes into the influenza gene segments is significantly including improved barcode retention and stability. Specifically, the sequence identity of the internal duplicated region of the packaging signal is low compared the terminal packaging region to limit homologous recombination and consequent loss of barcodes. For example, the sequence identity may be between 40% to 75%, 45% to 70%, 50% to 60%, 40%, 42%, 45%, 48% 60%, 62% 68% or any integers in between.

In particular embodiments, the sequence similarity between the internal duplicate region of the packaging signal is 40%-75% sequence similarity for H3 constructs, for example 40% to 75%, 45% to 70%, 50% to 60%, 40%, 42%, 45%, 48% 60%, 62% 68% or any integers in between. In particular embodiments, the sequence similarity between the internal duplicate region of the packaging signal is 48% sequence similarity for H3 constructs. In particular embodiments, the sequence similarity between the internal duplicate region of the packaging signal is 40%-100% sequence similarity for H1 construct, for example 40% to 75%, 45% to 70%, 50% to 60%, 40%, 42%, 45%, 48% 60%, 62% 68% or any integers in between. In particular embodiments, the sequence similarity between the internal duplicate region of the packaging signal is 62% sequence similarity for H1 constructs.

Additionally, one (or multiple) stop codons are incorporated in the coding region of the terminal packaging signal such that if barcode region is deleted, the non-barcoded construct is less likely to produce functional virions. In particular embodiments, the stop codons are incorporated after a stop codon for the open reading frame in the copy of a 5′ viral RNA genome packaging signal. This method has been tested with both H1 and H3 influenza strains.

In addition, barcoded genomic segments were designed for distantly related, TC-adapted, functional influenza hemagglutinins (HAs) that can be used as internal standards for experiments with barcoded influenza. Most humans have limited neutralization activity against these distant HAs. When performing selection experiments, the relative growth of library variants were analyzed in the presence and absence of a selective pressure. Including a distant HA as an internal standard for non-neutralized virus growth allows for quantitative measurement of the impact of mutations on neutralization. For example, cultured virons may be exposed to a neutralizing agent and the growth of the viron compared to the distant HA allowing for the calculation of the survival rate of each mutated viron. The barcodes of the variant nucleotide sequences of the surviving virons may be sequenced, allowing for the calculation of a survival rate of each mutated virion of the library.

In addition, the relative frequencies of mutants or variants can be used with respect to this control at various concentrations to calculate a measurement akin to an IC50 (half maximal inhibitory concentration) from a neutralization assay (which is currently the standard approach for assessing inhibition of infection by serum or antibodies). This system was developed so that large-scale sequencing technologies (NGS) can execute massively parallel neutralization assays with the barcoded influenza variant libraries. With this method, IC50-like measurements can be generated for hundreds of viruses at once, using the same volume of sample that is currently used to generate an IC50 against a single virus, or tens of thousands of variants when larger volumes of serum are available. This advancement will allow for the generation of significantly more measurements and gain more detailed information about immune specificity of a given sample against many viruses, even for samples which have limited volume.

(xi) Closing Paragraphs. The nucleic acid and amino acid sequences provided herein are shown using letter abbreviations for nucleotide bases and amino acid residues, as defined in 37 C.F.R. § 1.831-1.835 and set forth in WIPO Standard ST.26 (implemented on Jul. 1, 2022). Only one strand of each nucleic acid sequence is shown, but the complementary strand is understood as included in embodiments where it would be appropriate.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (nonpolar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157 (1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y., Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of a binding domain (of, for example, a CAR binding domain or a nanoparticle selected cell targeting ligand) to its cognate binding molecule with an affinity or Ka (i.e., an equilibrium association constant of a particular binding interaction with units of 1/M) equal to or greater than 10⁵ M⁻¹, while not significantly associating with any other molecules or components in a relevant environment sample. “Specifically binds” is also referred to as “binds” herein. Binding domains may be classified as “high affinity” or “low affinity”. In particular embodiments, “high affinity” binding domains refer to those binding domains with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding domains refer to those binding domains with a Ka of up to 10⁷ M⁻¹, up to 10⁶ M⁻¹, up to 10⁵ M⁻¹. Alternatively, affinity may be defined as an equilibrium dissociation constant (Kd) of a particular binding interaction with units of M (e.g., 10⁻⁵ M to 10⁻¹³ M). In certain embodiments, a binding domain may have “enhanced affinity,” which refers to a selected or engineered binding domains with stronger binding to a cognate binding molecule than a wild type (or parent) binding domain. For example, enhanced affinity may be due to a Ka (equilibrium association constant) for the cognate binding molecule that is higher than the reference binding domain or due to a Kd (dissociation constant) for the cognate binding molecule that is less than that of the reference binding domain, or due to an off-rate (Koff) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would result in an increase in loss of barcodes with uninhibited survival of virions without barcodes.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, other written text, and web site content throughout this specification (referenced materials herein). Each of the referenced materials are individually incorporated herein by reference in their entirety for their referenced teaching(s), as of the filing date of the first application in the priority chain in which the specific reference was included. For instance, with regard to chemical compounds, nucleic acid, and amino acids sequences referenced herein that are available in a public database, the information in the database entry is incorporated herein by reference as of the date of an application in the priority chain in which the database identifier for that compound or sequence was first included in the text.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A method for barcoding an influenza virus genome segment comprising:
inserting a nucleic acid barcode and a copy of a coding region of a 5′ viral RNA genome packaging signal between a terminus of a corresponding genome segment open reading frame and a naturally occurring non-coding portion of the 5′ viral RNA genome packaging signal; and
inserting at least one stop codon in the influenza virus genome segment;
wherein the copy of the coding region of the 5′ viral RNA genome packaging signal has 40% to 75% sequence identity with a naturally occurring 5′ viral RNA genome packaging signal.

2. The method of claim 1, wherein the copy of the coding region of the 5′ viral RNA genome packaging signal has about 45% to 65% sequence identity with the naturally occurring 5′ viral RNA genome packaging signal.

3. The method of claim 1, wherein the copy of the coding region of the 5′ viral RNA genome packaging signal has about 40% to 50% sequence identity with the naturally occurring 5′ viral RNA genome packaging signal.

4. The method of claim 1, wherein the copy of the coding region of the 5′ viral RNA genome packaging signal has about 60% to 70% sequence identity with the naturally occurring 5′ viral RNA genome packaging signal.

5. The method of claim 1, wherein the copy of the coding region of the 5′ viral RNA genome packaging signal has 48% sequence identity with the naturally occurring 5′ viral RNA genome packaging signal.

6. The method of claim 1, wherein the coding region of the 5′ viral RNA genome packaging signal has 62% sequence identity with the naturally occurring 5′ viral RNA genome packaging signal.

7. The method of claim 1, wherein the at least one stop codon is inserted after a stop codon for the open reading frame within the 5′ viral RNA genome packaging signal that occurs after the barcode.

8. The method of claim 1, comprising inserting a plurality of stop codons after a stop codon for the open reading frame within the 5′ viral RNA genome packaging signal that occurs after the barcode.

9. The method of claim 8, wherein the plurality of stop codons after a stop codon for the open reading frame within the 5′ viral RNA genome packaging signal that occurs after the barcode are noncontiguous.

10. The method of claim 1, wherein the nucleic acid barcode comprises 4-100 nucleotides in length.

11. The method of claim 1, wherein the nucleic acid barcode comprises 10-30 nucleotides in length.

12. The method of claim 1, wherein the nucleic acid barcode is 18 nucleotides in length.

13. The method of claim 1, wherein the open reading frame encodes hemagglutinin (HA), neuraminidase (NA), M1 matrix protein (M1), M2 ion channel protein (M2), nuclear protein (NP), nonstructural protein 1 (NS1), nonstructural protein 1 (NS2), or a subunit of an RNA-dependent RNA polymerase complex selected from PB1, PB2, and PA.

14. A barcoded influenza virus genome segment comprising:
a nucleic acid barcode and a copy of a 5′ viral RNA genome packaging signal between an end of a corresponding genome segment open reading frame and a naturally occurring non-coding portion of the 5′ viral RNA genome packaging signal wherein the copy of the 5′ viral RNA genome packaging signal has 40% to 75% sequence identity with a naturally occurring 5′ viral RNA genome packaging signal.

15. The barcoded influenza virus genome segment of claim 14, wherein the copy of the 5′ viral RNA genome packaging signal has about 45% to 65% sequence identity with the naturally occurring 5′ viral RNA genome packaging signal.

16. The barcoded influenza virus genome segment of claim 14, wherein the copy of the 5′ viral RNA genome packaging signal has about 40% to 50% sequence identity with the naturally occurring 5′ viral RNA genome packaging signal.

17. The barcoded influenza virus genome segment of claim 14, wherein the copy of the 5′ viral RNA genome packaging signal has about 60% to 70% sequence identity with the naturally occurring 5′ viral RNA genome packaging signal.

18. The barcoded influenza virus genome segment of claim 14, wherein the copy of the 5′ viral RNA genome packaging signal has 48% sequence identity with the naturally occurring 5′ viral RNA genome packaging signal.

19. The barcoded influenza virus genome segment of claim 14, wherein the 5′ viral RNA genome packaging signal has 62% sequence identity with the naturally occurring 5′ viral RNA genome packaging signal.

20. The barcoded influenza virus genome segment of claim 14, further comprising at least one stop codon inserted into the barcoded influenza virus genome segment.

21. The barcoded influenza virus genome segment of claim 20, wherein the at least one stop codon is inserted after a stop codon for the open reading frame in the copy of the 5′ viral RNA genome packaging signal.

22. The barcoded influenza virus genome segment of claim 20, wherein the at least one stop codon is inserted after a stop codon for the open reading frame within the 5′ viral RNA genome packaging signal that occurs after the barcode.

23. The barcoded influenza virus genome segment of claim 20, comprising inserting a plurality of stop codons after a stop codon for the open reading frame within the 5′ viral RNA genome packaging signal that occurs after the barcode.

24. The barcoded influenza virus genome segment of claim 23, wherein the plurality of stop codons after a stop codon for the open reading frame within the 5′ viral RNA genome packaging signal that occurs after the barcode are noncontiguous.

25. The barcoded influenza virus genome segment of claim 21, wherein the nucleic acid barcode comprises 4-100 nucleotides in length.

26. The barcoded influenza virus genome segment of claim 21, wherein the nucleic acid barcode comprises 10-30 nucleotides in length.

27. The barcoded influenza virus genome segment of claim 21, wherein the nucleic acid barcode is 18 nucleotides in length.

28. The barcoded influenza virus genome segment of claim 21, wherein the open reading frame encodes hemagglutinin (HA), neuraminidase (NA), M1 matrix protein (M1), M2 ion channel protein (M2), nuclear protein (NP), nonstructural protein 1 (NS1), nonstructural protein 1 (NS2), or a subunit of an RNA-dependent RNA polymerase complex selected from PB1, PB2, and PA.

29. The barcoded influenza virus genome segment of claim 21, wherein the barcoded influenza virus genome segment is within a virion.

30. The barcoded influenza virus genome segment of claim 29, wherein the virion is an influenza virion.

31. The barcoded influenza virus genome segment of claim 29, wherein the influenza virion is an influenza A virion, an influenza B virion, or an influenza C virion.

32. A library of barcoded virions wherein the virions comprise the barcoded influenza genome segment of claim 14, wherein each virion's barcode is unique within the library.

33. The library of claim 32, wherein the library is a mutational scanning library of a viral protein.

34. The library of claim 33, wherein the library is a deep mutational scanning library of a viral protein.

35. The library of claim 34, wherein the viral protein is a viral entry protein.

36. The library of claim 34, wherein the viral protein is a viral fusion protein.

37. The library of claim 34, wherein the viral protein comprises hemagglutinin (HA), neuraminidase (NA), M1 matrix protein (M1), M2 ion channel protein (M2), nuclear protein (NP), nonstructural protein 1 (NS1), nonstructural protein 1 (NS2), or a subunit of an RNA-dependent RNA polymerase complex selected from PB1, PB2, and PA.

38. A system comprising the library of barcoded virions of claim 32 and a control.

39. The system of claim 38, wherein the control is a distant antigen.

40. The system of claim 38, wherein the control does not react with human sera.

41. The system of claim 40, wherein the control comprises distantly related, functional influenza hemagglutinins.

42. The system of claim 40, wherein the control comprises a neuraminidase segment.

43. The system of claim 38, wherein the control does not react with human antibodies.

44. A method comprising:
culturing virons of the library of claim 32;
applying a selection pressure to the virions of the library;
comparing growth of the virons of the library to growth of a functional standard;
sequencing barcodes of variant nucleotide sequences from surviving virions of the library; and
calculating a survival rate of each mutated virion of the library.

45. The method of claim 44, further comprising quantitatively measuring an impact of mutations on viral fitness in response to the selection pressure.

46. The method of claim 44, wherein the functional standard is a functional influenza hemagglutinin.

47. The method of claim 44, wherein the survival rate is used to identify a strain for vaccine development.

48. The method of claim 44, wherein a plurality of selection pressures are applied.

49. The method of claim 44, wherein the selection pressure is a putative viral neutralizing agent.

50. The method of claim 49, wherein the putative viral neutralizing agent comprises a viral entry inhibitor and/or fusion inhibitor.

51. The method of claim 49, wherein the putative viral neutralizing agent comprises a therapeutic compound.

52. The method of claim 51, wherein the therapeutic compound is undergoing pre-clinical development.

53. The method of claim 51, wherein the therapeutic compound is undergoing clinical development.

54. The method of claim 51, wherein the therapeutic compound comprises an antibody, or sera from humans or animals following infection or vaccination.

55. The method of claim 54, wherein the antibody is TNX-355 (ibalizumab), PGT121, or 3BNC117.

56. The method of claim 51, wherein the therapeutic compound comprises a small molecule, a protein, a peptide, a polynucleotide, a polysaccharide, an oil, a solution, or a plant extract.

57. The method of claim 49, wherein dilutions of the putative neutralizing agent are applied serially.

58. The method of claim 49, wherein barcode counts for a given variant nucleotide sequence greater than barcode counts for the functional standard at each putative neutralizing agent concentration indicates that a virus comprising a viral protein encoded by the variant nucleotide sequence is resistant to the putative neutralizing agent.

59. The method of claim 48, wherein the selection pressure is selected from heat, cold, low pH, high pH, and a toxic agent.

60. The method of claim 48, wherein the selection pressure affects an ability of the virus to enter (i) a host cell of a target host species or (ii) a cell expressing a receptor protein of a species that is different from the species from which the cell was derived, wherein the ability is not dependent on presence of a functional unrelated viral entry protein.

61. The method of claim 60, wherein the target host species is selected from human, bat, camel, rat, and bird.

62. The method of claim 60, wherein the cells of the target host species are from human cell lines.

63. The method of claim 62, wherein the human cell lines are derived from human liver, human lung, or human lung epithelia.

64. The method of claim 63, wherein the human cell line derived from human liver comprises HuH7, the human cell line derived from human lung comprises Calu-3 or MRC-5, and/or the human cell line derived from human lung epithelia is A549 or BEAS-2B.

65. The method of claim 62, wherein the cells of the target host species are from bat cell lines.

66. The method of claim 65, wherein the bat cell lines are derived from fruit bat lung, fruit bat kidney, Egyptian fruit bat, or pipestrelle bat.

67. The method of claim 60, wherein the target host species is human.


SEQUENCE LISTING (45 sequences)

SEQ ID NO: 1 - RNA, 99 nt, Influenza A virus
  source 1..99: mol_type = other RNA
agcaaaagcaggggaaaataaaaacaaccaaattgaaggcaaacctactggtcctgttaagtgcacttgcagctgcagttgcagacacaatttgtatag

SEQ ID NO: 2 - skipped

SEQ ID NO: 3 - RNA, 173 nt, Influenza A virus
  source 1..173: mol_type = other RNA
agcgaaagcaggggtttaaattgaatccaaatcagaaaataacaaccattggatcaatctgtctggtagtcggactaattagcctaatattgcaaatagggaatataatctcaatttggattagccattcaattcaaactggaagtcaaaaccatactggaatttgcaaccaa

SEQ ID NO: 4 - skipped

SEQ ID NO: 5 - RNA, 1778 nt, Influenza A virus
  source 1..1778: mol_type = viral cRNA; strain = A/Puerto Rico/8/1934(H1N1)
agcaaaagcaggggaaaataaaaacaaccaaaatgaaggcaaacctactggtcctgttatgtgcacttgcagctgcagatgcagacacaatatgtataggctaccatgcgaacaattcaaccgacactgttgacacagtgctcgagaagaatgtgacagtgacacactctgttaacctgctcgaagacagccacaacggaaaactatgtagattaaaaggaatagccccactacaattggggaaatgtaacatcgccggatggctcttgggaaacccagaatgcgacccactgcttccagtgagatcatggtcctacattgtagaaacaccaaactctgagaatggaatatgttatccaggagatttcatcgactatgaggagctgagggagcaattgagctcagtgtcatcattcgaaagattcgaaatatttcccaaagaaagctcatggcccaaccacaacacaaccaaaggagtaacggcagcatgctcccatgcggggaaaagcagtttttacagaaatttgctatggctgacggagaaggagggctcatacccaaagctgaaaaattcttatgtgaacaagaaagggaaagaagtccttgtactgtggggtattcatcacccgtctaacagtaaggatcaacagaatatctatcagaatgaaaatgcttatgtctctgtagtgacttcaaattataacaggagatttaccccggaaatagcagaaagacccaaagtaagagatcaagctgggaggatgaactattactggaccttgctaaaacccggagacacaataatatttgaggcaaatggaaatctaatagcaccaaggtatgctttcgcactgagtagaggctttgggtccggcatcatcacctcaaacgcatcaatgcatgagtgtaacacgaagtgtcaaacacccctgggagctataaacagcagtctccctttccagaatatacacccagtcacaataggagagtgcccaaaatacgtcaggagtgccaaattgaggatggttacaggactaaggaacattccgtccattcaatccagaggtctatttggagccattgccggttttattgaagggggatggactggaatgatagatggatggtacggttatcatcatcagaatgaacagggatcaggctatgcagcggatcaaaaaagcacacaaaatgccattaacgggattacaaacaaggtgaactctgttatcgagaaaatgaacattcaattcacagctgtgggtaaagaattcaacaaattagaaaaaaggatggaaaatttaaataaaaaagttgatgatggatttctggacatttggacatataatgcagaattgttagttctactggaaaatgaaaggactctggatttccatgactcaaatgtgaagaatctgtatgagaaagtaaaaagccaattaaagaataatgccaaagaaatcggaaatggatgttttgagttctaccacaagtgtgacaatgaatgcatggaaagtgtaagaaatgggacttatgattatcccaaatattcagaagagtcaaagttgaacagggaaaaggtagatggagtgaaattggaatcaatggggatctatcagattctggcgatctactcaactgtcgccagttcactggtgcttttggtctccctgggggcaatcagtttctggatgtgttctaatggatctttgcagtgcagaatatgcatctgagattagaatttcagaaatatgaggaaaaacacccttgtttctact

SEQ ID NO: 6 - RNA, 1413 nt, Influenza A virus
  source 1..1413: mol_type = viral cRNA; strain = A/Puerto Rico/8/1934(H1N1)
agcgaaagcaggggtttaaaatgaatccaaatcagaaaataataaccattggatcaatctgtctggtagtcggactaattagcctaatattgcaaatagggaatataatctcaatatggattagccattcaattcaaactggaagtcaaaaccatactggaatatgcaaccaaaacatcattacctataaaaatagcacctgggtaaaggacacaacttcagtgatattaaccggcaattcatctctttgtcccatccgtgggtgggctatatacagcaaagacaatagcataagaattggttccaaaggagacgtttttgtcataagagagccctttatttcatgttctcacttggaatgcaggaccttttttctgacccaaggtgccttactgaatgacaggcattcaaatgggactgttaaggacagaagcccttatagggccttaatgagctgccctgtcggtgaagctccgtccccgtacaattcaagatttgaatcggttgcttggtcagcaagtgcatgtcatgatggcatgggctggctaacaatcggaatttcaggtccagataatggagcagtggctgtattaaaatacaacggcataataactgaaaccataaaaagttggaggaagaaaatattgaggacacaagagtctgaatgtgcctgtgtaaatggttcatgttttactataatgactgatggcccgagtgatgggctggcctcgtacaaaattttcaagatcgaaaaggggaaggttactaaatcaatagagttgaatgcacctaattctcactatgaggaatgttcctgttaccctgataccggcaaagtgatgtgtgtgtgcagagacaattggcatggttcgaaccggccatgggtgtctttcgatcaaaacctggattatcaaataggatacatctgcagtggggttttcggtgacaacccgcgtcccaaagatggaacaggcagctgtggtccagtgtatgttgatggagcaaacggagtaaagggattttcatataggtatggtaatggtgtttggataggaaggaccaaaagtcacagttccagacatgggtttgagatgatttgggatcctaatggatggacagagactgatagtaagttctctgtgaggcaagatgttgtggcaatgactgattggtcagggtatagcgggagtttcgttcaacatcctgagctaacagggctagactgtataaggccgtgcttctgggttgaattaatcaggggacgacctaaagaaaaaacaatctggactagtgcgagcagcatttctttttgtggcgtgaatagtgatactgtagattggtcttggccagacggtgctgagttgccattcaccattgacaagtagtctgttcaaaaaactccttgtttctact

SEQ ID NO: 7 - RNA, 1762 nt, Influenza A virus
  source 1..1762: mol_type = viral cRNA; strain = A/New York/392/2004(H3N2)
agcaaaagcaggggataattctattaaccatgaagactatcattgctttgagctacattctatgtctggttttcgctcaaaaacttcccggaaatgacaacagcacggcaacgctgtgccttgggcaccatgcagtaccaaacggaacgatagtgaaaacaatcacgaatgaccaaattgaagtcactaatgctactgaactggttcagagttcctcaacaggtggaatatgcgacagtcctcatcagatccttgatggagaaaactgcacactaatagatgctctattgggagaccctcagtgtgatggcttccaaaataagaaatgggacctttttgttgaacgcagcaaagcctacagcaactgttacccttatgatgtgccggattatgcctcccttaggtcactagttgcctcatccggcacactggagtttaacaatgaaagcttcaattggactggagtcactcaaaatggaacaagctctgcttgcaaaaggagatctaataacagtttctttagtagattgaattggttgacccacttaaaattcaaatacccagcattgaacgtgactatgccaaacaatgaaaaatttgacaaactgtacatttggggggttcaccacccgggtacggacaatgaccaaatcagcctatatgctcaagcatcaggaagaatcacagtctctaccaaaagaagccaacaaaccgtaatcccgagtatcggatctagacccaggataagggatgtccccagcagaataagcatctattggacaatagtaaaaccgggagacatacttttgattaacagcacagggaatctaattgctcctcggggttacttcaaaatacgaagtgggaaaagctcaataatgagatcagatgcacccattggcaaatgcaattctgaatgcatcactccaaatggaagcattcccaatgacaaaccatttcaaaatgtaaacaggatcacatatggggcctgtcccagatatgttaagcaaaacactctgaaattggcaacagggatgcgaaatgtaccagagaaacaaactagaggcatatttggcgcaatcgcgggtttcatagaaaatggttgggagggaatggtagacggttggtacggtttcaggcatcaaaattctgagggaacaggacaagcagcagatctcaaaagcactcaagcagcaatcaaccaaatcaatgggaagctgaataggttgatcgggaaaacaaacgagaaattccatcagattgaaaaagaattctcagaagtagaagggagaattcaggacctcgagaaatatgttgaggacactaaaatagatctctggtcatacaacgcggagcttcttgtggccctggagaaccaacatacaattgatctaactgactcagaaatgaacaaactgtttgaaagaacaaagaagcaactgagggaaaatgctgaggatatgggcaatggttgtttcaaaatataccacaaatgtgacaatgcctgcatagggtcaatcagaaatggaacttatgaccatgatgtatacagagatgaagcattaaacaaccggttccagatcaaaggtgttgagttgaagtcaggatacaaagattggatcctatggatttcctttgccatatcatgttttttgctttgtgttgctttgttggggttcatcatgtgggcctgccaaaaaggcaacattaggtgcaacatttgcatttgagtgcattaattaaaaacacccttgtttctact

SEQ ID NO: 8 - RNA, 1467 nt, Influenza A virus
  source 1..1467: mol_type = viral cRNA; strain = A/New York/392/2004(H3N2)
agcaaaagcaggagtaaagatgaatccaaatcaaaagataataacgattggctctgtttctctcaccatttccacaatatgcttcttcatgcaaattgccatcctgataaccactgtaacattgcatttcaagcaatatgaattcaactcccccccaaacaaccaagtgatgctgtgtgaaccaacaataatagaaagaaacataacagagatagtgtatctgaccaacaccaccatagagaaggaaatgtgccccaaactagcagaatacagaaattggtcaaagccgcaatgtgacattacaggatttgcacctttttctaaggacaattcgattaggctttccgctggtggggacatctgggtgacaagagaaccttatgtgtcatgcgaccctgacaagtgttaccaatttgcccttggacagggaacaacactaaacaacgtgcattcaaatgacacagtacatgataggaccccttatcggaccctattgatgaatgaattaggtgttccatttcatctggggaccaagcaagtgtgcatagcatggtccagctcaagttgtcacgatggaaaagcatggctgcatgtttgtgtaacgggggatgataaaaatgcaactgctagcttcatttacaatgggaggcttgtagatagtattgtttcatggtccaaaaaaatcctcaggacccaggagtcagaatgcgtttgtatcaatggaacttgtacagtagtaatgactgatgggagtgcttcaggaaaagctgatactaaaatactattcattgaggaggggaaaatcattcatactagcacattgtcaggaagtgctcagcatgtcgaggagtgctcctgctatcctcgatatcctggtgtcagatgtgtctgcagagacaactggaaaggctccaataggcccatcgtagatataaacataaaggattatagcattgtttccagttatgtgtgctcagggcttgttggagacacacccagaaaaaacgacagctccagcagtagccattgcttggatcctaacaatgaagaaggtggtcatggagtgaaaggctgggcctttgatgatggaaatgacgtgtggatgggaagaacgatcagcgagaagttacgctcaggatatgaaaccttcaaagtcattgaaggctggtccaaacctaattccaaattgcagataaataggcaagtcatagttgacagaggtaataggtccggttattctggtattttctctgttgaaggcaaaagctgcatcaatcggtgcttttatgtggagttgataaggggaagaaaagaggaaactgaagtcttgtggacctcaaacagtattgttgtgttttgtggcacctcaggtacatatggaacaggctcatggcctgatggggcggacatcaatctcatgcctatataagctttcgcaattttagaaaaaaactccttgtttctact

SEQ ID NO: 9 - RNA, 1760 nt, Influenza A virus
  source 1..1760: mol_type = viral cRNA; strain = A/goose/Guangdong/1/1996(H5N1)
gcaggggtataatctgtcaaaatggagaaaatagtgcttcttcttgcaatagtcagtcttgtcaaaagtgatcagatttgcattggttaccatgcaaacaactcgacagagcaggttgacacaataatggaaaagaacgttactgttacacatgcccaagacatactggaaaagacacacaatgggaagctctgcgatctaaatggagtgaagcctctcattttgagagattgtagtgtagctggatggctcctcggaaaccctatgtgtgacgaattcatcaatgtgccggaatggtcttacatagtggagaaggccagtccagccaatgacctctgttacccaggggatttcaacgactatgaagaactgaaacacctattgagcagaacaaaccattttgagaaaattcagatcatccccaaaagttcttggtccaatcatgatgcctcatcaggggtgagctcagcatgtccataccatgggaggtcctcctttttcagaaatgtggtatggcttatcaaaaagaacagtgcatacccaacaataaagaggagctacaataataccaaccaagaagatcttttagtactgtgggggattcaccatcctaatgatgcggcagagcagacaaagctctatcaaaacccaaccacttacatttccgttggaacatcaacactgaaccagagattggttccagaaatagctactagacccaaagtaaacgggcaaagtggaagaatggagttcttctggacaattttaaagccgaatgatgccatcaatttcgagagtaatggaaatttcattgctccagaatatgcatacaaaattgtcaagaaaggggactcagcaattatgaaaagtgaattggaatatggtaactgcaacaccaagtgtcaaactccaatgggggcgataaactctagtatgccattccacaacatacaccccctcaccatcggggaatgccccaaatatgtgaaatcaaacagattagtccttgcgactggactcagaaatacccctcagagagagagaagaagaaaaaagagaggactatttggagctatagcaggttttatagagggaggatggcagggaatggtagatggttggtatgggtaccaccatagcaatgagcaggggagtggatacgctgcagacaaagaatccactcaaaaggcaatagatggagtcaccaataaggtcaactcgatcattgacaaaatgaacactcagtttgaggccgttggaagggaatttaataacttggaaaggaggatagagaatttaaacaagcagatggaagacggattcctagatgtctggacttataatgctgaacttctggttctcatggaaaatgagagaactctagactttcatgactcaaatgtcaagaacctttatgacaaggtccgactacagcttagggataatgcaaaggagctgggtaatggttgtttcgagttctatcacaaatgtgataatgaatgtatggaaagtgtaaaaaacggaacgtatgactacccgcagtattcagaagaagcaagactaaacagagaggaaataagtggagtaaaattggaatcaatgggaacttaccaaatactgtcaatttattcaacagtggcgagttccctagcactggcaatcatggtagctggtctatctttatggatgtgctccaatggatcgttacaatgcagaatttgcatttaaatttgtgagttcagattgtagttaaaaacacc

SEQ ID NO: 10 - RNA, 1458 nt, Influenza A virus
  source 1..1458: mol_type = viral cRNA; strain = A/Goose/Guangdong/1/96(H5N1)
agcaaaagcaggagattaaaatgaatccaaatcagaagataataaccattggatcaatctgtatggtagttgggataattagcttgatgttacaaattgggaacataatctcaatatgggtcagtcattcaattcagacagggaatcaacaccaagctgaaccatgcaatcaaagcattattacttatgaaaacaacacctgggtaaatcaaacatatgtcaacatcagcaataccaattttcttactgaaaaagctgtggcttcagtaacattagcgggcaattcatctctttgccccattagcggatgggctgtacacagtaaggacaacggtataagaatcggttccaagggggatgtgtttgttataagagagccgttcatctcatgctcccacttggaatgcagaactttctttttgactcagggagccttgctgaatgacaagcactccaatgggaccgtcaaagacagaagccctcacagaacattgatgagttgtcctgtgggtgaggctccctccccatataactcaaggtttgagtctgttgcttggtcggcaagtgcttgccatgatggcaccagttggttgacaattggaatttctggcccagacaatggggctgtggctgtattgaaatacaacggcataataacagacactatcaagagttggaggaacaacatactgagaactcaagagtctgaatgtgcatgtgtaaatggctcttgctttactgtaatgactgacggaccaagtaatgggcaggcctcatataagatcttcaaaatggaaaaagggaaagtagttaaatcagtcgaattgaatgcccctaattatcactatgaggagtgctcctgttatcctgatgctggcgaaatcacatgtgtgtgcagggataattggcatggctcaaatcggccatgggtatctttcaatcaaaatttggagtatcaaataggatatatatgcagtggagttttcggagacaatccacgccccaatgatggaacaggcagttgtggtccggtgtcccctaacggggcatatggagtaaaagggttttcatttaaatacggcaatggtgtttggatcgggagaaccaaaagcactaattccaggagcggctttgaaatgatttgggatccaaatgggtggactggaacggacagtagcttctcggtgaaacaagatatcgtagcaataactgattggtcaggatatagcgggagttttgtccagcatccagaactgacaggattagattgcataagaccttgtttctgggttgagctaatcagagggcggcccaaagagagcacaatttggactagtgggagcagcatatctttttgtggtgtaaatagtgacactgtgggttggtcttggccagacgatgccgagttgccattcaccattgacaagtagtttgttcaaaaaactccttgtttctact

SEQ ID NO: 11 - RNA, 1882 nt, Influenza B virus
  source 1..1882: mol_type = viral cRNA; strain = B/Lee/1940
agcagaagcgttgcattttctaatatccacaaaatgaaggcaataattgtactactcatggtagtaacatccaatgcagatcgaatctgcactgggataacatcgtcaaactcacctcatgtggttaaaactgccactcaaggggaagtcaatgtgactggtgtgataccactaacaacaacacctaccaaatctcattttgcaaatctcaaaggaacacagaccagaggaaaactatgcccaaactgttttaactgcacagatctggacgtggccctaggcagaccaaaatgcatggggaacacaccctccgcaaaagtctcaatactccatgaagtcaaacctgctacatctggatgctttcctataatgcacgacagaacaaaaatcagacaactacctaatcttctcagaggatatgaaaacatcaggttatcaaccagtaatgttatcaatacagagacggcaccaggaggaccctacaaggtggggacctcaggatcttgccctaacgttgctaatgggaacggcttcttcaacacaatggcttgggttatcccaaaagacaacaacaagacagcaataaatccagtaacagtagaagtaccatacatttgttcagaaggggaagaccaaattactgtttgggggttccactctgatgacaaaacccaaatggaaagactctatggagactcaaatcctcaaaagttcacctcatctgccaatggagtaaccacacattatgtttctcagattggtggcttcccaaatcaaacagaagacgaagggctaaaacaaagcggcagaattgttgttgattacatggtacaaaaacctggaaaaacaggaacaattgtttatcaaagaggcattttattgcctcaaaaagtgtggtgcgcaagtggcaggagcaaggtaataaaagggtccttgcctttaattggtgaagcagattgcctccacgaaaagtacggtggattaaataaaagcaagccttactacacaggagagcatgcaaaggccataggaaattgcccaatatgggtgaaaacacccttgaagctggccaatggaaccaaatatagaccgcctgcaaaactattaaaggaaagaggtttcttcggagctattgctggtttcttggaaggaggatgggaaggaatgattgcaggttggcacggatacacatctcatggagcacatggagtggcagtggcagcagaccttaagagtacacaagaagctataaacaagataacaaaaaatctcaactatttaagtgagctagaagtaaaaaaccttcaaagactaagcggagcaatgaatgagcttcacgacgaaatactcgagctagacgaaaaagtggatgatctaagagctgatacaataagctcacaaatagagcttgcagtcttgctttccaacgaagggataataaacagtgaagatgagcatctcttggcacttgaaagaaaactgaagaaaatgcttggcccctctgctgtagaaatagggaatgggtgctttgaaaccaaacacaaatgcaaccagacttgcctagacaggatagctgctggcacctttaatgcaggagatttttctcttcccacttttgattcattaaacattactgctgcatctttaaatgatgatggcttggataatcatactatactgctctactactcaactgctgcttctagcttggctgtaacattaatgatagctatcttcattgtctacatggtctccagagacaatgtttcttgttccatctgtctgtgagggagattaagccctgtgttttcctttactgtagtgctcatttgcttgtcaccattacaaagaaacgttattgaaaaatgctcttgttactact

SEQ ID NO: 12 - RNA, 1557 nt, Influenza B virus
  source 1..1557: mol_type = viral cRNA; strain = B/Lee/1940
agcagaagcagagcatattcttagaactgaagtgaacaggccaaaaatgaacaatgctaccttcaactgtacaaacattaaccctattactcacatcagggggagtattattatcactatatgtgtcagcctcattgtcatacttattgtattcggatgtattgctaaaattttcatcaacaaaaacaactgcaccaacaatgtcattagagtgcacaaacgcatcaaatgcccagactgtgaaccattctgcaacaaaagagatgacatttccacccccagagccggagtggacataccctcgtttatcttgccagggctcaacctttcagaaggcactcctaattagccctcataggttcggagagatcaaaggaaactcagctcccttgataataagagaaccttttgttgcttgtggaccaaaagaatgcagacactttgctctgacccattatgcagctcagccggggggatactacaatggaacaagaaaggacagaaacaagctgaggcatctagtatcagtcaaattgggaaaaatcccaactgtggaaaactccattttccacatggcagcttggagcggatccgcatgccatgatggtagagaatggacatatatcggagttgatggtcctgacaatgatgcattggtcaaaataaaatatggagaagcatatactgacacatatcattcctatgcacacaacatcctaagaacacaagaaagtgcctgcaattgcatcgggggagattgttatcttatgataacagacggctcagcttcaggaattagtaaatgcagatttcttaaaattagagagggtcgaataataaaagaaatacttccaacaggaagagtggagcacactgaagagtgcacatgcgggttcgccagcaataaaaccatagaatgtgcctgtagagacaacagttacacagcaaaaagaccctttgtcaaattaaatgtggaaactgatacagctgaaataagattgatgtgcacaaagacttatctagacactcccagaccggatgatggaagcatagcagggccttgcgaatctaatggagacaagtggcttggaggcatcaaaggaggattcgtccatcaaagaatggcatctaagattggaagatggtactcccgaacgatgtctaaaactaacagaatggggatggaactgtatgtaaagtatgatggtgacccatggactgacagtgatgctcttactcttagtggagtaatggtttccatagaagaacctggttggtattcttttggcttcgaaataaaggacaagaaatgtgatgtcccttgtattgggatagagatggtacacgatggtggaaaagatacttggcattcagctgcaacagccatttactgtttgatgggctcaggacaattgctatgggacactgtcacaggcgttgatatggctttataatagaggaatggttggatctgttctaaaccctttgttcctattttatttgaacagttgttcttactagatttaattgtttctgaaaaatgctcttgttactact

SEQ ID NO: 13 - RNA, 2341 nt, Influenza A virus
  source 1..2341: mol_type = viral cRNA; strain = A/Puerto Rico/8/1934(H1N1)
agcgaaagcaggtcaattatattcaatatggaaagaataaaagaactaagaaatctaatgtcgcagtctcgcacccgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcaaggacaaactttatggagtaaaatgaatgatgccggatcagaccgagtgatggtatcacctctggctgtgacatggtggaataggaatggaccaatgacaaatacagttcattatccaaaaatctacaaaacttattttgaaagagtcgaaaggctaaagcatggaacctttggccctgtccattttagaaaccaagtcaaaatacgtcggagagttgacataaatcctggtcatgcagatctcagtgccaaggaggcacaggatgtaatcatggaagttgttttccctaacgaagtgggagccaggatactaacatcggaatcgcaactaacgataaccaaagagaagaaagaagaactccaggattgcaaaatttctcctttgatggttgcatacatgttggagagagaactggtccgcaaaacgagattcctcccagtggctggtggaacaagcagtgtgtacattgaagtgttgcatttgactcaaggaacatgctgggaacagatgtatactccaggaggggaagtgaagaatgatgatgttgatcaaagcttgattattgctgctaggaacatagtgagaagagctgcagtatcagcagacccactagcatctttattggagatgtgccacagcacacagattggtggaattaggatggtagacatccttaagcagaacccaacagaagagcaagccgtgggtatatgcaaggctgcaatgggactgagaattagctcatccttcagttttggtggattcacatttaagagaacaagcggatcatcagtcaagagagaggaagaggtgcttacgggcaatcttcaaacattgaagataagagtgcatgagggatatgaagagttcacaatggttgggagaagagcaacagccatactcagaaaagcaaccaggagattgattcagctgatagtgagtgggagagacgaacagtcgattgccgaagcaataattgtggccatggtattttcacaagaggattgtatgataaaagcagttagaggtgatctgaatttcgtcaatagggcgaatcagcgactgaatcctatgcatcaacttttaagacattttcagaaggatgcgaaagtgctttttcaaaattggggagttgaacctatcgacaatgtgatgggaatgattgggatattgcccgacatgactccaagcatcgagatgtcaatgagaggagtgagaatcagcaaaatgggtgtagatgagtactccagcacggagagggtagtggtgagcattgaccggttcttgagagtccgggaccaacgaggaaatgtactactgtctcccgaggaggtcagtgaaacacagggaacagagaaactgacaataacttactcatcgtcaatgatgtgggagattaatggtcctgaatcagtgttggtcaatacctatcaatggatcatcagaaactgggaaactgttaaaattcagtggtcccagaaccctacaatgctatacaataaaatggaatttgaaccatttcagtctttagtacctaaggccattagaggccaatacagtgggtttgtgagaactctgttccaacaaatgagggatgtgcttgggacatttgataccgcacagataataaaacttcttcccttcgcagccgctccaccaaagcaaagtagaatgcagttctcctcatttactgtgaatgtgaggggatcaggaatgagaatacttgtaaggggcaattctcctgtattcaactacaacaaggccacgaagagactcacagttctcggaaaggatgctggcactttaaccgaagacccagatgaaggcacagctggagtggagtccgctgttctgaggggattcctcattctgggcaaagaagacaggagatatgggccagcattaagcatcaatgaactgagcaaccttgcgaaaggagagaaggctaatgtgctaattgggcaaggagacgtggtgttggtaatgaaacgaaaacgggactctagcatacttactgacagccagacagcgaccaaaagaattcggatggccatcaattagtgtcgaatagtttaaaaacgaccttgtttctact

SEQ ID NO: 14 - RNA, 2341 nt, Influenza A virus
  source 1..2341: mol_type = viral cRNA; strain = A/Puerto Rico/8/1934(H1N1)
agcgaaagcaggcaaaccatttgaatggatgtcaatccgaccttacttttcttaaaagtgccagcacaaaatgctataagcacaactttcccttataccggagaccctccttacagccatgggacaggaacaggatacaccatggatactgtcaacaggacacatcagtactcagaaaaggcaagatggacaacaaacaccgaaactggagcaccgcaactcaacccgattgatgggccactgccagaagacaatgaaccaagtggttatgcccaaacagattgtgtattggaagcaatggctttccttgaggaatcccatcctggtatttttgaaaactcgtgtattgaaacgatggaggttgttcagcaaacacgagtagacaagctgacacaaggccgacagacctatgactggactttaaatagaaaccagcctgctgcaacagcattggccaacacaatagaagtgttcagatcaaatggcctcacggccaatgagtctggaaggctcatagacttccttaaggatgtaatggagtcaatgaaaaaagaagaaatggggatcacaactcattttcagagaaagagacgggtgagagacaatatgactaagaaaatgataacacagagaacaataggtaaaaggaaacagagattgaacaaaaggagttatctaattagagcattgaccctgaacacaatgaccaaagatgctgagagagggaagctaaaacggagagcaattgcaaccccagggatgcaaataagggggtttgtatactttgttgagacactggcaaggagtatatgtgagaaacttgaacaatcagggttgccagttggaggcaatgagaagaaagcaaagttggcaaatgttgtaaggaagatgatgaccaattctcaggacaccgaactttctttgaccatcactggagataacaccaaatggaacgaaaatcagaatcctcggatgtttttggccatgatcacatatatgaccagaaatcagcccgaatggttcagaaatgttctaagtattgctccaataatgttctcaaacaaaatggcgagactgggaaaagggtatatgtttgagagcaagagtatgaaacttagaactcaaatacctgcagaaatgctagcaagcattgatttgaaatatttcaatgattcaacaagaaagaagattgaaaaaatccgaccgctcttaatagaggggactgcatcattgagccctggaatgatgatgggcatgttcaatatgttaagcactgtattaggcgtctccatcctgaatcttggacaaaagagatacaccaagactacttactggtgggatggtcttcaatcctctgacgattttgctctgattgtgaatgcacccaatcatgaagggattcaagccggagtcgacaggttttatcgaacctgtaagctacatggaatcaatatgagcaagaaaaagtcttacataaacagaacaggtacatttgaattcacaagttttttctatcgttatgggtttgttgccaatttcagcatggagcttcccagttttggtgtgtctgggagcaacgagtcagcggacatgagtattggagttactgtcatcaaaaacaatatgataaacaatgatcttggtccagcaacagctcaaatggcccttcagttgttcatcaaagattacaggtacacgtaccgatgccatagaggtgacacacaaatacaaacccgaagatcatttgaaataaagaaactgtgggagcaaacccgttccaaagctggactgctggtctccgacggaggcccaaatttatacaacattagaaatctccacattcctgaagtctgcctaaaatgggaattgatggatgaggattaccaggggcgtttatgcaacccactgaacccatttgtcagccataaagaaattgaatcaatgaacaatgcagtgatgatgccagcacatggtccagccaaaaacatggagtatgatgctgttgcaacaacacactcctggatccccaaaagaaatcgatccatcttgaatacaagtcaaagaggagtacttgaagatgaacaaatgtaccaaaggtgctgcaatttatttgaaaaattcttccccagcagttcatacagaagaccagtcgggatatccagtatggtggaggctatggtttccagagcccgaattgatgcacggattgatttcgaatctggaaggataaagaaagaagagttcactgagatcatgaagatctgttccaccattgaagagctcagacggcaaaaatagtgaatttagcttgtccttcatgaaaaaatgccttgttcctact

SEQ ID NO: 15 - RNA, 2233 nt, Influenza A virus
  source 1..2233: mol_type = viral cRNA; strain = A/Puerto Rico/8/1934(H1N1)
agcgaaagcaggtactgatccaaaatggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagatttccacttcatcaatgagcaaggcgagtcaataatcgtagaacttggtgatcctaatgcacttttgaagcacagatttgaaataatcgagggaagagatcgcacaatggcctggacagtagtaaacagtatttgcaacactacaggggctgagaaaccaaagtttctaccagatttgtatgattacaaggaaaatagattcatcgaaattggagtaacaaggagagaagttcacatatactatctggaaaaggccaataaaattaaatctgagaaaacacacatccacattttctcgttcactggggaagaaatggccacaaaggccgactacactctcgatgaagaaagcagggctaggatcaaaaccaggctattcaccataagacaagaaatggccagcagaggcctctgggattcctttcgtcagtccgagagaggagaagagacaattgaagaaaggtttgaaatcacaggaacaatgcgcaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttttgaaaacaacaccacgaccacttagacttccgaatgggcctccctgttctcagcggtccaaattcctgctgatggatgccttaaaattaagcattgaggacccaagtcatgaaggagagggaataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaagactaaaaatatgaaaaaaacaagtcagctaaagtgggcacttggtgagaacatggcaccagaaaaggtagactttgacgactgtaaagatgtaggtgatttgaagcaatatgatagtgatgaaccagaattgaggtcgcttgcaagttggattcagaatgagttcaacaaggcatgcgaactgacagattcaagctggatagagcttgatgagattggagaagatgtggctccaattgaacacattgcaagcatgagaaggaattatttcacatcagaggtgtctcactgcagagccacagaatacataatgaagggggtgtacatcaatactgccttacttaatgcatcttgtgcagcaatggatgatttccaattaattccaatgataagcaagtgtagaactaaggagggaaggcgaaagaccaacttgtatggtttcatcataaaaggaagatcccacttaaggaatgacaccgacgtggtaaactttgtgagcatggagttttctctcactgacccaagacttgaaccacacaaatgggagaagtactgtgttcttgagataggagatatgcttctaagaagtgccataggccaggtttcaaggcccatgttcttgtatgtgaggacaaatggaacctcaaaaattaaaatgaaatggggaatggagatgaggcgttgtctcctccagtcacttcaacaaattgagagtatgattgaagctgagtcctctgtcaaagagaaagacatgaccaaagagttctttgagaacaaatcagaaacatggcccattggagagtctcccaaaggagtggaggaaagttccattgggaaggtctgcaggactttattagcaaagtcggtatttaacagcttgtatgcatctccacaactagaaggattttcagctgaatcaagaaaactgcttcttatcgttcaggctcttagggacaatctggaacctgggacctttgatcttggggggctatatgaagcaattgaggagtgcctaattaatgatccctgggttttgcttaatgcttcttggttcaactccttccttacacatgcattgagttagttgtggcagtgctactatttgctatccatactgtccaaaaaagtaccttgtttctact

SEQ ID NO: 16 - RNA, 1565 nt, Influenza A virus
  source 1..1565: mol_type = viral cRNA; strain = A/Puerto Rico/8/1934(H1N1)
agcaaaagcagggtagataatcactcactgagtgacatcaaaatcatggcgtcccaaggcaccaaacggtcttacgaacagatggagactgatggagaacgccagaatgccactgaaatcagagcatccgtcggaaaaatgattggtggaattggacgattctacatccaaatgtgcacagaacttaaactcagtgattatgagggacggttgatccaaaacagcttaacaatagagagaatggtgctctctgcttttgacgaaaggagaaataaatacctggaagaacatcccagtgcggggaaggatcctaagaaaactggaggacctatatacagaagagtaaacggaaagtggatgagagaactcatcctttatgacaaagaagaaataaggcgaatctggcgccaagctaataatggtgacgatgcaacggctggtctgactcacatgatgatctggcattccaatttgaatgatgcaacttatcagaggacaagggctcttgttcgcaccggaatggatcccaggatgtgctctctgatgcaaggttcaactctccctaggaggtctggagccgcaggtgctgcagtcaaaggagttggaacaatggtgatggaattggtcaggatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccgggacccagggaatgctgagttcgaagatctcacttttctagcacggtctgcactcatattgagagggtcggttgctcacaagtcctgcctgcctgcctgtgtgtatggacctgccgtagccagtgggtacgactttgaaagagagggatactctctagtcggaatagaccctttcagactgcttcaaaacagccaagtgtacagcctaatcagaccaaatgagaatccagcacacaagagtcaactggtgtggatggcatgccattctgccgcatttgaagatctaagagtattgagcttcatcaaagggacgaaggtggtcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccgttatggcagcattcactgggaatacagaggggagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

SEQ ID NO: 17 - RNA, 1027 nt, Influenza A virus
  source 1..1027: mol_type = viral cRNA; strain = A/Puerto Rico/8/1934(H1N1)
agcgaaagcaggtagatattgaaagatgagtcttctaaccgaggtcgaaacgtacgttctctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaacccactaatcagacatgagaacagaatggttttagccagcactacagctaaggctatggagcaaatggctggatcgagtgagcaagcagcagaggccatggaggttgctagtcaggctaggcaaatggtgcaagcgatgagaaccattgggactcatcctagctccagtgctggtctgaaaaatgatcttcttgaaaatttgcaggcctatcagaaacgaatgggggtgcagatgcaacggttcaagtgatcctctcgctattgccgcaaatatcattgggatcttgcacttgatattgtggattcttgatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaaaaaactaccttgtttctact

SEQ ID NO: 18 - RNA, 890 nt, Influenza A virus
  source 1..890: mol_type = viral cRNA; strain = A/Puerto Rico/8/1934(H1N1)
agcaaaagcagggtgacaaagacataatggatccaaacactgtgtcaagctttcaggtagattgctttctttggcatgtccgcaaacgagttgcagaccaagaactaggtgatgccccattccttgatcggcttcgccgagatcagaaatccctaagaggaaggggcagcactcttggtctggacatcgagacagccacacgtgctggaaagcagatagtggagcggattctgaaagaagaatccgatgaggcacttaaaatgaccatggcctctgtacctgcgtcgcgttacctaaccgacatgactcttgaggaaatgtcaagggaatggtccatgctcatacccaagcagaaagtggcaggccctctttgtatcagaatggaccaggcgatcatggataaaaacatcatactgaaagcgaacttcagtgtgatttttgaccggctggagactctaatattgctaagggctttcaccgaagagggagcaattgttggcgaaatttcaccattgccttctcttccaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaaggtaacagagaatagttttgagcaaataacatttatgcaagccttacatctattgcttgaagtggagcaagagataagaactttctcatttcagcttatttaataataaaaaacacccttgtttctact

SEQ ID NO: 19 - RNA, 2341 nt, Influenza A virus
  source 1..2341: mol_type = viral cRNA; strain = A/New York/392/2004(H3N2)
agcaaaagcaggtcaattatattcagtatggaaagaataaaagaactacggaacctgatgtcgcagtctcgcactcgcgagatactgacaaaaaccacagtggaccatatggccataattaagaagtacacatcggggagacaggaaaagaacccgtcacttaggatgaaatggatgatggcaatgaaatacccaatcactgctgacaaaaggataacagaaatggttccggagagaaatgaacaaggacaaactctatggagtaaaatgagtgatgctggatcagatcgagtgatggtatcacctttggctgtaacatggtggaatagaaatggacccgtggcaagtacggtccattacccaaaagtatacaagacttattttgacaaagtcgaaaggttaaaacatggaacctttggccctgttcattttagaaatcaagtcaagatacgcagaagagtagacataaaccctggtcatgcagacctcagtgccaaagaggcacaagatgtaattatggaagttgtttttcccaatgaagtgggagccaggatactaacatcagaatcgcaattaacaataactaaagagaaaaaagaagaactccgagattgcaaaatttctcccttgatggttgcatacatgttagagagagaacttgtccgaaaaacaagatttctcccagttgctggcggaacaagcagtatatacattgaagtcttacatttgactcaaggaacgtgttgggaacaaatgtacactccaggtggagaagtgaggaatgacgatgttgaccaaagcctaattattgcggccaggaacatagtaagaagagctgcagtatcagcagatccactagcatctttattggagatgtgccacagcacacaaattggcgggacaaggatggtggacattcttagacagaacccgactgaagaacaagctgtggatatatgcaaggctgcaatgggattgagaatcagctcatccttcagctttggtgggtttacatttaaaagaacaagcgggtcatcagtcaaaaaagaggaagaagtgcttacaggcaatctccaaacattgaagataagagtacatgaggggtatgaggagttcacaatggtggggaaaagagcaacagctatactcagaaaagcaaccagaagattggttcagctcatagtgagtggaagagacgaacagtcaatagccgaagcaataatcgtggccatggtgttttcacaagaggattgcatgataaaagcagttagaggtgacctgaatttcgtcaacagagcaaatcaacggttgaaccccatgcatcagcttttaaggcattttcagaaagatgcgaaagtgctttttcaaaattggggaattgaacacatcgacagtgtgatgggaatggttggagtattaccagatatgactccaagcacagagatgtcaatgagaggaataagagtcagcaaaatgggtgtggatgaatactccagtacagagagggtggtggttagcattgatcggtttttgagagttcgagaccaacgcgggaatgtattattgtctcctgaggaggtcagtgaaacacagggaactgaaagattgacaataacatattcatcgtcgatgatgtgggagattaacggtcctgagtcggttttggtcaatacctatcaatggatcatcagaaattgggaagctgtcaaaattcaatggtctcagaatcctgcaatgttgtacaacaaaatggaatttgaaccatttcaatctttagtccccaaggccattagaagccaatacagtgggtttgtcagaactctattccaacaaatgagagacgtacttgggacatttgacaccacccagataataaagcttctcccttttgcagccgctccaccaaagcaaagcagaatgcagttctcttcactgactgtaaatgtgaggggatcagggatgagaatacttgtaaggggcaattctcctgtattcaactacaacaagaccactaaaagactaacaattctcggaaaagatgccggcactttaattgaagacccagatgaaagcacatccggagtggagtccgccgtcttgagagggtttctcattataggtaaggaagacagaagatacggaccagcattaagcatcaatgaactgagtaaccttgcaaaaggggaaaaggctaatgtgctaatcgggcaaggagacgtggtgttggtaatgaaacgaaaacgggactctagcatacttactgacagccagacagcgaccaaaagaattcggatggccatcaattaatgttgaatagtttaaaaacgaccttgtttctact

SEQ ID NO: 20 - RNA, 2341 nt, Influenza A virus
  source 1..2341: mol_type = viral cRNA; strain = A/New York/392/2004(H3N2)
agcaaaagcaggcaaaccatttgaatggatgtcaatccgactctactgttcctaaaggttccagcgcaaaatgccataagcaccacattcccttatactggagatcctccatacagccatggaacaggaacaggatacaccatggacacagtcaacagaacacaccaatattcagagaaggggaagtggacgacaaatacagaaactggggcaccccaactcaacccaattgatggaccactacctgaggataatgagccaagtggatatgcacaaacagactgtgtcctggaggctatggccttccttgaagaatcccacccaggtatctttgagaactcatgccttgaaacaatggaagtcgttcaacaaacaagggtggacaaactaacccaaggccgccagacttatgattggacattaaacagaaatcaaccggcagcaactgcattagccaacaccatagaagtttttagatcgaatggactaacagccaatgaatcaggaaggctaatagatttcctcaaggatgtgatggaatcaatggataaagaggaaatggagataacaacacactttcaaagaaaaaggagagtaagagacaacatgaccaagaaaatggtcacacaaagaacaatagggaagaaaaaacaaagagtgaataagagaggctatctaataagagctttgacattgaacacgatgaccaaagatgcagagagaggtaaattaaaaagaagggctattgcaacacccgggatgcaaattagagggttcgtgtacttcgttgaaactttagctagaagcatttgcgaaaagcttgaacagtctggacttccggttgggggtaatgaaaagaaggccaaactggcaaatgttgtgagaaaaatgatgactaattcacaagacactgagctttctttcacaatcactggggacaacactaagtggaatgaaaatcaaaaccctcgaatgtttttggcgatgattacatatatcacaaaaaatcaacctgagtggttcagaaacatcctgagcatcgcaccaataatgttctcaaacaaaatggcaagactaggaaaaggatacatgttcgagagtaagagaatgaagctccgaacacaaatacccgcagaaatgctagcaagcattgacctgaagtatttcaatgaatcaacaaggaagaaaattgagaaaataaggcctcttctaatagatggcacagcatcattgagccctgggatgatgatgggcatgttcaacatgctaagtacggttttaggagtctcggtactgaatcttgggcaaaagaaatacaccaagacaacatactggtgggatgggctccaatcctccgacgattttgccctcatagtgaatgcaccaaatcatgagggaatacaagcaggagtggatagattctacaggacctgcaagttagtgggaatcaacatgagcaaaaagaagtcctatataaataaaacagggacatttgaattcacaagctttttttatcgatatggatttgtggctaattttagcatggagcttcccagttttggagtgtctggaataaacgagtcagctgatatgagtattggagtaacagtgataaagaacaacatgataaacaatgaccttgggccagcaacagcccagatggctctccaattgttcatcaaagactacagatatacatataggtgccatagaggagacacacaaattcagacgagaagatcattcgagctaaagaagctgtgggatcaaacccaatcaagggcaggactattggtatcagatgggggaccaaacttatacaatatccggaaccttcacatccctgaagtctgcttaaagtgggagctaatggatgagaattatcggggaagactttgtaaccccctgaatccctttgtcagccataaagaaattgagtctgtaaacaatgctgtagtgatgccagcccacggtccagccaaaagtatggaatatgatgccgttgcaactacacactcctggaatcccaagaggaaccgctctattctaaacactagccaaaggggaattcttgaggatgaacagatgtaccaaaagtgctgcaacttgttcgagaaatttttccctagtagttcatataggagaccgattggaatttctagcatggtggaggccatggtgtctagggcccggattgatgccagaattgacttcgagtctggacggattaagaaggaagagttctctgagatcatgaagatctgttccaccattgaagaactcagacggcaaaaataatgaatttagcttgtccttcatgaaaaaatgccttgtttctact

SEQ ID NO: 21 - RNA, 2233 nt, Influenza A virus
  source 1..2233: mol_type = viral cRNA; strain = A/New York/392/2004(H3N2)
agcaaaagcaggtactgattcgaaatggaagattttgtgcgacaatgcttcaacccgatgattgtcgaacttgcagaaaaagcaatgaaagagtatggagaggatctgaaaattgaaacaaacaaatttgcagcaatatgcacccacttggaggtatgtttcatgtattcagattttcatttcatcaatgaacaaggcgaatcaatagtggtagaacttgatgatccaaatgcactgttaaagcacagatttgaaataatcgaggggagagacagaacaatggcctggacagtagtaaacagtatctgcaacactactggagcagaaaaaccaaagtttctaccagatttgtatgattacaaggagaatagattcatcgaaattggagtgacaagaagagaagtccacatatattaccttgaaaaggccaataaaattaaatctgagaacacacacattcacatcttctcattcactggggaggaaatagccacaaaggcagactacactctcgacgaggaaagcagggctaggattaaaaccaggctatttaccataagacaagaaatggccaacagaggcctctgggattcctttcgtcagtccgaaagaggcgaagaaacaattgaagaaaaatttgaaatctcaggaactatgcgtaggcttgccgaccaaagtctcccaccgaaattctcctgccttgagaattttagagcctatgtggatggattcgaaccgaacggctgcattgagggcaagctttctcaaatgtccaaagaagtgaatgccaaaattgaaccttttctgaagacaacaccaagaccaatcaaacttcctaatggacctccttgttatcagcggtccaaattcctcctgatggatgctttgaaattgagcattgaagacccaagtcatgaaggagaagggattccattatatgatgcgatcaagtgcataaaaacattctttggatggaaagaaccttatatagtcaaaccacacgaaaagggaataaattcaaattacctgctgtcatggaagcaagtattgtcagaattgcaggacattgaaaatgaggagaagatcccaaggactaaaaacatgaagaaaacgagtcaactaaagtgggctcttggtgaaaacatggcaccagagaaagtagactttgacaactgcagagacataagcgatttgaagcaatatgatagtgacgaacctgaattaaggtcactttcaagctggatacagaatgagttcaacaaggcctgcgagctaactgattcaatctggatagagctcgatgaaattggagaggacgtagccccaattgagtacattgcaagcatgaggaggaattatttcacagcagaggtgtcccattgtagagccactgagtacataatgaagggggtatacattaatactgccctgctcaatgcatcctgtgcagcaatggacgattttcaactaattcccatgataagcaagtgcagaactaaagagggaaggcgaaaaaccaatttatatggattcatcataaagggaagatctcatttaaggaatgacacagatgtggtaaactttgtgagcatggagttttctctcactgacccgagacttgagccacataaatgggagaaatactgtgtccttgagataggagatatgttactaagaagtgccataggccaaatttcaaggcctatgttcttgtatgtgaggacaaacggaacatcaaaggtcaaaatgaaatggggaatggagatgagacgttgcctccttcagtcactccagcagatcgagagcatgattgaagccgagtcctcgattaaagagaaagacatgaccaaagagttttttgagaataaatcagaagcatggcccattggggagtcccccaagggagtggaagaaggttccattgggaaagtctgtaggactctattggctaagtcagtgttcaatagcctgtatgcatcaccacaattggaaggattttcagcggagtcaagaaaactgcttcttgttgttcaggctcttagggacaacctcgaacctgggacctttgatctcggggggctatatgaagcaattgaggagtgcctgattaatgatccctgggttttgctcaatgcatcttggttcaactccttcctgacacatgcattaaaatagttatggcagtgctactatttgttatccgtactgtccaaaaaagtaccttgtttctact

SEQ ID NO: 22 - RNA, 1566 nt, Influenza A virus
  source 1..1566: mol_type = viral cRNA; strain = A/New York/392/2004(H3N2)
agcaaaagcagggttaataatcactcaccgagtgacatcaaaatcatggcgtcccaaggcaccaaacggtcttatgaacagatggaaactgatggggatcgccagaatgcaactgagattagggcatccgtcgggaagatgattgatggaattgggagattctacatccaaatgtgcactgaacttaaactcagtgatcatgaagggcggttgatccagaacagcttgacaatagagaaaatggtgctctctgcttttgatgaaagaaggaataaatacctggaagaacaccccagcgcggggaaagatcccaagaaaactggggggcccatatacaggagagtagatggaaaatggatgagggaactcgtcctttatgacaaagaagagataaggcgaatctggcgccaagccaacaatggtgaggatgcgacagctggtctaactcacataatgatctggcattccaatttgaatgatgcaacataccagaggacaagagctcttgttcgaactggaatggatcccagaatgtgctctctgatgcagggctcgactctccctagaaggtccggagctgcaggtgctgcagtcaaaggaatcgggacaatggtgatggaactgatcagaatggtcaaacgggggatcaacgatcgaaatttctggagaggtgagaatgggcggaaaacaagaagtgcttatgagagaatgtgcaacattcttaaaggaaaatttcaaacagctgcacaaagagcaatggtggatcaagtgagagaaagtcggaacccaggaaatgctgagatcgaagatctcatatttttggcaagatctgcattgatattgagagggtcagttgctcacaaatcttgcctacctgcctgtgcgtatggacctgcagtatccagtgggtacgacttcgaaaaagagggatattccttggtgggaatagaccctttcaaactacttcaaaatagccaaatatacagcctaatcagacctaacgagaatccagcacacaagagtcagctggtgtggatggcatgccattctgctgcatttgaagatttaagattgttaagcttcatcagagggacaaaagtatctccgcgggggaaactgtcaactagaggagtacaaattgcttcaaatgagaacatggataatatgggatcgagcactcttgaactgagaagcgggtactgggccataaggaccaggagtggaggaaacactaatcaacagagggcctccgcaggccaaaccagtgtgcaacctacgttttctgtacaaagaaacctcccatttgaaaagtcaaccatcatggcagcattcactggaaatacggagggaaggacttcagacatgagggcagaaatcataagaatgatggaaggtgcaaaaccagaagaagtgtcattccgggggaggggagttttcgagctctcagacgagaaggcaacgaacccgatcgtgccctcttttgatatgagtaatgaaggatcttatttcttcggagacaatgcagaagagtacgacaattaaggaaaaaatacccttgtttctact

SEQ ID NO: 23 - RNA, 1027 nt, Influenza A virus
  source 1..1027: mol_type = viral cRNA; strain = A/New York/392/2004(H3N2)
agcaaaagcaggtagatattgaaagatgagccttctaaccgaggtcgaaacgtatgttctctctatcgttccatcaggccccctcaaagccgagatcgcgcagagacttgaagatgtctttgctgggaaaaacacagatcttgaggctctcatggaatggctaaagacaagaccaattctgtcacctctgactaaggggattttggggtttgtgttcacgctcaccgtgcccagtgagcgaggactgcagcgtagacgctttgtccaaaatgccctcaatgggaatggagatccaaataacatggacaaagcagttaaactgtataggaaacttaagagggagataacgttccatggggccaaagaaatagctctcagttattctgctggtgcacttgccagttgcatgggcctcatatacaataggatgggggctgtaaccactgaagtggcatttggcctggtatgtgcaacatgtgaacagattgctgactcccagcacaggtctcataggcaaatggtggcaacaaccaatccattaataaaacatgagaacagaatggttttggccagcactacagctaaggctatggagcaaatggctggatcaagtgagcaggcagcggaggccatggaaattgctagtcaggccaggcaaatggtgcaggcaatgagagccgttgggactcatcctagctccagtactggtctaagagatgatcttcttgaaaatttgcagacctatcagaaacgaatgggggtgcagatgcaacgattcaagtgacccgcttgttgttgccgcgagtatcattgggatcttgcacttgatattgtggattcttgatcgtctttttttcaaatgcgtctatcgactcttcaaacacggccttaaaagaggcccttctacggaaggagtacctgagtctatgagggaagaatatcgaaaggaacagcagaatgctgtggatgctgacgacagtcattttgtcagcatagagttggagtaaaaaactaccttgtttctact

SEQ ID NO: 24 - RNA, 890 nt, Influenza A virus
  source 1..890: mol_type = viral cRNA; strain = A/New York/392/2004(H3N2)
agcaaaagcagggtgacaaagacataatggattccaacactgtgtcaagtttccaggtagattgctttctttggcatatccggaaacaagttgtagaccaagaactgagtgatgccccattccttgatcggcttcgccgagatcagaggtccctaaggggaagaggcaatactctcggtctagacatcaaagcagccacccatgttggaaagcaaattgtagaaaagattctgaaagaagaatctgatgaggcacttaaaatgaccatggtctccacacctgcttcgcgatacataactgacatgactattgaggaattgtcaagaaactggttcatgctaatgcccaagcagaaagtggaaggacctctttgcatcagaatggaccaggcaatcatggagaaaaacatcatgttgaaagcgaatttcagtgtgatttttgaccgactagagaccatagtattactaagggctttcaccgaagagggagcaattgttggcgaaatctcaccattgccttcttttccaggacatactattgaggatgtcaaaaatgcaattggggtcctcatcggaggacttgaatggaatgataacacagttcgagtctctaaaaatctacagagattcgcttggagaagcagtaatgagaatgggggacctccacttactccaaaacagaaacggaaaatggcgagaacagctaggtcaaaagtttgaagagataagatggctgattgaagaagtgagacacagactaaaaacaactgaaaatagctttgaacaaataacattcatgcaagcattacaactgctgtttgaagtggaacaggagataagaactttctcatttcagcttatttaatgataaaaaacacccttgtttctact

SEQ ID NO: 25 - RNA, 2341 nt, Influenza A virus
  source 1..2341: mol_type = viral cRNA; strain = A/Goose/Guangdong/1/96(H5N1)
agcaaaagcaggtcaattatattcaatatggaaagaataaaagaactaagagatctaatgtcgcagtcccgcactcgcgagatactaacaaaaaccactgtggatcatatggccataatcaagaaatacacatcaggaagacaagagaagaaccctgctctcagaatgaaatggatgatggcaatgaaatatccaatcacagcagacaagagaataatggagatgattcctgaaaggaatgagcaaggacaaacgctttggagcaagacaaatgatgctgggtcggacagagtgatggtgtctcccctagctgtaacttggtggaacaggaatgggccgacaacaagtacagtccattatccaaaggtttacaaaacatactttgagaaggttgaaaggttaaaacatggaaccttcggtcccgttcatttccgaaaccaagttaaaatacgtcgccgggtggatataaacccgggccatgcagatctcagtgctaaagaagcacaagatgttatcatggaggtcgttttcccaaatgaagtgggagctagaatattgacatcagagtcgcaattgacaataacaaaagagaagaaagaagagctccaggattgtaaaattgctcctttaatggtggcatacatgttggaaagagaactggtccgcaaaaccagatttctaccggtagcaggcggaacaagcagtgtgtacattgaggtattgcatttgactcaagggacctgttgggaacagatgtacactcccggcggagaagtaagaaatgatgatgttgaccagagtttgatcatcgctgccagaaacattgttaggagagcaacagtatcagcggacccactggcatcactcttggagatgtgtcacagcacacaaattgggggaataaggatggtggacatccttaggcaaaacccaactgaggagcaagctgtggatatatgcaaagcagcaatgggtttgaggatcagttcatcctttagctttggaggcttcactttcaaaagaacaaatggatcatccgtcaagaaggaagaggaagtgcttacaggcaacctccaaacattgaaaataaaagtacatgaggggtatgaagaattcacaatggttgggcggagagcaacagctatcctgaggaaagcaactagaaggctgattcagttgatagtaagtggaagagatgaacaatcaatcgctgaagcgatcattgtagcaatggtgttctcacaggaggattgcatgataaaggcagtccgaggcgatctgaatttcgtgaacagagcaaaccaaagattgaaccccatgcatcaactcctgaggcacttccaaaaagatgcaaaagtgctgtttcagaactggggaattgaacctattgacaatgtcatggggatgatcggaatattacctgacatgactccaagcgcagagatgtcactgagaggagtgagagttagtaagatgggagtagatgaatattccagcacggagagagtggtggtgagtattgaccgtttcttgagggtccgagatcagcaggggaacgtactcttatctcctgaagaggttagtgaaacacagggaacagagaagttgacaataacatattcatcctcaatgatgtgggaaatcaacggtcctgagtcagtgcttgttaacacttatcaatggatcatcaggaattgggagactgtaaagattcaatggtctcaagatcccacaatgctgtacaataagatggagtttgaatcgttccaatccttggtgccaaaggctgccagaagccaatatagtggatttgtgagaacactattccaacagatgcgtgatgttttggggacatttgatactgtccaaataatcaagctgctaccatttgcagcagccccaccggagccgagcagaatgcagttttcttctctaactgtgaatgtgagaggctcaggaatgagaatactcgtgaggggtaactcccccgtgttcaactacaacaaggcaaccaaaaggcttacagtcctcggaaaggacgcaggtgcattaacagaagatccagacgagggaacagccggggtggaatctgcagtattgaggggattcctaattctaggcagagaggacaaaagatatggacccgcattgagcatcaatgaactgagcaatcttgcaaaaggggagaaggctaatgtattgataatgcaaggagacgtggtgttggtaatgaaacggaaacgggactttagcatacttactgacagccagacagcgaccaaaagaattcggatggccatcaattagtgttgaatagtttaaaaacgaccttgtttctact

SEQ ID NO: 26 - RNA, 2341 nt, Influenza A virus
  source 1..2341: mol_type = viral cRNA; strain = A/goose/Guangdong/1/1996(H5N1)
agcaaaagcaggcaaaccatttgaatggatgtcaatccgactttacttttcttaaaagtgccagcgcaaaatgctataagtaccacattcccttatactggagatcctccatacagccatggaacaggaacaggatacaccatggacacagtcaacagaacacatcaatattcagaaaaggggaaatggacaacgaacacagagactggagcaccccaactcaatccgattgatggaccactacctgaggataatgagccgagtgggtatgcacaaacagattgtgtattggaagcaatggctttccttgaagaatcccacccagggatctttgaaaactcgtgtcttgaaacgatggaagttgttcagcaaacaagagtggataagctgacccaaggtcgccaaacctatgactggacattgaaaagaaaccagccggctgcaaccgctttggccaacactatagaggtcttcagatcgaatggtctaacagccaatgaatcgggaaggctaatagatttcctcaaagacgtgatggaatcaatggataagggagaaatggaaataataacacatttccagagaaagagaagagtgagggacaacatgaccaagaaaatggtcacacaaagaacaatagggaagaaaaaacaaaggctgaacaaaaggagctacctaataagagcactgacactgaacacaatgacaaaagacgcagaaagaggcaaattgaagaggcgggcaattgcaacacccgggatgcaaatcagaggattcgtgtactttgtcgaaacactagcgaggagtatctgtgagaaacttgagcaatctggactccccgtcggagggaatgaaaagaaggctaaattggcaaatgtcgtgaggaagatgatgactaactcacaagatacagagctctcttttacaattactggagacaacaccaaatggaatgagaatcagaaccctcggatgtttctagcaatgataacatacatcacaaggaaccaacctgaatggtttagaaatgtcttaagcattgctcctataatgttctcaaacaagatggcaagattagggaaaggatacatgttcgaaagtaagagcatgaagctacggacacaaataccagcagaaatgcttgcaagcattgacttgaaatacttcaacgaatcaacgagaaagaaaatcgagaaaataagacctctactaatagatggcacagcctcattgagtcctggaatgatgatgggcatgttcaatatgctgagtacagtcttaggagtttcaatcctgaatcttgggcagaagaggtacaccaaaaccacatactggtgggacggactccaatcctctgatgatttcgctctcatagtgaatgcaccaaatcatgagggaatagaagcaggggtggataggttctataggacttgcaaactagttggaatcaatatgaccaagaagaagtcttacataaatcggacaggaacatgtgaattcacaagcttcttctaccgctatgggttcgtagccaacttcagtatggagctgcccagctttggagtgtctgggattaatgaatcggctgacatgagcattggtgttacagtgataaagaacaatatgatggacaacgaccttggaccagcaacagctcagatggctcttcagctattcattaaggactacagatacccataccgatgccacaggggggatacacaaatccaaacgaggagatcattcgagctgaagaagctgtgggagcagacccgctcaaaggcaggactgttggtttcagatggaggaccaaacccatacaatatccggaatctccacattccggaggctggcttgaagtgggaattgatggatgaagactaccagggcagactgtgtaatcctctgaacccgtttgttagtcataaggaaattgagtctgtcaacaatgctgtggtaatgccagctcatggcccagccaagagcatggaatatgatgcagttgcgactacacattcatggattcccaagaggaatcgttccattctcaacaccagccaaagggggattcttgaggatgaacagatgtatcagaagtgctgcaatctattcgagaaattcttccctagcagttcatatcggaggccagttggaatttccagcatggtggaggccatggtgtctagggcccgaattgatgcacgaattgacttcgagtctggaaggattaagaaagaagagtttgctgagatcatgaagatctgttccaccattgaagagctcggacggcaaaaatagtgaatttagcttgtccttcatgaaaaaatgccttgtttctact

SEQ ID NO: 27 - RNA, 2233 nt, Influenza A virus
  source 1..2233: mol_type = viral cRNA; strain = A/goose/Guangdong/1/1996(H5N1)
agcaaaagcaggtactgatccaaaatggaagactttgtgcgacaatgcttcaatccaatgattgtcgagcttgcggaaaaggcaatgaaagaatatggggaagatccgaaaatcgaaacgaacaaatttgccgcaatatgcacgcacttagaagtctgtttcatgtattcagatttccactttattgatgaacggggcgaatcaacaattatagaatctggcgatcccaatgcattattgaaacaccggtttgaaataatcgaagggagggaccgaacaatggcctggacagtggtgaatagtatctgcaacaccacaggagttgagaagcctaaatttctcccagatttgtatgactacaaggagaaccgatttattgaaattggagtgacacggagggaagttcacacatactatctagaaaaagccaacaagataaaatctgagaagacacacattcacatattctcattcactggagaggaaatggccaccaaagcggactacacccttgatgaagaaagcagggcccgaatcaaaaccaggctgttcactataaggcaggaaatggccagtaggggtttatgggattcctttcgtcagtccgagagaggcgaagagacagttgaagaaagatttgaaatcacagggactatgtgcaggcttgccgaccaaagtctcccacctaatttctccagccttgaaaaatttagagcctatgtggatggattcgaaccgaacggctgcattgagggcaagctttctcaaatgtcgaaagaagtaaacgccagaattgagccatttctgaagacaacaccacgccctcttagattacctgatgggcctccctgctctcagcggtcgaagtttttgctgatggatgcccttaaattaagcatcgaagacccgagtcatgagggggaggggataccgctatatgatgcaatcaaatgcatgaaaacatttttcggctggaaagagcccaacattgtaaaaccacatgaaaaaggcataaaccccaattacctcctggcttggaagcaggtgctggcagagctccaagatattgaaaacgaggagaaaattccaaagacaaagaacatgaggaaaacaagccaattgaagtgggcacttggtgagaatatggcaccagagaaagtagactttgaggattgcaaagatgttagcgatctaaggcagtatgacagtgatgaaccaaagcctagatcactagcaagctggatccagagtgaattcaacaaggcatgcgaattgacagattcaagttggattgaacttgatgaaataggggaagacgttgctccaattgagcacattgcaagtatgagaaggaactatttcacagcggaagtatcccattgcagggctactgaatacataatgaagggagtgtacataaacacagctttgttgaatgcatcctgtgcagccatggatgacttccaactgatcccaatgataagcaaatgcagaaccaaagaaggaagacggaaaactaacctgtatggattccttataaaaggaagatcccatttgagaaatgacaccgatgtggtaaactttgtgagtatggaattctctcttactgatccgaggctggagccacacagatgggaaaagtactgcgttcttcggataggagacatgctcttacggactgaaataggccaagtgtcaaggcccatgtttctttatgtgagaaccaatggaacctccaagatcaagatgaaatggggcatggaaatgaggcgatgcccttttcaatcccttcaacagattgagagcatgattgaggccgagtcttctgtcaaagaaaaagacatgactaaagaattctttgaaaacaaatcagaaacatggccaattggagaatcacccaagggagtggaggaaggctccatcgggaaggtgtgcagaaccttactggctaaatctgttttcaacagtctatatgcatctccacaactcgaggggttttcagctgaatcaagaaaattgcttctcattgttcaggcacttagggacaacctggaacctggaaccttcgatcttggggggctatatgaagcaattgaggagtgcctgattaatgatccctgggttttgcttaatgcatcttggttcaactccttcctcacacatgcactaagatagttgtggcaatgctactatttgctatccatactgtccaaaaaagtaccttgtttctact

SEQ ID NO: 28 - RNA, 1565 nt, Influenza A virus
  source 1..1565: mol_type = viral cRNA; strain = A/Goose/Guangdong/1/96(H5N1)
agcaaaagcagggtagataatcactcactgagtgacatcaacatcatggcgtctcagggcaccaaacgatcttatgaacagatggaaactggtggagaacgccagaatgctactgagatcagagcatctgttggaagaatggttggtggaattgggaggttttatatacagatgtgcactgaactcaaactcagcgactatgaaggaaggctgattcagaacagcataacaatagagagaatggttctctctgcatttgatgaaaggaggaacaaatacctggaagaacatcccagtgcggggaaggacccaaagaaaactggaggtccaatctaccgaagaagagacggaaaatgggtgagagagctgattctgtatgacaaagaggagatcaggagaatttggcgtcaagcgaacaatggagaagatgcaactgctggtctcactcacatgatgatctggcattccaatctaaatgatgccacataccagagaacaagagctctcgtgcgtactgggatggaccctagaatgtgctctctgatgcaaggatcaactctcccgaggagatctggagctgctggtgcggcagtaaagggagtcggaacgatggtgatggaactaattcggatgataaagcgagggattaacgatcggaatttctggagaggtgaaaatgggcgaagaacaagaattgcatatgagagaatgtgcaacatcctcaaagggaaattccaaacagcagcacaaagagcaatgatggatcaggtacgggaaagcagaaatcctgggaatgctgagattgaagatctcatatttctggcacggtctgcactcatcctgagaggatcagtggcccacaagtcctgcttgcctgcttgtgtgtacgggcttgccgtggccagtggatatgactttgagagagaagggtactctctggtcgggattgatcctttccgtctgctgcaaaacagccaggtctttagtctaattagaccaaatgagaatccagcacataaaagtcaattggtgtggatggcatgccattctgcagcatttgaagatctgagagtctcaagcttcatcagagggacaagagtggccccaaggggacaactatctactagaggagttcaaattgcttcaaatgagaacatggaaacaatggactccagcactcttgaactgagaagcagatattgggctataaggaccaggagtggaggaaacaccaaccagcagagagcatctgcaggacaaatcagtgtgcagcctactttctcggtacagagaaatcttcccttcgaaagagcgaccattatggcggcattcacagggaatacagagggcagaacatctgacatgaggactgaaatcataaggatgatggaaagctccagaccagaagatgtgtctttccaggggcggggagtcttcgagctctcggacgaaaaggcaacgaacccgatcgtgccttcctttgacatgagtaatgaaggatcttatttcttcggagacaatgcagaggaatatgacaattgaagaaaaatacccttgtttctact

SEQ ID NO: 29 - RNA, 966 nt, Influenza A virus
  source 1..966: mol_type = viral cRNA; strain = A/goose/Guangdong/1/1996(H5N1)
tctatcgtcccgtcaggccccctcaaagccgagatcgcgcagagacttgaggatgtctttgcaggaaagaacaccgatctcgaggctctcatggaatggctaaagacaagaccaatcctgtcacctctgactaaagggattttaggatttgtgttcacgctcaccgtgcccagtgagcgaggactgcagcgtagacgctttgtccagaatgccttaaatggaaatggagatccaaacaatatggatagggcagttaagctatacaagaagctgaaaagagaaataacattccatggggctaaggaggtcgcactcagctactcaaccggtgcacttgccagttgtatgggtctcatatacaacaggatgggaacggtgaccacagaagtggcttttggcctagtgtgtgccacttgtgagcagattgcagattcacagcatcggtctcacagacagatggcaactaccaccaacccactaatcaggcatgagaacagaatggtgctggccagcactacagctaaggctatggagcagatggctggatcgagtgagcaggcagcggaagccatggaggttgctagtcaggctaggcagatggtgcaggcaatgaggacaattgggactcatcctagctccagtgccggtctgaaagataatcttcttgaaaatttgcaggcctaccaaaaacgaatgggagtgcaaatgcagcgattcaagtgatcctcttgttgttgccgcaagtatcattgggatactgcacttgatattgtggattcttgatcgtcttttcttcaaatgcatttatcgtcgccttaaatacggtttgaaaagagggccttctacggaaggggtacctgagtctatgagggaagagtatcggcaggaacagcagagtgctgtggatgttgacgatggtcattttgtcaacatagagctggagtaaaaaactaccttgtttctact

SEQ ID NO: 30 - RNA, 865 nt, Influenza A virus
  source 1..865: mol_type = viral cRNA; strain = A/goose/Guangdong/1/1996(H5N1)
gtgacaaagacataatggattccaacacgataacctcgtttcaggtagattgttatctatggcacataagaaagctactcagtatgagagacatgtgtgatgccccctttgatgacaggctccgaagagaccaaaaggcattaaagggaagaggcagcacacttggactcgatttaagagtggctacaatggaggggaaaaagatcgttgaggacatcctgaagagtgagacaaatgaaaacctcaaaatagccattgcttccagtcctgctcctcggtatatcaccgatatgagcatagaggagatgagccgagaatggtacatgctgatgcctaggcagaaaataactggaggccttatggtgaaaatggaccaagccataatggataaaagaattatccttaaagcaaatttctcagttctatttgatcaactagagacattagtctctctgagggcattcacagaaagtggtgctattgtggctgaaatatttcccattccctccgtaccaggacattttacagaggatgtcaaaaatgcaattggaatcctcatcggtggacttgaatggaatgataactcaattcgagcgtctgaaaatatacagagattcgcttggggaatccatgatgagaatgggggaccttcactccctccaaaacagaaacgctacatggcgaaacgagttgagtcagaagtttgaagagatcagatggctcattgctgaatgtagaaatatactgacaaagactgaaaatagctttgaacagataacatttttgcaagcattgcaactcttacttgaagttgagagtgagataaggaccttctcttttcagcttatttaatactaaaaaacac

SEQ ID NO: 31 - RNA, 2368 nt, Influenza B virus
  source 1..2368: mol_type = viral cRNA
agcagaagcggagctttaagatgaatataaatccatattttcttttcatagatgtacctatacaggcagcaatttcaacaacattcccatacaccggtgttcccccttattctcatggaacgggaacaggctacacaatagacaccgtgattagaacacacgagtactcaaacaagggaaaacaatacatttctgatgttacaggatgtgtaatggtagatccaacaaatgggccattacccgaagacaatgaaccgagtgcctatgcacaattggattgtgttctggaggctttggatagaatggatgaagaacatccaggtctgtttcaagcagggtcacagaatgccatggaggcactaatggtcacaacagtggacaaattgactcaggggagacagacctttgattggacggtgtgtagaaaccaacctgctgcaacggcactgaacacaacaataacctcttttaggttgaatgatttaaatggagccgacaagggtggattagtgcccttttgccaagatatcattgattcattagacaaacctgaaatgattttcttcacagtaaagaatataaagaaaaaattgcctgctaaaaacagaaagggtttccttataaaaagaatacctatgaaggtaaaagacagaataacaagagtggaatacatcaaaagagcattatcattaaacacaatgactaaagatgctgaaagaggcaaactaaaaagaagagcaattgccaccgctgggatacaaatcagaggatttgtattagtagttgaaaacttggctaaaaatatctgtgaaaatctagagcaaagtggtttacccgtaggtggaaacgaaaagaaggccaaactatcaaatgcagtggctaaaatgctcagtaattgtccaccaggagggatcagtatgactgtgacaggagacaatactaaatggaatgaatgcttaaatccaagaatctttttggctatgactgaaagaataaccagagacagcccaatttggttccgggatttttgtagtatagcaccggtcttgttctccaataaaatagctagattgggaaaagggttcatgataacaagtaaaacaaaaagactaaaagctcaaataccttgtcccgatctgtttaatataccattagaaagatataatgaagaaacaagggcaaaactgaaaaagctaaaacctttcttcaatgaagaaggaacggcatctctttcgccaggaatgatgatgggaatgtttaatatgctatctacagtattaggagtagccgcactagggataaaaaacattggaaacaaagaatacttatgggatggactgcagtcttcggatgattttgctctgtttgttaatgcaaaagatgaagagacatgtatggaaggaataaacgatttttaccgaacatgtaagctattgggaataaacatgagcaaaaagaaaagttactgtaatgaaactgggatgtttgaatttaccagcatgttttacagagatggatttgtatctaattttgcaatggaactcccttcatttggagtcgctggagtgaatgaatcagcagacatggcaataggaatgacaataataaagaacaatatgatcaacaatgggatgggcccagcaacggcacaaacagccatacaattattcatagctgactatagatacacctacaaatgccacaggggagattccaaagtggaagggaagagaatgaaaattataaaggagctatgggaaaacactaaaggaagagatggtctattagtagcagatggtgggcctaatctttacaatttgagaaacctgcatattccagaaataatattaaaatacaacataatggaccctgagtacaaaggacggttactgcatcctcaaaatccctttgtaggacatttgtctattgagggtatcaaagaagcagatataacacctgcacatggcccaataaagaaaatggactacgatgcggtatctggaactcatagttggagaaccaaaaggaacagatctatactaaacactgatcagaggaacatgattcttgaggaacaatgctacgctaagtgttgcaacctttttgaggcttgctttaacagtgcgtcatacaggaaaccagtaggccagcacagcatgcttgaagctatggcccacagattaagaatggatgcacgactggactatgagtcaggaaggatgtcaaaagaggatttcgaaaaagcaatggctcaccttggtgagattgggtacatgtaagctccggaaatgtctatggggttattggtcatcgttgaatacatgcggtgcacaaatgattaaaatgaaaaaaggctcgtgtttctact

SEQ ID NO: 32 - RNA, 2313 nt, Influenza B virus
  source 1..2313: mol_type = viral cRNA; strain = B/Lee/1940
atgacgttggctaaaattgaactactaaagcagctgttaagggacaatgaagccaaaacggtgttgagacagacaacggtagaccaatacaacataataagaaaattcaatacatcaagaattgaaaagaacccttcattaagaatgaagtgggccatgtgttccaattttcccttagctctgaccaagggtgatatggcaaatcgaatccccttggaatacaagggaatacaacttaaaacaaatgctgaagacataggaactaaaggacaaatgtgttcaatagcagcagttacctggtggaatacatatgggcccataggggatactgaagggtttgaaaaggtctacgaaagcttttttctcagaaagatgagacttgacaatgccacttggggccgaataacctttggccctgttgagagagtaagaaaaagagtactactaaacccgctcaccaaggaaatgcccccagatgaagcgagcaatgtaataatggaaatattattccctaaagaagcaggaataccaagagaatctacttggatacatagagaactgataaaagaaaaaagagaaaaattgaagggaacgatgataactcccattgtactggcatacatgcttgagagagaactagttgcccgaagaaggttcctgccagtagcaggagcaacatcagcagagttcatagaaatgctacattgcttacaaggtgaaaattggagacaaatatatcatccaggagggaataaactaactgaatctagatctcaatcaatgattgtagcttgcaggaagataatcagaagatcaatagttgcatcaaacccactagagctagctgtagagattgcaaataagactgtgatagacactgaacctttaaaatcatgtctggcagccctggatggaggtgatgtagcctgtgacataataagagctgcattaggattaaaaattagacaaagacaaagatttgggagacttgaactaaagagaatatcaggaagaggattcaaaaatgatgaagagatattaatcggaaacggaacaatacaaaagattggaatatgggacggagaagaggaattccatgtaagatgtggcgaatgcagggggatattgaaaaaaagccaaatgagaatggaaaaactactgataaattcagccaaaaaggaggacatgaaagatttaataatcttatgcatggtattttctcaagacactaggatgttccaaggagtgagaggagagataaattttcttaatcgagcaggccaacttttatcccccatgtaccaactccaacgatactttctgaataggagcaatgacctttttgatcaatggggatatgaggaatcacctaaagcaagtgagctacatgggataaatgaattaatgaatgcatctgactatacattgaaaggggttgtagtaacaaaaaatgtgattgatgattttagttctactgaaacagaaaaagtatctataacaaaaaatcttagtttaataaaaaggactggggaagttataatgggagccaatgacgtaagtgaattagaatcacaagcacagctaatgataacgtatgatacacccaagatgtgggaaatgggaacaaccaaagaactggtacaaaacacttaccaatgggtgcttaaaaatttagtaacattgaaggctcagtttcttttgggaaaagaagacatgttccaatgggatgcatttgaagcatttgaaagcataatccctcagaagatggctggtcagtacagtggatttgcaagagcagtgctcaaacaaatgagagaccaagaggttatgaaaactgaccaattcataaaattgttgcctttctgtttttcgccaccaaaattaaggagcaatggagagccttatcaatttttgaggcttatgctgaaaggaggaggggaaaatttcatcgaagtaaggaaagggtcccccttgttctcctacaatccacaaacggaaatcctaactatatgcggcagaatgatgtcattaaaaggaaaaattgaggatgaagaaagaaatagatcaatggggaatgcagtactggcaggctttcttgttagtggcaaatatgaccctgatcttggagatttcaaaaccattgaggaacttgaaagactaaaaccgggagaaaaagccaacatcttactttaccaaggaaagcccgttaaagtagttaaaaggaaaagatatagtgctttatccaatgatatttcacaagggattaagagacaaagaatgacagttgagtccatggggtgggccttgagctaa

SEQ ID NO: 33 - RNA, 2204 nt, Influenza B virus
  source 1..2204: mol_type = viral cRNA; strain = B/Lee/1940
atggatacttttattacaaagaatttccagactacaataatacaaaaggccaaaaacacaatggcagaatttagtgaagatcctgaattacagccagcagtactattcaacatctgcgtccatctggaggtctgctatgtaataagtgatatgaactttcttgatgaggaaggaaagacatatacagcattagaaggacaaggaaaagagcaaaatttgagaccacagtatgaagtgattgagggaatgccaagaaacatagcatggatggttcaaagatccttagcccaagagcatggaatagagactccaaggtatctggctgatttatttgattataaaaccaagaggtttatcgaagtcggaataacaaagggattggctgatgattacttttggaaaaagaaagaaaagttggggaatagcatggaactgatgatattcagctacaatcaagactactcgttaagtgatgaatcttcattggatgaggaaggaaaagggagagtgctaagcagactcacagaacttcaggctgagttaagtttgaaaaacctatggcaagttctaataggggaagaagaaattgaaaaaggaattgacttcaaacttggacaaacaatatctaaactgaggaatatatctgttccagctggtttctccaattttgaagggatgagaagttacatagacaacatagaccctaaaggagcaatagagagaaatctagcaaggatgtctcccttagtatcagttacacccaaaaagttgaaatgggaggacctgagacccatagggcctcacatttacaaccatgagctaccagaagttccatataatgcctttctcctcatgtctgatgagttggggctggccaatatgactgaaggaaagtccaagaaaccgaagaccttagctaaggaatgtctagaaaggtattcaacactacgtgatcaaactgacccaatattgataatgaaaagcgaaaaagctaacgaaaacttcttatggaggttatggagggactgtgtaaatacaataagcaatgaggaaacaggcaacgaattacagaaaaccaattatgccaagtgggccacaggagatggactaacataccaaaaaataatgaaagaagtagcaatagatgacgaaacgatgtaccaagaagaacccaaaatacccaataaatgtagagtggctgcttgggttcaggcagagatgaatctactgagtactctgacaagtaaaagggccctggatctgccagaaatagggccagatgtagcacccgtggagcatgtagggagtgaaagaaggaaatactttgttaatgaaatcaactactgtaaagcctctacagttatgatgaagtatgtactttttcacacttcattattaaatgaaagcaatgctagtatgggaaaatataaagtaataccaatcaccaacagagtggtaaatgaaaaaggggaaagctttgacatgctttatggtctggcggttaaggggcaatctcatttgcggggggacacggatgttgtaacagttgtgactttcgagtttagtagtacagatcctagagtggactcaggaaagtggccaaaatatactgtctttaaaattggctccctatttgtgagtggaagagaaaaacctgtgtacctatattgccgagtgaatggtacaaacaaaatccaaatgaaatggggaatggaagctagaagatgtctgcttcaatcaatgcaacaaatggaggcaattgttgatcaagaatcatcgatacaagggtatgatatgaccaaagcttgtttcaagggagacagagtgaataatcccaaaactttcagtattgggactcaggaaggcaaactagtaaaagggtcctttgggaaagcactaagagtaatattcaccaaatgtttgatgcattatgtatttggaaatgctcaattggaggggtttagtgccgaatctaggagacttctactgttaattcaggcattaaaagacaggaagggcccttgggtatttgacttggagggaatgtactttggagtagaggaatgtattagtaacaatccttgggtaatacagagtgcatactggtttaatgaatggttgggcattgaaaaagaaggaagtaaagtgttagaatcaatagatgaaataatggatgaatgaacgaagggcatagcgctcaattt

SEQ ID NO: 34 - RNA, 1841 nt, Influenza B virus
  source 1..1841: mol_type = viral cRNA; strain = B/Lee/1940
ggcagaagcacagcattttcttgtgagcttcgagcactaataaaactgaaaatcaaaatgtccaacatggatattgacagtataaataccggaacaatcgataaaacaccagaagaactgactcccggaaccagtggggcaaccagaccaatcatcaagccagcaacccttgctccgccaagcaacaaacgaacccgaaatccatctccagaaaggacaaccacaagcagtgaaaccgatatcggaaggaaaatccaaaagaaacaaaccccaacagagataaagaagagcgtctacaaaatggtggtaaaactgggtgaattctacaaccagatgatggtcaaagctggacttaatgatgacatggaaaggaatctaattcaaaatgcacaagctgtggagagaatcctattggctgcaactgatgacaagaaaactgaataccaaaagaaaaggaatgccagagatgtcaaagaagggaaggaagaaatagaccacaacaagacaggaggcaccttttataagatggtaagagatgataaaaccatctacttcagccctataaaaattacctttttaaaagaagaggtgaaaacaatgtacaagaccaccatggggagtgatggtttcagtggactaaatcacattatgattggacattcacagatgaacgatgtctgtttccaaagatcaaagggactgaaaagggttggacttgacccttcattaatcagtacttttgccggaagcacactacccagaagatcaggtacaactggtgttgcaatcaaaggaggtggaactttagtggatgaagccatccgatttataggaagagcaatggcagacagagggctactgagagacatcaaggccaagacggcctatgaaaagattcttctgaatctgaaaaacaagtgctctgcgccgcaacaaaaggctctagttgatcaagtgatcggaagtaggaacccagggattgcagacatagaagacctaactctgcttgccagaagcatggtagttgtcagaccctctgtagcgagcaaagtggtgcttcccataagcatttatgctaaaatacctcaactaggattcaataccgaagaatactctatggttgggtatgaagccatggctctttataatatggcaacacctgtttccatattaagaatgggagatgacgcaaaagataaatctcaactattcttcatgtcgtgcttcggagctgcctatgaagatctaagagtgttatctgcactaacgggcaccgaatttaagcctagatcagcactaaaatgcaagggtttccatgtcccggctaaggagcaagtagaaggaatgggggcagctctgatgtccatcaagcttcagttctgggccccaatgaccagatctggagggaatgaagtaagtggagaaggagggtctggtcaaataagttgcagccctgtgtttgcagtagaaagacctattgctctaagcaagcaagctgtaagaagaatgctgtcaatgaacgttgaaggacgtgatgcagatgtcaaaggaaatctactcaaaatgatgaatgattcaatggcaaagaaaaccagtggaaatgctttcattgggaagaaaatgtttcaaatatcagacaaaaacaaagtcaatcccattgagattccaattaagcagaccatccccaatttcttctttgggagggacacagcagaggattatgatgacctcgattattaaagcaataaaatagacactatggctgtgactgtttcagtacgtttgggatgtgggtgtttactcttattgaaataaatgtaaaaaatgctgttgtttctact

SEQ ID NO: 35 - RNA, 1191 nt, Influenza B virus
  source 1..1191: mol_type = viral cRNA; strain = B/Lee/1940
agcagaagcacgcactttcttaaaatgtcgctgtttggagacacaattgcctacctgctttcactaatagaagatggagaaggcaaagcagaactagctgaaaaattacactgttggttcggtgggaaagaatttgacctagattctgctttggaatggataaaaaacaaaaggtgcctaactgatatacaaaaagcactaattggtgcctctatatgctttttaaaacccaaagaccaagaaagaaaaaggagattcatcacagagcccctgtcaggaatgggaacaacagcaacaaagaagaaaggcctaattctagctgagagaaaaatgagaagatgtgtaagctttcatgaagcatttgaaatagcagaaggccacgaaagctcagcattactatattgtcttatggtcatgtacctaaaccctgaaaactattcaatgcaagtaaaactaggaacgctctgtgctttatgcgagaaacaagcatcgcactcgcatagagcccatagcagagcagcaaggtcttcggtacctggagtaagacgagaaatgcagatggtttcagctatgaacacagcaaagacaatgaatggaatgggaaagggagaagacgtccaaaaactagcagaagagctgcaaaacaacattggagtgttgagatctctaggagcaagtcaaaagaatggagaaggaattgccaaagatgtaatggaagtgctaaaacagagctctatgggaaattcagctcttgtgaggaaatacttataatgctcgaaccacttcagattctttcaatttgttctttcattttatcagctctccatttcatggcttggacaatagggcatttgaatcaaataaaaagaggggtaaacttgaaaatacaaataaggaatccaaataaggaggcaataaacagagaggtgtcaattctgagacacaattaccaaaaggaaatccaagccaaagaaacaatgaagaaaatactctctgacaacatggaagtattgggtgaccacatagtagttgaagggctttcaactgatgagataataaaaatgggtgaaacagttttggaggtggaagaattgcaatgagcccaattttcactgtatttcttactatgcatttaagcaaattgtaatcaatgtcagtgaataaaactggaaaaagtgcgttgtttctact

SEQ ID NO: 36 - RNA, 1096 nt, Influenza B virus
  source 1..1096: mol_type = viral cRNA; strain = B/Lee/1940
cgcagaagcagaggatttatttagtcactggcaaacggaaagatggcggacaacatgaccacaacacaaattgaggtgggtccgggagcaaccaatgccactataaactttgaagcaggaattctggagtgctatgaaaggttttcatggcaaagagcccttgactatcctggtcaagaccgcctacacagactaaaacgaaaattagaatcaagaataaagactcacaacaagagtgagcctgagaataaaaggatgtctcttgaagagagaaaagcaattggggtaaaaatgatgaaagtgcttctgtttatggatccctctgctggaattgaagggtttgagccatactgtgtgaaaaatccctcaactagcaaatgtccaaattacgattggaccgattaccctccaaccccaggaaagtaccttgatgacatagaagaagagccggaaaatgtcgatcacccaattgaggtagtattaagggacatgaacaataaagatgcacgacaaaagataaaggatgaagtaaacactcagaaagaggggaaattccgtttgacaataaaaagggatatacgtaatgtgttgtccttgagagtgttggtgaacggaaccttcctcaagcaccctaatggagacaagtccttatcaactcttcatagattgaatgcatatgaccagaatggagggcttgttgctaaacttgttgctactgatgatcggacagtggaggatgaaaaagatggccatcggatcctcaactcactcttcgagcgttttgatgaaggacattcaaagccaattcgagcagctgaaactgcggtgggagtcttatcccaatttggtcaagagcaccgattatcaccagaagagggagacaattagactggccacggaagaactttatctcttgagtaaaagaattgatgatagtatattgttccacaaaacagtaatagctaacagctccataatagctgacatgattgtatcattatcattactggaaacattgtatgaaatgaaggatgtggttgaagtgtacagcaggcagtgcttatgaatgtaaaataaaaatcctcttgttactact

SEQ ID NO: 37-38 - skipped (2 entries)

SEQ ID NO: 39 - protein, 567 aa, synthetic construct
  source 1..567: note = Chimeric_H6/Turkey/Massachusetts/3740/1965_A151D_Pr otein
MKAKLLVLLYAFVATDADTICIGYHANNSTTQVDTILEKNVTVTHSVELLESQKEERFCRVLNKTPLDLKGCTIEGWILGNPQCDILLGDQSWSYIVERPGAQNGICYPGVLNEVEELKAFIGSGEKVQRFEMFPKSTWTGVDTNSGVTRDCPYTTSGSSFYRNLLWIIKTRSAAYPVIKGTYNNTGSQPILYFWGVHHPPNTDEQNTLYGSGDRYVRMGTESMNFAKSPEIAARPAVNGQRGRIDYYWSVLKPGETLNVESNGNLIAPWYAYKFTSSNNKGAIFKSNLPIENCDAVCQTVAGALKTNKTFQNVSPLWIGECPKYVKSESLRLATGLRNVPQAETRGLFGAIAGFIEGGWTGMIDGWYGYHHENSQGSGYAADKESTQKAIDGITNKVNSIIDKMNTQFEAVEHEFSNLERRIDNLNKRMEDGFLDVWTYNAELLVLLENERTLDLHDANVKNLYEKVKSQLRDNAKDLGNGCFEFWHKCDDECINSVKNGTYDYPKYQDESKLNRQEIDSVKLESMGVYQILAIYSTVASSLVLLVSLGAISFWMCSNGSLQCRIC

SEQ ID NO: 40 - protein, 568 aa, synthetic construct
  source 1..568: note = Chimeric_H6/Turkey/Massachusetts/3740/1965_A151D_V2 _Protein
MKAKLLVLLYAFVATDADTICIGYHANNSTTQVDTILEKNVTVTHSVELLESQKEERFCRVLNKTPLDLKGCTIEGWILGNPQCDILLGDQSWSYIVERPGAQNGICYPGVLNEVEELKAFIGSGEKVQRFEMFPKSTWTGVDTNSGVTRDCPYTTSGSSFYRNLLWIIKTRSAAYPVIKGTYNNTGSQPILYFWGVHHPPNTDEQNTLYGSGDRYVRMGTESMNFAKSPEIAARPAVNGQRGRIDYYWSVLKPGETLNVESNGNLIAPWYAYKFTSSNNKGAIFKSNLPIENCDAVCQTVAGALKTNKTFQNVSPLWIGECPKYVKSESLRLATGLRNVPQAETRGLFGAIAGFIEGGWTGMIDGWYGYHHENSQGSGYAADKESTQKAIDGITNKVNSIIDKMNTQFEAVEHEFSNLERRIDNLNKRMEDGFLDVWTYNAELLVLLENERTLDLHDANVKNLYEKVKSQLRDNAKDLGNGCFEFWHKCDDECINSVKNGTYDYPKYQDESKLNRQEIDSVKLENLGVYQILAIYSTVSSSLVLVGLIIAMGLWMCSNGNIRCNICI

SEQ ID NO: 41 - protein, 568 aa, synthetic construct
  source 1..568: note = Chimeric_H6/Turkey/Massachusetts/3740/1965_R235M_Pr otein
MKAKLLVLLYAFVATDADTICIGYHANNSTTQVDTILEKNVTVTHSVELLESQKEERFCRVLNKTPLDLKGCTIEGWILGNPQCDILLGDQSWSYIVERPGAQNGICYPGVLNEVEELKAFIGSGEKVQRFEMFPKSTWTGVDTNSGVTRACPYTTSGSSFYRNLLWIIKTRSAAYPVIKGTYNNTGSQPILYFWGVHHPPNTDEQNTLYGSGDRYVRMGTESMNFAKSPEIAAMPAVNGQRGRIDYYWSVLKPGETLNVESNGNLIAPWYAYKFTSSNNKGAIFKSNLPIENCDAVCQTVAGALKTNKTFQNVSPLWIGECPKYVKSESLRLATGLRNVPQAETRGLFGAIAGFIEGGWTGMIDGWYGYHHENSQGSGYAADKESTQKAIDGITNKVNSIIDKMNTQFEAVEHEFSNLERRIDNLNKRMEDGFLDVWTYNAELLVLLENERTLDLHDANVKNLYEKVKSQLRDNAKDLGNGCFEFWHKCDDECINSVKNGTYDYPKYQDESKLNRQEIDSVKLENLGVYQILAIYSTVSSSLVLVGLIIAMGLWMCSNGNIRCNICI

SEQ ID NO: 42 - protein, 566 aa, synthetic construct
  source 1..566: note = H8/Mallard/Sweden/24/2002_E408G_Protein
MKAKLLVLLYAFVATDADTICIGYQSNNSTDTVNTLIEQNVPVTQTMELVETEKHPAYCNTDLGAPLELRDCKIEAVIYGNPKCDIHLKDQGWSYIVERPSAPEGMCYPGSVENLEELRFVFSSAASYKRIRLFDYSRWNVTRSGTSKACNASTGGQSFYRSINWLTKKKPDTYDFNEGTYINNEDGDIIFLWGIHHPPDAKEQTTLYKNANTLSSVTTNTINRSFQPNIGPRPLVRGQQGRMDYYWGILKRGETLKIRTNGNLIAPEFGYLLKGESHGRIIQNEDIPIGSCHTKCQTYAGAINSSKPFQNASRHYMGECPKYVKKESLRLAVGLRNTPSIEPRGLFGAIAGFIEGGWSGMIDGWYGFHHSNSEGTGMAADQKSTQEAIDKITNKVNNIVDKMNREFGVVNHEFSEVEKRINMINDKIDDQIEDLWAYNAELLVLLENQKTLDEHDSNVKNLFDEVRRRLSANAIDTGNGCFDILHKCDNECMETIKNGTYNHKEYEEEAKLERSKINGVKLESMGVYQILAIYSTVASSLVLLVSLGAISFWMCSNGSLQCRICI

SEQ ID NO: 43 - RNA, 89 nt, synthetic construct
  source 1..89: mol_type = other RNA; note = Constantregion_U12-signalpeptide_packagingsignal_nu cleotide
agcaaaagcaggggaaaataaaaacaaccaaaatgaaggcaaaactactggtcctgttatatgcatttgtagctacagatgcagacaca

SEQ ID NO: 44 - RNA, 314 nt, synthetic construct
  source 1..314: mol_type = other RNA; note = Constantregion_packagingsignal-U13_v1_nucleotide
atattctacagtggcaagctccttagtactgctagtttctttaggagcgattagcttttggatgtgctccaacggctccctacaatgtcggatttgtatttaatagnnnnnnnnnnnnnnnnagatcggaagagcgtcgtgtagggaaagagtgtgcggccgctatctactcaactgtcgccagttcactggtgctttaggtctccctgggggcaatcagtttctggatgtgttctaatgggtctttgcagtgcagaatatgcatctgagattaggatttcagaaatataaggaaaaacacccttgtttctact

SEQ ID NO: 45 - RNA, 242 nt, synthetic construct
  source 1..242: mol_type = other RNA; note = Constantregion_packagingsignal-U13_v2_nucleotide
ggcaacatccgatgcaacatctgtatataatgagnnnnnnnnnnnnnnnnagatcggaagagcgtcgtgtagggaaagagtgtgcggccgctatctactcaactgtcgccagttcactggtgctttaggtctccctgggggcaatcagtttctggatgtgttctaatgggtctttgcagtgcagaatatgcatctgagattaggatttcagaaatataaggaaaaacacccttgtttctact